(19) United States
(12) Patent Application Publication
(10) Pub. No.: US 2020/0291488 A1
(43) Pub. Date: Sep. 17, 2020

(54) NMR METHODS AND SYSTEMS FOR THE RAPID DETECTION OF CANDIDA SPECIES

(71) Applicant: T2 Biosystems, Inc., Lexington, MA (US)

(72) Inventors: Brendan John MANNING, Arlington, MA (US); Jessica Lee SNYDER, Woburn, MA (US); Benjamin Nguyen CHANG, Cambridge, MA (US); Trissha Ritsue HIGA, Brighton, MA (US); Robert Patrick SHIVERS, Watertown, MA (US); Yin Shan Cathy WONG, Newton, MA (US); Thomas Jay LOWERY, Jr., Belmont, MA (US); Urvi VED, Natick, MA (US); Daniel GAMERO, Medford, MA (US)

(21) Appl. No.: 16/613,702

(22) PCT Filed: May 17, 2018
(86) PCT No.: PCT/US2018/033278

§ 371 Date: Nov. 14, 2019

Related U.S. Application Data
(60) Provisional application No. 62/507,642, filed on May 17, 2017.

(51) Int. Cl. C12Q 1/6895; C12N 1/06; G01N 33/543; C12Q 1/06; C12Q 1/6806; C12N 15/11; A61K 38/08; G01N 24/08; G01R 33/44

(52) U.S. Cl. CPC C12Q 1/6895; C12N 1/063; G01N 33/54326; C12Q 1/06; C12Q 1/6806; G01N 2333/40; A61K 38/08; G01N 24/08; G01R 33/448; G01N 2800/26; C12N 15/11

(57) ABSTRACT

The invention features methods, systems, and panels for rapid detection of Candida species (e.g., Candida auris, Candida lusitaniae, Candida haemulonii, Candida duobushaemulonii, and Candida pseudohaemulonii) in biological samples (e.g., whole blood) and environmental samples (e.g., environmental swabs, e.g., surface swabs), and for diagnosis and monitoring of diseases, including Candidiasis and sepsis.

### SEQUENCE LISTING

The instant application contains a Sequence Listing which has been submitted electronically in ASCII format and is hereby incorporated by reference in its entirety. Said ASCII copy, created on May 17, 2018, is named 50713-118WO2_Sequence_Listing_5.17.18_ST25 and is 12,346 bytes in size.

### FIELD OF THE INVENTION

The invention features methods, panels, and systems for detecting Candida auris and other Candida species and for diagnosing and treating diseases.

### BACKGROUND OF THE INVENTION

Candida auris is now recognized worldwide as a virulent pathogen that is difficult to manage, resulting in high mortality rates. The majority of Candida auris isolates have exhibited resistance to one or more antifungal agents. Nosocomial infections caused by Candida auris are growing due to the increasing rate of colonization and environmental causes. The diagnostic tests available for the identification of Candida auris are limited to date. Additionally, microbiological cultures and subsequent identification of Candida species typically require 2-5 days, and have a sensitivity of approximately 50%. Accurate diagnosis of a Candida auris infection is also hampered by misidentification of C. auris as other species, commonly Candida haemulonii and Saccharomyces cerevisiae.

Thus, there remains a need for rapid and sensitive methods, preferably requiring minimal or no sample preparation, for detecting the presence of Candida auris and other Candida species analytes for diagnosis and monitoring of diseases, including Candidiasis, Candidemia, and sepsis.

### SUMMARY OF THE INVENTION

The invention features methods, panels, and systems for detecting Candida auris and other Candida species (e.g., Candida lusitaniae, Candida haemulonii, Candida duobushaemulonii, and Candida pseudohaemulonii), and diagnosing and treating diseases, including Candidiasis, Candidemia, and sepsis.

In a first aspect, the invention features a method for detecting the presence of a Candida species in a biological or environmental sample, wherein the Candida species is Candida auris, the method including: (a) providing a biological or environmental sample; (b) amplifying a Candida species target nucleic acid in the biological or environmental sample; and (c) detecting the amplified nucleic acid to determine whether Candida auris is present in the biological or environmental sample, wherein (i) the presence of Candida auris in the biological or environmental sample is determined within about 5 hours (e.g., about 1, 2, 3, 4, or 5 hours) from obtaining the sample or less; (ii) the presence of Candida auris is determined directly from the biological or environmental sample without a prior culturing step; and/or (iii) the Candida auris is present in the biological or environmental sample at a concentration of about 10 cells/mL of biological or environmental sample or less (e.g., about 1, 2, 3, 4, 5, 6, 7, 9, or 10 cells/mL). In some embodiments, step (c) further includes detecting the amplified nucleic acid to determine whether Candida lusitaniae is present. In some embodiments, step (c) further includes detecting the amplified nucleic acid to determine whether Candida haemulonii is present. In some embodiments, step (c) further includes detecting the amplified nucleic acid to determine whether Candida duobushaemulonii is present. In some embodiments, step (c) further includes detecting the amplified nucleic acid to determine whether Candida pseudohaemulonii is present. In some embodiments, the method detects a concentration of Candida auris of 10 cells/mL of biological or environmental sample or less (e.g., about 1, 2, 3, 4, 5, 6, 7, 9, or 10 cells/mL). In some embodiments, step (a) further includes lysing Candida cells present in the biological or environmental sample. In some embodiments, the amplified Candida species target nucleic acid is detected by sequencing (e.g., Sanger sequencing or high-throughput sequencing (e.g., ILLUMINA® sequencing), optical, fluorescent, mass, density, magnetic, chromatographic, and/or electrochemical measurement. In some embodiments, the amplified Candida species target nucleic acid is detected by measuring the T₂ relaxation response of the biological or environmental sample or a portion thereof following contacting the biological or environmental sample or the portion thereof with magnetic particles, wherein the magnetic particles have binding moieties on their surfaces, the binding moieties operative to alter the specific aggregation of the magnetic particles in the presence of the amplified Candida species target nucleic acid.

In another aspect, the invention features a method for detecting the presence of a Candida species in a biological or environmental sample, wherein the Candida species is Candida lusitaniae, the method including: (a) providing a biological or environmental sample; (b) amplifying a Candida species target nucleic acid in the biological or environmental sample; and (c) detecting the amplified nucleic acid to determine whether Candida lusitaniae is present in the biological or environmental sample, wherein (i) the presence of Candida lusitaniae in the biological or environmental sample is determined within about 5 hours from obtaining the sample or less (e.g., about 1, 2, 3, 4, or 5 hours); (ii) the presence of Candida lusitaniae is determined directly from the biological or environmental sample without a prior culturing step; and/or (iii) the Candida lusitaniae is present in the biological or environmental sample at a concentration of about 10 cells/mL of biological or environmental sample or less (e.g., about 1, 2, 3, 4, 5, 6, 7, 9, or 10 cells/mL). In some embodiments, step (c) further includes detecting the amplified nucleic acid to determine whether Candida auris is present. In some embodiments, step (c) further includes detecting the amplified nucleic acid to determine whether Candida haemulonii is present. In some embodiments, step (c) further includes detecting the amplified nucleic acid to determine whether Candida duobushaemulonii is present. In some embodiments, step (c) further includes detecting the amplified nucleic acid to determine whether Candida pseudohaemulonii is present. In some embodiments, the method detects a concentration of Candida lusitaniae of 10 cells/mL of biological or environmental sample or less (e.g., about 1, 2, 3, 4, 5, 6, 7, 9, or 10 cells/mL). In some embodiments, step (a) further includes lysing Candida cells present in the biological or environmental sample. In some embodiments, the amplified Candida species target nucleic acid is detected by sequencing (e.g., Sanger sequencing or high-throughput sequencing (e.g., ILLUMINA® sequencing), optical, fluorescent, mass, density, magnetic, chromatographic, and/or electrochemical measurement. In some embodiments, the amplified Candida species target nucleic acid is detected by measuring the T₂ relaxation response of the biological or environmental sample or a portion thereof following contacting the biological or environmental sample or the portion thereof with magnetic particles, wherein the magnetic particles have binding moieties on their surfaces, the binding moieties operative to alter the specific aggregation of the magnetic particles in the presence of the amplified Candida species target nucleic acid.

In another aspect, the invention features a method for detecting the presence of a Candida species in a biological or environmental sample, wherein the Candida species is Candida haemulonii, the method including: (a) providing a biological or environmental sample; (b) amplifying a Candida species target nucleic acid in the biological or environmental sample; and (c) detecting the amplified nucleic acid to determine whether Candida haemulonii is present in the biological or environmental sample, wherein (i) the presence of Candida haemulonii in the biological or environmental sample is determined within about 5 hours (e.g., about 1, 2, 3, 4, or 5 hours) from obtaining the sample or less; (ii) the presence of Candida haemulonii is determined directly from the biological or environmental sample without a prior culturing step; and/or (iii) the Candida haemulonii is present in the biological or environmental sample at a concentration of about 10 cells/mL of biological or environmental sample or less (e.g., about 1, 2, 3, 4, 5, 6, 7, 9, or 10 cells/mL). In some embodiments, step (c) further includes detecting the amplified nucleic acid to determine whether Candida auris is present. In some embodiments, step (c) further includes detecting the amplified nucleic acid to determine whether Candida lusitaniae is present. In some embodiments, step (c) further includes detecting the amplified nucleic acid to determine whether Candida duobushaemulonii is present. In some embodiments, step (c) further includes detecting the amplified nucleic acid to determine whether Candida pseudohaemulonii is present. In some embodiments, the method detects a concentration of Candida haemulonii of 10 cells/mL of biological or environmental sample or less (e.g., 1, 2, 3, 4, 5, 6, 7, 9, or 10 cells/mL). In some embodiments, step (a) further includes lysing Candida cells present in the biological or environmental sample. In some embodiments, the amplified Candida species target nucleic acid is detected by sequencing (e.g., Sanger sequencing or high-throughput sequencing (e.g., ILLUMINA® sequencing), optical, fluorescent, mass, density, magnetic, chromatographic, and/or electrochemical measurement. In some embodiments, the amplified Candida species target nucleic acid is detected by measuring the T₂ relaxation response of the biological or environmental sample or a portion thereof following contacting the biological or environmental sample or the portion thereof with magnetic particles, wherein the magnetic particles have binding moieties on their surfaces, the binding moieties operative to alter the specific aggregation of the magnetic particles in the presence of the amplified Candida species target nucleic acid.

In another aspect, the invention features a method for detecting the presence of a Candida species in a biological or environmental sample, wherein the Candida species is Candida duobushaemulonii, the method including: (a) providing a biological or environmental sample; (b) amplifying a Candida species target nucleic acid in the biological or environmental sample; and (c) detecting the amplified nucleic acid to determine whether Candida duobushaemulonii is present in the biological or environmental sample, wherein (i) the presence of Candida duobushaemulonii in the biological or environmental sample is determined within about 5 hours (e.g., about 1, 2, 3, 4, or 5 hours) from obtaining the sample or less; (ii) the presence of Candida duobushaemulonii is determined directly from the biological or environmental sample without a prior culturing step; and/or (iii) the Candida duobushaemulonii is present in the biological or environmental sample at a concentration of about 10 cells/mL of biological or environmental sample or less (e.g., about 1, 2, 3, 4, 5, 6, 7, 9, or 10 cells/mL). In some embodiments, step (c) further includes detecting the amplified nucleic acid to determine whether Candida auris is present. In some embodiments, step (c) further includes detecting the amplified nucleic acid to determine whether Candida lusitaniae is present. In some embodiments, step (c) further includes detecting the amplified nucleic acid to determine whether Candida haemulonii is present. In some embodiments, step (c) further includes detecting the amplified nucleic acid to determine whether Candida pseudohaemulonii is present. In some embodiments, the method detects a concentration of Candida duobushaemulonii of 10 cells/mL of biological or environmental sample or less (e.g., 1, 2, 3, 4, 5, 6, 7, 9, or 10 cells/mL). In some embodiments, step (a) further includes lysing Candida cells present in the biological or environmental sample. In some embodiments, the amplified Candida species target nucleic acid is detected by sequencing (e.g., Sanger sequencing or high-throughput sequencing (e.g., ILLUMINA® sequencing), optical, fluorescent, mass, density, magnetic, chromatographic, and/or electrochemical measurement. In some embodiments, the amplified Candida species target nucleic acid is detected by measuring the T₂ relaxation response of the biological or environmental sample or a portion thereof following contacting the biological or environmental sample or the portion thereof with magnetic particles, wherein the magnetic particles have binding moieties on their surfaces, the binding moieties operative to alter the specific aggregation of the magnetic particles in the presence of the amplified Candida species target nucleic acid.

In another aspect, the invention features a method for detecting the presence of a Candida species in a biological or environmental sample, wherein the Candida species is Candida pseudohaemulonii, the method including: (a) providing a biological or environmental sample; (b) amplifying a Candida species target nucleic acid in the biological or environmental sample; and (c) detecting the amplified nucleic acid to determine whether Candida pseudohaemulonii is present in the biological or environmental sample, wherein (i) the presence of Candida pseudohaemulonii in the biological or environmental sample is determined within about 5 hours (e.g., about 1, 2, 3, 4, or 5 hours) from obtaining the sample or less; (ii) the presence of Candida pseudohaemulonii is determined directly from the biological or environmental sample without a prior culturing step; and/or (iii) the Candida pseudohaemulonii is present in the biological or environmental sample at a concentration of about 10 cells/mL of biological or environmental sample or less (e.g., about 1, 2, 3, 4, 5, 6, 7, 9, or 10 cells/mL). In some embodiments, step (c) further includes detecting the amplified nucleic acid to determine whether Candida auris is present. In some embodiments, step (c) further includes detecting the amplified nucleic acid to determine whether Candida lusitaniae is present. In some embodiments, step (c) further includes detecting the amplified nucleic acid to determine whether Candida haemulonii is present. In some embodiments, step (c) further includes detecting the amplified nucleic acid to determine whether Candida duobushaemulonii is present. In some embodiments, the method detects a concentration of Candida pseudohaemulonii of 10 cells/mL of biological or environmental sample or less (e.g., 1, 2, 3, 4, 5, 6, 7, 9, or 10 cells/mL). In some embodiments, step (a) further includes lysing Candida cells present in the biological or environmental sample. In some embodiments, the amplified Candida species target nucleic acid is detected by sequencing (e.g., Sanger sequencing or high-throughput sequencing (e.g., ILLUMINA® sequencing), optical, fluorescent, mass, density, magnetic, chromatographic, and/or electrochemical measurement. In some embodiments, the amplified Candida species target nucleic acid is detected by measuring the T₂ relaxation response of the biological or environmental sample or a portion thereof following contacting the biological or environmental sample or the portion thereof with magnetic particles, wherein the magnetic particles have binding moieties on their surfaces, the binding moieties operative to alter the specific aggregation of the magnetic particles in the presence of the amplified Candida species target nucleic acid.

In another aspect, the invention features a method for detecting the presence of Candida species in a biological or environmental sample, wherein the Candida species is Candida auris, the method including: (a) providing a biological or environmental sample; (b) preparing an assay sample by contacting a portion of the biological or environmental sample with magnetic particles, wherein the magnetic particles have binding moieties on their surfaces, the binding moieties operative to alter the specific aggregation of the magnetic particles in the presence of an analyte associated with Candida auris; (c) placing the assay sample in a device, the device including a support defining a well for holding the assay sample, and having an RF coil configured to detect a signal produced by exposing the assay sample to a bias magnetic field created using one or more magnets and an RF pulse sequence; (d) exposing the assay sample to the bias magnetic field and the RF pulse sequence; (e) following step (d), measuring the signal produced by the assay sample; and (f) using the results of step (e) to determine Candida auris is present in the biological or environmental sample. In some embodiments, the method further includes preparing a Candida lusitaniae assay sample by contacting a portion of the biological or environmental sample with magnetic particles, wherein the magnetic particles have binding moieties on their surfaces, the binding moieties operative to alter the specific aggregation of the magnetic particles in the presence of an analyte associated with Candida lusitaniae and determining whether Candida lusitaniae is present in the sample in accordance with steps (c)-(f) of the method. In some embodiments, the method further includes preparing a Candida haemulonii assay sample by contacting a portion of the biological or environmental sample with magnetic particles, wherein the magnetic particles have binding moieties on their surfaces, the binding moieties operative to alter the specific aggregation of the magnetic particles in the presence of an analyte associated with Candida haemulonii and determining whether Candida haemulonii is present in the sample in accordance with steps (c)-(f) of the method. In some embodiments, the method further includes preparing a Candida duobushaemulonii assay sample by contacting a portion of the biological or environmental sample with magnetic particles, wherein the magnetic particles have binding moieties on their surfaces, the binding moieties operative to alter the specific aggregation of the magnetic particles in the presence of an analyte associated with Candida duobushaemulonii and determining whether Candida duobushaemulonii is present in the sample in accordance with steps (c)-(f) of the method. In some embodiments, the method further includes preparing a Candida pseudohaemulonii assay sample by contacting a portion of the biological or environmental sample with magnetic particles, wherein the magnetic particles have binding moieties on their surfaces, the binding moieties operative to alter the specific aggregation of the magnetic particles in the presence of an analyte associated with Candida pseudohaemulonii and determining whether Candida pseudohaemulonii is present in the sample in accordance with steps (c)-(f) of the method.

In another aspect, the invention features a method for detecting the presence of a Candida species cell in a biological or environmental sample, the method including: (a) lysing the Candida species cells in a biological or environmental sample to form a lysate; (b) amplifying a Candida species target nucleic acid in the lysate in the presence of a primer pair to form an amplified lysate including a Candida amplicon, wherein the primer pair includes a forward primer including the oligonucleotide sequence: 5′-GGC ATG CCT GTT TGA GCG T-3′ (SEQ ID NO: 1) or 5′-GGG CAT GCC TGT TTG AGC GT-3′ (SEQ ID NO: 2) and a reverse primer including the oligonucleotide sequence: 5′-GCT TAT TGA TAT GCT TAA GTT CAG CGG GT-3′ (SEQ ID NO: 3); (c) following step (b), contacting the amplified lysate with magnetic particles to form an assay sample, wherein the magnetic particles include binding moieties on their surface, the binding moieties operative to alter aggregation of the magnetic particles in the presence of the Candida species amplicon; (d) providing the assay sample in a detection tube within a device, the device including a support defining a well for holding the detection tube including the assay sample, and having an RF coil configured to detect a signal produced by exposing the assay sample to a bias magnetic field created using one or more magnets and an RF pulse sequence; (e) exposing the assay sample to a bias magnetic field and an RF pulse sequence; (f) following step (e), measuring the signal from the assay sample; and (g) on the basis of the result of step (f), determining whether a Candida species cell was present in the biological or environmental sample. In some embodiments, the magnetic particles include a first population of magnetic particles conjugated to a first probe, and a second population of magnetic particles conjugated to a second probe, the first probe operative to bind to a first segment of the Candida species amplicon and the second probe operative to bind to a second segment of the Candida species amplicon, wherein the magnetic particles form aggregates in the presence of the Candida species amplicon. In some embodiments, the Candida species is Candida auris, and the first probe includes the oligonucleotide sequence: 5′-CTA CCT GAT TTG AGG CGA CAA CAA AAC-3′ (SEQ ID NO: 4), and the second probe includes the oligonucleotide sequence: 5′-CCG CGA AGA TTG GTG AGA AGA CAT-3′ (SEQ ID NO: 5). In some embodiments, the Candida species is Candida lusitaniae, and the first probe includes the oligonucleotide sequence: 5′-CCT ACC TGA TTT GAG GGC GAA ATG TC-3′ (SEQ ID NO: 6), and the second probe includes the oligonucleotide sequence: 5′-GGA GCA ACG CCT AAC CGG G-3′ (SEQ ID NO: 7). In some embodiments, the Candida species is Candida haemulonii, and the first probe includes the oligonucleotide sequence: 5′-GTC CTA CCT GAT TTG AGG GGA AAA AGC-3′ (SEQ ID NO: 8), and the second probe includes the oligonucleotide sequence: 5′-AAC AAA TCC ACC AAC GGT GAG AAG ATA T-3′ (SEQ ID NO: 9). In some embodiments, the Candida species is Candida duobushaemulonii, and the first probe includes the oligonucleotide sequence: 5′-CGT AGA CTT CGC TGC GGA T-3′ (SEQ ID NO: 48) or 5′-GCG TAG ACT TCG CTG CGG AT-3′ (SEQ ID NO: 28), and the second probe includes the oligonucleotide sequence: 5′-CTG GGC GGT GAG AAG AAA TC-3′ (SEQ ID NO: 29). In some embodiments, the Candida species is Candida duobushaemulonii, and the first probe includes the oligonucleotide sequence: 5′-CGT AGA CTT CGC TGC GGA T-3′ (SEQ ID NO: 48) and the second probe includes the oligonucleotide sequence: 5′-CTG GGC GGT GAG AAG AAA TC-3′ (SEQ ID NO: 29). In some embodiments, the Candida species is Candida pseudohaemulonii, and the first probe includes the oligonucleotide sequence: 5′-GCG TAG ACT TCG CTG CTG GAA-3′ (SEQ ID NO: 30), and the second probe includes the oligonucleotide sequence: 5′-CCG TGC GGT GAG AAG AAA TC-3′ (SEQ ID NO: 31). In some embodiments, the Candida species is Candida duobushaemulonii or Candida pseudohaemulonii, and the first probe includes the oligonucleotide sequence: 5′-TCC TAC CTG ATT TGA GGA AAT AGC ATG G-3′ (SEQ ID NO: 32), and the second probe includes the oligonucleotide sequence: 5′-ATT TAG CGG ATG CAA AAC CAC C-3′ (SEQ ID NO: 33).

In some embodiments of any of the preceding aspects, the biological or environmental sample or portion thereof is between about 0.1 and about 4 mL (e.g., about 0.1 mL, about 0.5 mL, about 1 mL, about 1.5 mL, about 1.75 mL, about 2 mL, about 2.5 mL, about 3 mL, about 3.5 mL, or about 4 mL). In some embodiments, the biological or environmental sample is between 1.25 and 2 mL.

In some embodiments of any of the preceding aspects, the biological or environmental sample is blood, a swab, cerebrospinal fluid (CSF), urine, or synovial fluid. In some embodiments, the biological sample is blood. In some embodiments, the blood is whole blood. In some embodiments, amplifying is in the presence of whole blood proteins and non-target nucleic acids. In some embodiments, the biological sample is a swab. In some embodiments, the swab is an environmental swab (e.g., a surface swab) or an epithelial swab (e.g., a buccal swab, an axilla swab, a groin swab, or an axilla/groin swab. In some embodiments, the environmental sample is an environmental swab (e.g., a surface swab). In some embodiments, the swab buffer diluent or swab transport medium is, without limitation, PBST, Amies Buffer, Amies Buffer+10% (v/v) 10×PBST, Cary Blair Media or Liquid Stuart Swabs (which may include addition of 10% (v/v) 10×PBST).

In some embodiments of any of the preceding aspects, lysing includes mechanical lysis or heat lysis. In some embodiments, the mechanical lysis is beadbeating or sonicating.

In some embodiments of any of the preceding aspects, the steps of the method are completed within about 5 hours (e.g., within about 1, 2, 3, 4, or 5 hours). In some embodiments, the steps of the method are completed within 4 hours. In some embodiments, the steps of the method are completed within 3 hours.

In some embodiments of any of the preceding aspects, the assay sample is contacted with 1×10⁶ to 1×10¹³ magnetic particles per milliliter of the biological or environmental sample (e.g., from 1×10⁶ to 1×10⁸, 1×10⁷ to 1×10⁸, 1×10⁷ to 1×10⁹, 1×10⁸ to 1×10¹⁰, 1×10⁹ to 1×10¹¹, or 1×10¹⁰ to 1×10¹³ magnetic particles per milliliter).

In some embodiments of any of the preceding aspects, measuring the signal of the assay sample includes measuring the T₂ relaxation response of the assay sample, and wherein increasing agglomeration in the assay sample produces an increase in the observed T₂ relaxation time of the assay sample.

In some embodiments of any of the preceding aspects, the magnetic particles have a mean diameter of from 150 nm to 699 nm (e.g., from 150 to 250, 200 to 350, 250 to 450, 300 to 500, 450 to 650, or from 500 to 699 nm) or from 700 nm to 1200 nm (e.g., from 700 to 850, 800 to 950, 900 to 1050, or from 1000 to 1200 nm). In some embodiments, the magnetic particles have a mean diameter of from 700 nm to 950 nm (e.g., from 700 to 750, 700 to 800, 700 to 850, or from 700 to 900 nm). In some embodiments, the magnetic particles have a mean diameter of from 700 nm to 850 nm (e.g., about 700 nm, about 725 nm, about 750 nm, about 775 nm, about 800 nm, about 825 nm, or about 850 nm). In some embodiments, the magnetic particles have a T₂ relaxivity per particle of from 1×10⁹ to 1×10¹² mM⁻¹s⁻¹ (e.g., from 1×10⁹ to 1×10¹⁰, 1×10⁹ to 1×10¹¹, or from 1×10¹⁰ to 1×10¹² mM⁻¹s⁻¹).

In some embodiments of any of the preceding aspects, the magnetic particles are substantially monodisperse.

In another aspect, the invention features a method for detecting the presence of a Candida species in a whole blood sample, wherein the Candida species is Candida auris, the method including: (a) providing an extract produced by lysing the red blood cells in a whole blood sample from a subject, centrifuging the sample to form a supernatant and a pellet, discarding some or all of the supernatant, and resuspending the pellet to form an extract, optionally washing the pellet prior to resuspending the pellet and optionally repeating the centrifuging, discarding, and resuspending steps; (b) lysing cells in the extract to form a lysate; (c) amplifying a Candida species target nucleic acid in the lysate to form an amplified lysate solution; (d) following step (c), adding to the amplified lysate solution from 1×10⁶ to 1×10¹³ magnetic particles per milliliter of the amplified lysate solution to form a mixture, wherein the magnetic particles have a mean diameter of from 700 nm to 950 nm and binding moieties on their surface, the binding moieties operative to alter aggregation of the magnetic particles in the presence of a target nucleic acid, wherein said magnetic particles have a T₂ relaxivity per particle of from 1×10⁹ to 1×10¹² mM⁻¹s⁻¹; (e) providing the mixture in a detection tube within a device, the device including a support defining a well for holding the detection tube including the magnetic particles and the target nucleic acid, and having an RF coil disposed about the well, the RF coil configured to detect a signal produced by exposing the mixture to a bias magnetic field created using one or more magnets and an RF pulse sequence; (f) exposing the mixture to a bias magnetic field and an RF pulse sequence; (g) following step (f), measuring the signal from the detection tube; (h) on the basis of the result of step (g), detecting the target nucleic acid, wherein step (g) is carried out without any prior purification of the amplified lysate solution; and (i) on the basis of the result of step (h), determining whether the Candida species was present in the sample. In some embodiments, the whole blood sample is from 0.05 to 4.0 mL. In some embodiments, step (g) includes measuring the T₂ relaxation response of the mixture, and wherein increasing agglomeration in the mixture produces an increase in the observed T₂ relaxation time of the mixture. In some embodiments, the amplifying of step (c) includes amplifying a nucleic acid to be detected in the presence of a forward primer and a reverse primer, each of which is universal to multiple Candida species to form a solution including a Candida amplicon; and said magnetic particles of step (d) have a first probe and a second probe conjugated to their surface, the first probe operative to bind to a first segment of the target nucleic acid and the second probe operative to bind to a second segment of the target nucleic acid, wherein the magnetic particles form aggregates in the presence of the target nucleic acid. In some embodiments, the forward primer includes the oligonucleotide sequence: 5′-GGC ATG CCT GTT TGA GCG T-3′ (SEQ ID NO: 1) or 5′-GGG CAT GCC TGT TTG AGC GT-3′ (SEQ ID NO: 2). In some embodiments, the forward primer includes the oligonucleotide sequence: 5′-GGC ATG CCT GTT TGA GCG T-3′ (SEQ ID NO: 1). In some embodiments, the reverse primer includes the oligonucleotide sequence: 5′-GCT TAT TGA TAT GCT TAA GTT CAG CGG GT-3′ (SEQ ID NO: 3). In some embodiments, the first probe includes the oligonucleotide sequence: 5′-CTA CCT GAT TTG AGG CGA CAA CAA AAC-3′ (SEQ ID NO: 4), and the second probe includes the oligonucleotide sequence: 5′-CCG CGA AGA TTG GTG AGA AGA CAT-3′ (SEQ ID NO: 5). In some embodiments, the magnetic particles include two populations, a first population bearing the first probe on its surface, and the second population bearing the second probe on its surface. In some embodiments, the method further includes determining whether Candida lusitaniae is present in the sample. In some embodiments, the method further includes determining whether Candida haemulonii is present in the sample. In some embodiments, the method further includes determining whether Candida duobushaemulonii is present in the sample. In some embodiments, the method further includes determining whether Candida pseudohaemulonii is present in the sample.

In some embodiments of any of the preceding aspects, the method detects a concentration of Candida species of about 10 cells/mL of biological or environmental sample or less (e.g., about 1, 2, 3, 4, 5, 6, 7, 9, or 10 cells/mL).

In another aspect, the invention features a composition including: (a) a liquid sample, wherein the liquid sample (i) is suspected of containing at least one Candida auris target nucleic acid, or (ii) contains at least one Candida auris target nucleic acid amplicon generated from an amplification reaction; and (b) within the liquid sample, from 1×10⁶ to 1×10¹³ magnetic particles per milliliter of the liquid sample, the magnetic particles having a mean diameter of from 700 nm to 950 nm, a T₂ relaxivity per particle of from 1×10⁴ to 1×10¹² mM⁻¹s⁻¹, the magnetic particles including a first population and a second population, the first population having a first nucleic acid probe conjugated to their surface and the second population having a second nucleic acid probe conjugated to their surface, wherein the first probe includes the oligonucleotide sequence: 5′-CTA CCT GAT TTG AGG CGA CAA CAA AAC-3′ (SEQ ID NO: 4), and the second probe includes the oligonucleotide sequence: 5′-CCG CGA AGA TTG GTG AGA AGA CAT-3′ (SEQ ID NO: 5).

In another aspect, the invention features a composition including: (a) a liquid sample, wherein the liquid sample (i) is suspected of containing at least one Candida lusitaniae target nucleic acid, or (ii) contains at least one Candida lusitaniae target nucleic acid amplicon generated from an amplification reaction; and

(b) within the liquid sample, from 1×10⁶ to 1×10¹³ magnetic particles per milliliter of the liquid sample (e.g., from 1×10⁶ to 1×10⁸, 1×10⁷ to 1×10⁸, 1×10⁷ to 1×10⁹, 1×10⁸ to 1×10¹⁰, 1×10⁹ to 1×10¹¹, or 1×10¹⁰ to 1×10¹³ magnetic particles per milliliter), the magnetic particles having a mean diameter of from 700 nm to 950 nm (e.g., from 700 to 750, 700 to 800, 700 to 850, or from 700 to 900 nm), a T₂ relaxivity per particle of from 1×10⁴ to 1×10¹² mM⁻¹s⁻¹ (e.g., from 1×10⁹ to 1×10¹² mM⁻¹s⁻¹ (e.g., from 1×10⁹ to 1×10¹⁰, 1×10⁹ to 1×10¹¹, or from 1×10¹⁰ to 1×10¹² mM⁻¹s⁻¹)), the magnetic particles including a first population and a second population, the first population having a first nucleic acid probe conjugated to their surface and the second population having a second nucleic acid probe conjugated to their surface, wherein the first probe includes the oligonucleotide sequence: 5′-CCT ACC TGA TTT GAG GGC GAA ATG TC-3′ (SEQ ID NO: 6), and the second probe includes the oligonucleotide sequence: 5′-GGA GCA ACG CCT AAC CGG G-3′ (SEQ ID NO: 7).

In another aspect, the invention features a composition including: (a) a liquid sample, wherein the liquid sample (i) is suspected of containing at least one Candida haemulonii target nucleic acid, or (ii) contains at least one Candida haemulonii target nucleic acid amplicon generated from an amplification reaction; and (b) within the liquid sample, from 1×10⁶ to 1×10¹³ magnetic particles per milliliter of the liquid sample (e.g., from 1×10⁶ to 1×10⁸, 1×10⁷ to 1×10⁸, 1×10⁷ to 1×10⁹, 1×10⁸ to 1×10¹⁰, 1×10⁹ to 1×10¹¹, or 1×10¹⁰ to 1×10¹³ magnetic particles per milliliter), the magnetic particles having a mean diameter of from 700 nm to 950 nm (e.g., from 700 to 750, 700 to 800, 700 to 850, or from 700 to 900 nm), a T₂ relaxivity per particle of from 1×10⁴ to 1×10¹² mM⁻¹s⁻¹ (e.g., from 1×10⁹ to 1×10¹² mM⁻¹s⁻¹ (e.g., from 1×10⁹ to 1×10¹⁰, 1×10⁹ to 1×10¹¹, or from 1×10¹⁰ to 1×10¹² mM⁻¹s⁻¹)), the magnetic particles including a first population and a second population, the first population having a first nucleic acid probe conjugated to their surface and the second population having a second nucleic acid probe conjugated to their surface, wherein the first probe includes the oligonucleotide sequence: 5′-GTC CTA CCT GAT TTG AGG GGA AAA AGC-3′ (SEQ ID NO: 8), and the second probe includes the oligonucleotide sequence: 5′-AAC AAA TCC ACC AAC GGT GAG AAG ATA T-3′ (SEQ ID NO: 9).

In another aspect, the invention features a composition including: (a) a liquid sample, wherein the liquid sample (i) is suspected of containing at least one Candida duobushaemulonii target nucleic acid, or (ii) contains at least one Candida duobushaemulonii target nucleic acid amplicon generated from an amplification reaction; and (b) within the liquid sample, from 1×10⁶ to 1×10¹³ magnetic particles per milliliter of the liquid sample (e.g., from 1×10⁶ to 1×10⁸, 1×10⁷ to 1×10⁸, 1×10⁷ to 1×10⁹, 1×10⁸ to 1×10¹⁰, 1×10⁹ to 1×10¹¹, or 1×10¹⁰ to 1×10¹³ magnetic particles per milliliter), the magnetic particles having a mean diameter of from 700 nm to 950 nm (e.g., from 700 to 750, 700 to 800, 700 to 850, or from 700 to 900 nm), a T₂ relaxivity per particle of from 1×10⁴ to 1×10¹² mM⁻¹s⁻¹ (e.g., from 1×10⁹ to 1×10¹² mM⁻¹s⁻¹ (e.g., from 1×10⁹ to 1×10¹⁰, 1×10⁹ to 1×10¹¹, or from 1×10¹⁰ to 1×10¹² mM⁻¹s⁻¹)), the magnetic particles including a first population and a second population, the first population having a first nucleic acid probe conjugated to their surface and the second population having a second nucleic acid probe conjugated to their surface, wherein the first probe includes the oligonucleotide sequence: 5′-CGT AGA CTT CGC TGC GGA T-3′ (SEQ ID NO: 48) or 5′-GCG TAG ACT TCG CTG CGG AT-3′ (SEQ ID NO: 28), and the second probe includes the oligonucleotide sequence: 5′-CTG GGC GGT GAG AAG AAA TC-3′ (SEQ ID NO: 29). In some embodiments, the first probe includes the oligonucleotide sequence: 5′-CGT AGA CTT CGC TGC GGA T-3′ (SEQ ID NO: 48) and the second probe includes the nucleotide sequence 5′-CTG GGC GGT GAG AAG AAA TC-3′ (SEQ ID NO: 29).

In another aspect, the invention features a composition including: (a) a liquid sample, wherein the liquid sample (i) is suspected of containing at least one Candida pseudohaemulonii target nucleic acid, or (ii) contains at least one Candida pseudohaemulonii target nucleic acid amplicon generated from an amplification reaction; and (b) within the liquid sample, from 1×10⁶ to 1×10¹³ magnetic particles per milliliter of the liquid sample (e.g., from 1×10⁶ to 1×10⁸, 1×10⁷ to 1×10⁸, 1×10⁷ to 1×10⁹, 1×10⁸ to 1×10¹⁰, 1×10⁹ to 1×10¹¹, or 1×10¹⁰ to 1×10¹³ magnetic particles per milliliter), the magnetic particles having a mean diameter of from 700 nm to 950 nm (e.g., from 700 to 750, 700 to 800, 700 to 850, or from 700 to 900 nm), a T₂ relaxivity per particle of from 1×10⁴ to 1×10¹² mM⁻¹s⁻¹ (e.g., from 1×10⁹ to 1×10¹² mM⁻¹s⁻¹ (e.g., from 1×10⁹ to 1×10¹⁰, 1×10⁹ to 1×10¹¹, or from 1×10¹⁰ to 1×10¹² mM⁻¹s⁻¹)), the magnetic particles including a first population and a second population, the first population having a first nucleic acid probe conjugated to their surface and the second population having a second nucleic acid probe conjugated to their surface, wherein the first probe includes the oligonucleotide sequence: 5′-GCG TAG ACT TCG CTG CTG GAA-3′ (SEQ ID NO: 30), and the second probe includes the oligonucleotide sequence: 5′-CCG TGC GGT GAG AAG AAA TC-3′ (SEQ ID NO: 31).

In another aspect, the invention features a composition including: (a) a liquid sample, wherein the liquid sample (i) is suspected of containing at least one Candida duobushaemulonii or Candida pseudohaemulonii target nucleic acid, or (ii) contains at least one Candida duobushaemulonii or Candida pseudohaemulonii target nucleic acid amplicon generated from an amplification reaction; and (b) within the liquid sample, from 1×10⁶ to 1×10¹³ magnetic particles per milliliter of the liquid sample (e.g., from 1×10⁶ to 1×10⁸, 1×10⁷ to 1×10⁸, 1×10⁷ to 1×10⁹, 1×10⁸ to 1×10¹⁰, 1×10⁹ to 1×10¹¹, or 1×10¹⁰ to 1×10¹³ magnetic particles per milliliter), the magnetic particles having a mean diameter of from 700 nm to 950 nm (e.g., from 700 to 750, 700 to 800, 700 to 850, or from 700 to 900 nm), a T₂ relaxivity per particle of from 1×10⁴ to 1×10¹² mM⁻¹s⁻¹ (e.g., from 1×10⁹ to 1×10¹² mM⁻¹s⁻¹ (e.g., from 1×10⁹ to 1×10¹⁰, 1×10⁹ to 1×10¹¹, or from 1×10¹⁰ to 1×10¹² mM⁻¹s⁻¹)), the magnetic particles including a first population and a second population, the first population having a first nucleic acid probe conjugated to their surface and the second population having a second nucleic acid probe conjugated to their surface, wherein the first probe includes the oligonucleotide sequence: 5′-TCC TAC CTG ATT TGA GGA AAT AGC ATG G-3′ (SEQ ID NO: 32), and the second probe includes the oligonucleotide sequence: 5′-ATT TAG CGG ATG CAA AAC CAC C-3′ (SEQ ID NO: 33).

In another aspect, the invention features a removable cartridge including a plurality of wells, wherein the removable cartridge includes a first well including any of the preceding compositions. In some embodiments, the removable cartridge includes (a′) a first well including features a composition including: (a) a liquid sample, wherein the liquid sample (i) is suspected of containing at least one Candida haemulonii target nucleic acid, or (ii) contains at least one Candida haemulonii target nucleic acid amplicon generated from an amplification reaction; and (b) within the liquid sample, from 1×10⁶ to 1×10¹³ magnetic particles per milliliter of the liquid sample, the magnetic particles having a mean diameter of from 700 nm to 950 nm, a T₂ relaxivity per particle of from 1×10⁴ to 1×10¹² mM⁻¹s⁻¹, the magnetic particles including a first population and a second population, the first population having a first nucleic acid probe conjugated to their surface and the second population having a second nucleic acid probe conjugated to their surface, wherein the first probe includes the oligonucleotide sequence: 5′-GTC CTA CCT GAT TTG AGG GGA AAA AGC-3′ (SEQ ID NO: 8), and the second probe includes the oligonucleotide sequence: 5′-AAC AAA TCC ACC AAC GGT GAG AAG ATA T-3′ (SEQ ID NO: 9); (b′) a second well including a composition including: (a) a liquid sample, wherein the liquid sample (i) is suspected of containing at least one Candida lusitaniae target nucleic acid, or (ii) contains at least one Candida lusitaniae target nucleic acid amplicon generated from an amplification reaction; and (b) within the liquid sample, from 1×10⁶ to 1×10¹³ magnetic particles per milliliter of the liquid sample, the magnetic particles having a mean diameter of from 700 nm to 950 nm, a T₂ relaxivity per particle of from 1×10⁴ to 1×10¹² mM⁻¹s⁻¹, the magnetic particles including a first population and a second population, the first population having a first nucleic acid probe conjugated to their surface and the second population having a second nucleic acid probe conjugated to their surface, wherein the first probe includes the oligonucleotide sequence: 5′-CCT ACC TGA TTT GAG GGC GAA ATG TC-3′ (SEQ ID NO: 6), and the second probe includes the oligonucleotide sequence: 5′-GGA GCA ACG CCT AAC CGG G-3′ (SEQ ID NO: 7); and/or (c′) a third well including a composition including: (a) a liquid sample, wherein the liquid sample (i) is suspected of containing at least one Candida haemulonii target nucleic acid, or (ii) contains at least one Candida haemulonii target nucleic acid amplicon generated from an amplification reaction; and (b) within the liquid sample, from 1×10⁶ to 1×10¹³ magnetic particles per milliliter of the liquid sample, the magnetic particles having a mean diameter of from 700 nm to 950 nm, a T₂ relaxivity per particle of from 1×10⁴ to 1×10¹² mM⁻¹s⁻¹, the magnetic particles including a first population and a second population, the first population having a first nucleic acid probe conjugated to their surface and the second population having a second nucleic acid probe conjugated to their surface, wherein the first probe includes the oligonucleotide sequence: 5′-GTC CTA CCT GAT TTG AGG GGA AAA AGC-3′ (SEQ ID NO: 8), and the second probe includes the oligonucleotide sequence: 5′-AAC AAA TCC ACC AAC GGT GAG AAG ATA T-3′ (SEQ ID NO: 9). In some embodiments, the removable cartridge includes (a′) through (c′).

In some embodiments of the preceding aspect, the removable cartridge further includes: (d′) a fourth well including a composition including: (a) a liquid sample, wherein the liquid sample (i) is suspected of containing at least one Candida duobushaemulonii target nucleic acid, or (ii) contains at least one Candida duobushaemulonii target nucleic acid amplicon generated from an amplification reaction; and (b) within the liquid sample, from 1×10⁶ to 1×10¹³ magnetic particles per milliliter of the liquid sample, the magnetic particles having a mean diameter of from 700 nm to 950 nm, a T₂ relaxivity per particle of from 1×10⁴ to 1×10¹² mM⁻¹s⁻¹, the magnetic particles including a first population and a second population, the first population having a first nucleic acid probe conjugated to their surface and the second population having a second nucleic acid probe conjugated to their surface, wherein the first probe includes the oligonucleotide sequence: 5′-CGT AGA CTT CGC TGC GGA T-3′ (SEQ ID NO: 48) or 5′-GCG TAG ACT TCG CTG CGG AT-3′ (SEQ ID NO: 28), and the second probe includes the oligonucleotide sequence: 5′-CTG GGC GGT GAG AAG AAA TC-3′ (SEQ ID NO: 29); (e′) a fifth well including a composition including: (a) a liquid sample, wherein the liquid sample (i) is suspected of containing at least one Candida pseudohaemulonii target nucleic acid, or (ii) contains at least one Candida pseudohaemulonii target nucleic acid amplicon generated from an amplification reaction; and (b) within the liquid sample, from 1×10⁶ to 1×10¹³ magnetic particles per milliliter of the liquid sample, the magnetic particles having a mean diameter of from 700 nm to 950 nm, a T₂ relaxivity per particle of from 1×10⁴ to 1×10¹² mM⁻¹s⁻¹, the magnetic particles including a first population and a second population, the first population having a first nucleic acid probe conjugated to their surface and the second population having a second nucleic acid probe conjugated to their surface, wherein the first probe includes the oligonucleotide sequence: 5′-GCG TAG ACT TCG CTG CTG GAA-3′ (SEQ ID NO: 30), and the second probe includes the oligonucleotide sequence: 5′-CCG TGC GGT GAG AAG AAA TC-3′ (SEQ ID NO: 31); and/or (f′) a sixth well including a composition including: (a) a liquid sample, wherein the liquid sample (i) is suspected of containing at least one Candida duobushaemulonii or Candida pseudohaemulonii target nucleic acid, or (ii) contains at least one Candida duobushaemulonii or Candida pseudohaemulonii target nucleic acid amplicon generated from an amplification reaction; and (b) within the liquid sample, from 1×10⁶ to 1×10¹³ magnetic particles per milliliter of the liquid sample, the magnetic particles having a mean diameter of from 700 nm to 950 nm, a T₂ relaxivity per particle of from 1×10⁴ to 1×10¹² mM⁻¹s⁻¹, the magnetic particles including a first population and a second population, the first population having a first nucleic acid probe conjugated to their surface and the second population having a second nucleic acid probe conjugated to their surface, wherein the first probe includes the oligonucleotide sequence: 5′-TCC TAC CTG ATT TGA GGA AAT AGC ATG G-3′ (SEQ ID NO: 32), and the second probe includes the oligonucleotide sequence: 5′-ATT TAG CGG ATG CAA AAC CAC C-3′ (SEQ ID NO: 33).

In some embodiments of any of the preceding aspects, the removable cartridge further includes one or more chambers for holding a plurality of reagent modules for holding one or more assay reagents.

In some embodiments, the removable cartridge further includes a chamber including beads for lysing cells. In some embodiments, the removable cartridge further includes chamber including a polymerase. In some embodiments, the removable cartridge further includes chamber including one or more primers. In some embodiments, the one or more primers include oligonucleotide sequences selected from SEQ ID NOs: 1, 2, and 3. In some embodiments, the one or more primers include SEQ ID NO: 1 and SEQ ID NO: 3.

In another aspect, the invention features a method for diagnosing a disease in a subject, the method including: (a) providing a biological sample obtained from the subject; and (b) detecting the presence of a Candida species in the biological sample according to any of the preceding methods or any of the methods described herein, wherein the presence of a Candida species in the biological sample obtained from the subject identifies the subject as one who may have the disease. In some embodiments, the Candida species is Candida auris. In some embodiments, the Candida species is Candida lusitaniae. In some embodiments, the Candida species is Candida haemulonii. In some embodiments, the Candida species is Candida duobushaemulonii. In some embodiments, the Candida species is Candida pseudohaemulonii.

In another aspect, the invention features a method for treating a disease in a subject, the method including administering an effective amount of a therapeutic agent to the subject, wherein the subject has been diagnosed as having the disease based on detecting the presence of a Candida species according to any of the preceding methods or any of the methods described herein. In some embodiments, the Candida species is Candida auris, Candida lusitaniae, Candida haemulonii, Candida duobushaemulonii, or Candida pseudohaemulonii.

In some embodiments of any of the preceding aspects, the disease is Candidiasis, Candidemia, or sepsis. In some embodiments, the therapeutic agent is an antifungal agent. In some embodiments, the subject has a Candida auris infection and the antifungal agent is a 1,3-β-D-glucan synthesis inhibitor. In some embodiments, the 1,3-β-D-glucan synthesis inhibitor is caspofungin, anidulafungin, micafungin, enfumafungin, or SCY-078.

In another aspect, the invention features a method for infection control, decolonization, or epidemiological monitoring, the method comprising: (a) providing an environmental sample; and (b) detecting the presence of a Candida species in the environmental sample according to any of the preceding methods or any of the methods described herein, wherein the presence of a Candida species in the environmental sample is used for infection control, decolonization, or epidemiological monitoring. In some embodiments, the Candida species is Candida auris, Candida lusitaniae, Candida haemulonii, Candida duobushaemulonii, or Candida pseudohaemulonii.

### BRIEF DESCRIPTION OF THE DRAWINGS

FIG. 1 is a graph showing the results of detection of Candida auris spiked at the indicated concentrations into whole blood. The graph shows T2 signal (ms) obtained from the assay described in Example 1 using magnetic particles having probes for the indicated Candida species. C. auris was detected in samples spiked at 10.7 colony-forming units (CFU)/mL and at 5.8 CFU/mL, indicating that the assay described in Example 1 has a limit of detection (LoD) of less than or equal to 10 CFU/mL.

FIG. 2 is a table showing average T2 signal (ms) using the indicated magnetic particle mixes for detection of the indicated species. The bolded numbers indicate positive detection. A/T, particle conjugated with probes specific for Candida albicans and Candida tropicalis. K/G, particle conjugated with probes specific for Candida krusei and Candida glabrata. P, particle conjugated with probes specific for C. parapsilosis. Primer 1 corresponds to SEQ ID NO: 1. IC, internal control.

FIGS. 3A-3D are a series of graphs showing T2 magnetic resonance (T2MR) detection at 40-46 cycles of the T2Cauris PCR reaction at 58° C. annealing temperature for C. auris (FIG. 3A), Candida duobushaemulonii (FIG. 3B), Candida haemulonii (FIG. 3C), and orange internal control (FIG. 3D). The following abbreviations are used throughout: C. au, Candida auris; C. db, Candida duobushaemulonii; C. h, Candida haemulonii; OIC, orange internal control.

FIGS. 3E-3H are a series of graphs showing T2MR detection at 40-46 cycles of the T2Cauris PCR reaction at 61° C. annealing temperature for C. auris (FIG. 3E), Candida duobushaemulonii (FIG. 3F), Candida haemulonii (FIG. 3G), and OIC (FIG. 3H).

FIGS. 4A-4D are a series of graphs showing average T2 signals in buffer for the indicated species and OIC in presence of aluminum chloride (AlCl₃) (FIG. 4A), chlorhexidine (CHX) (FIG. 4B), micafungin (FIG. 4C), and EDTA (FIG. 4D).

FIG. 4E-4H are a series of graphs showing average T2 signals in blood for the indicated species and OIC in presence of AlCl₃ (FIG. 4E), CHX (FIG. 4F), micafungin (FIG. 4G), and EDTA (FIG. 4H).

FIGS. 5A-5D are a series of graphs showing significant factors affecting the T2 signals for all the channels of the T2Cauris panel.

FIGS. 6A-6D are a series of graphs showing analysis of the variance (ANOVA) plots to compare the T2 signals of all the channels in the T2Cauris panel with the variation in the MgCl₂ concentration reduced to ±5%.

FIGS. 7A-7D are a series of graphs showing significant factors affecting the T2 signals for all the channels of the T2Cauris panel.

FIG. 8 is a graph showing the T2 signals of all the channels in the T2Cauris panel with the variation in the pH reduced to +0.125 units and the concentration of dNTPs reduced to +5%.

FIG. 9 is a graph showing the T2 signals of all the channels in the T2Cauris panel to evaluate the equivalency of the triple spikes in PBST and Amies buffers.

FIG. 10 is a graph showing the T2 signals of all the channels in the T2Cauris panel to evaluate the equivalency of the triple spikes in PBST and Amies buffers with the cell bullet spiking method.

FIG. 11 is a graph showing detection of Candida target oligomers.

### DETAILED DESCRIPTION OF EMBODIMENTS OF THE INVENTION

The invention features methods, systems, cartridges, and panels for detection of Candida species (including Candida auris, Candida lusitaniae, Candida haemulonii, Candida duobushaemulonii, and Candida pseudohaemulonii), for example, for detection in biological or environmental samples. The present invention is based, at least in part, on the development of approaches for rapid (e.g., less than 5 hours), sensitive, and specific detection of Candida auris, Candida lusitaniae, Candida haemulonii, Candida duobushaemulonii, and Candida pseudohaemulonii directly in whole blood or other biological or environmental samples, with limits of detection of ≤10 cells/mL.

In some embodiments, the methods and systems of the invention employ magnetic particles. In some embodiments, the methods and systems employ an NMR unit, optionally one or more magnetic assisted agglomeration (MAA) units, optionally one or more incubation stations at different temperatures, optionally one or more vortexers, optionally one or more centrifuges, optionally a fluidic manipulation station, optionally a robotic system, and optionally one or more modular cartridges, as described in International Patent Application Publication No. WO 2012/054639, which is incorporated herein by reference in its entirety. In other instances, the methods and systems of the invention may involve sequencing (e.g., Sanger sequencing or high-throughput sequencing (e.g., ILLUMINA® sequencing), The methods, compositions, systems, and devices of the invention can be used to assay a biological sample (e.g., whole blood, serum, plasma (e.g., platelet-rich plasma or platelet-poor plasma), cerebrospinal fluid (CSF), urine, synovial fluid, breast milk, sweat, tears, saliva, semen, feces, vaginal fluid or tissue (e.g., from a vaginal swab), sputum, nasopharyngeal aspirate or swab, lacrimal fluid, mucous, epithelial swab (e.g., a buccal swab, an axilla swab, a groin swab, or an axilla/groin swab), an environmental sample (e.g., an environmental swab, e.g., a surface swab), tissues (e.g., tissue homogenates), organs, bones, teeth, among others), or culture media (e.g., brain heart infusion (BHI), Sabouraud BHI (SABHI), Sabouraud Dextrose Agar (SDA), Luria Broth (LB), and the like). In some embodiments, the swab buffer diluent or swab transport medium is, without limitation, PBST, Amies Buffer, Amies Buffer+10% (v/v) 10×PBST, Cary Blair Media, or Liquid Stuart Swabs (which may include addition of 10% (v/v) 10×PBST). The methods and systems can be fully automated, for example, using a T2DX® Instrument (T2 Biosystems, Inc.), a fully automated, clinical multiplex, bench top diagnostics system.

### Definitions

The terms “aggregation,” “agglomeration,” and “clustering” are used interchangeably in the context of the magnetic particles described herein and mean the binding of two or more magnetic particles to one another, for example, via a multivalent analyte, multimeric form of analyte, antibody, nucleic acid molecule, or other binding molecule or entity. In some instances, magnetic particle agglomeration is reversible.

As used herein, by “administering” is meant a method of giving a dosage of a composition described herein (e.g., a composition comprising an antifungal agent) to a subject. The compositions utilized in the methods described herein can be administered by any suitable route, e.g., parenteral (for example, intravenous or intraperitoneal), dermal, transdermal, ocular, inhalation, buccal, sublingual, perilingual, nasal, rectal, topical, and oral. The compositions utilized in the methods described herein can also be administered locally or systemically. The preferred method of administration can vary depending on various factors (e.g., the components of the composition being administered and the severity of the condition being treated).

The terms “amplification” or “amplify” or derivatives thereof as used herein mean one or more methods known in the art for copying a target or template nucleic acid, thereby increasing the number of copies of a selected nucleic acid sequence. Amplification may be exponential or linear. A target or template nucleic acid may be either DNA or RNA (e.g., mRNA). The sequences amplified in this manner form an “amplified region” or “amplicon.” Primer probes can be readily designed by those skilled in the art to target a specific template nucleic acid sequence.

By “analyte” is meant a substance or a constituent of a sample to be analyzed. Exemplary analytes include one or more species of one or more of the following: a nucleic acid, an oligonucleotide, RNA (e.g., mRNA), DNA, a protein, a peptide, a polypeptide, an amino acid, an antibody, a carbohydrate, a polysaccharide, glucose, a lipid, a gas (e.g., oxygen or carbon dioxide), an electrolyte (e.g., sodium, potassium, chloride, bicarbonate, BUN, magnesium, phosphate, calcium, ammonia, lactate), a lipoprotein, cholesterol, a fatty acid, a glycoprotein, a proteoglycan, a lipopolysaccharide, a cell surface marker (e.g., a cell surface protein of a Candida species (e.g., Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, and C. tropicalis)), a therapeutic agent, a metabolite of a therapeutic agent, an organism, a pathogen, a pathogen byproduct, a parasite (e.g., a protozoan or a helminth), a protist, a fungus (e.g., yeast (e.g., a Candida species (e.g., Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, and C. tropicalis)) or mold), a bacterium, an actinomycete, a cell (e.g., a whole cell, a white blood cell, a T cell (e.g., displaying CD3, CD4, CD8, IL2R, CD35, or other surface markers), or another cell identified with one or more specific markers), a virus, a prion, and components derived therefrom.

A “biological sample” is a sample obtained from a subject including but not limited to whole blood, serum, plasma, cerebrospinal fluid (CSF), urine, synovial fluid, breast milk, sweat, tears, saliva, semen, feces, vaginal fluid or tissue (e.g., from a vaginal swab), sputum, nasopharyngeal aspirate or swab, lacrimal fluid, mucous, or epithelial swab (e.g., a buccal swab, an axilla swab, a groin swab, an axilla/groin swab, or an ear swab), tissues (e.g., tissue homogenates), organs, bones, teeth, or culture media (e.g., BHI, SABHI, SDA, LB, and the like), among others. In some embodiments, the swab buffer diluent or swab transport medium is, without limitation, PBST, Amies Buffer, Amies Buffer+10% (v/v) 10×PBST, Cary Blair Media, or Liquid Stuart Swabs (which may include addition of 10% (v/v) 10×PBST). The biological sample may be a liquid sample.

As used herein, an “environmental sample” is a sample obtained from an environment, e.g., a surface swab sample, a sample from a building or a container, an air sample, a water sample, a soil sample, and the like. The environmental sample may contain any analyte described herein, e.g., a Candida species (e.g., Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, and/or C. tropicalis). In some embodiments, an environmental sample is from a hospital or other healthcare facility. In some embodiments, the environmental sample is a swab, e.g., swab buffer diluent or swab transport medium is, without limitation, PBST, Amies Buffer, Amies Buffer+10% (v/v) 10×PBST, Cary Blair Media, or Liquid Stuart Swabs (which may include addition of 10% (v/v) 10×PBST). The environmental sample may be a liquid sample.

As used herein, the term “small molecule” refers to a drug, medication, medicament, or other chemically synthesized compound that is contemplated for human therapeutic use.

A “biomarker” is a biological substance that can be used as an indicator of a particular disease state or particular physiological state of an organism, generally a biomarker is a protein or other native compound measured in bodily fluid whose concentration reflects the presence or severity or staging of a disease state or dysfunction, can be used to monitor therapeutic progress of treatment of a disease or disorder or dysfunction, or can be used as a surrogate measure of clinical outcome or progression.

By “an effective amount” is meant the amount of a composition (e.g., a pharmaceutical composition, e.g., a pharmaceutical composition that includes an antifungal agent) administered to improve, inhibit, or ameliorate a condition of a subject, or a symptom of a disorder or disease, e.g., Candidiasis, Candidemia, or sepsis, in a clinically relevant manner. Any improvement in the subject is considered sufficient to achieve treatment. Preferably, an amount sufficient to treat is an amount that reduces, inhibits, or prevents the occurrence or one or more symptoms of the disease or is an amount that reduces the severity of, or the length of time during which a subject suffers from, one or more symptoms of the disease (e.g., by at least 10%, 20%, or 30%, more preferably by at least 50%, 60%, or 70%, and most preferably by at least 80%, 90%, 95%, 99%, or more, relative to a control subject that is not treated with a composition). An effective amount of the composition used to practice the methods described herein varies depending upon the manner of administration and the age, body weight, and general health of the subject being treated. A physician or researcher can decide the appropriate amount and dosage regimen.

By an “isolated” nucleic acid molecule is meant a nucleic acid molecule that is removed from the environment in which it naturally occurs. For example, a naturally-occurring nucleic acid molecule present in the genome of cell or as part of a gene bank is not isolated, but the same molecule, separated from the remaining part of the genome, as a result of, e.g., a cloning event, amplification, or enrichment, is “isolated.” Typically, an isolated nucleic acid molecule is free from nucleic acid regions (e.g., coding regions) with which it is immediately contiguous, at the 5′ or 3′ ends, in the naturally occurring genome. Such isolated nucleic acid molecules can be part of a vector or a composition and still be isolated, as such a vector or composition is not part of its natural environment.

As used herein, “linked” means attached or bound by covalent bonds, non-covalent bonds, and/or linked via Van der Waals forces, hydrogen bonds, and/or other intermolecular forces.

The term “magnetic particle” refers to particles including materials of high positive magnetic susceptibility such as paramagnetic compounds, superparamagnetic compounds, and magnetite, gamma ferric oxide, or metallic iron.

As used herein, “nonspecific reversibility” refers to the colloidal stability and robustness of magnetic particles against non-specific aggregation in a liquid sample and can be determined by subjecting the particles to the intended assay conditions in the absence of a specific clustering moiety (i.e., an analyte or an agglomerator). For example, nonspecific reversibility can be determined by measuring the T₂ values of a solution of magnetic particles before and after incubation in a uniform magnetic field (defined as <5000 ppm) at 0.45 T for 3 minutes at 37° C. Magnetic particles are deemed to have nonspecific reversibility if the difference in T₂ values before and after subjecting the magnetic particles to the intended assay conditions vary by less than 10% (e.g., vary by less than 9%, 8%, 6%, 4%, 3%, 2%, or 1%). If the difference is greater than 10%, then the particles exhibit irreversibility in the buffer, diluents, and matrix tested, and manipulation of particle and matrix properties (e.g., coating and buffer formulation) may be required to produce a system in which the particles have nonspecific reversibility. In another example, the test can be applied by measuring the T₂ values of a solution of magnetic particles before and after incubation in a gradient magnetic field 1 Gauss/mm-10000 Gauss/mm.

As used herein, the term “NMR relaxation rate” refers to a measuring any of the following in a sample T₁, T₂, T₁/T₂ hybrid, T₁ᵣₕₒ, T₂ᵣₕₒ, and T₂*. The systems and methods of the invention are designed to produce an NMR relaxation rate characteristic of whether an analyte is present in the liquid sample. In some instances the NMR relaxation rate is characteristic of the quantity of analyte present in the liquid sample.

As used herein, the term “T₁/T₂ hybrid” refers to any detection method that combines a T₁ and a T₂ measurement. For example, the value of a T₁/T₂ hybrid can be a composite signal obtained through the combination of, ratio, or difference between two or more different T₁ and T₂ measurements. The T₁/T₂ hybrid can be obtained, for example, by using a pulse sequence in which T₁ and T₂ are alternatively measured or acquired in an interleaved fashion. Additionally, the T₁/T₂ hybrid signal can be acquired with a pulse sequence that measures a relaxation rate that is comprised of both T₁ and T₂ relaxation rates or mechanisms.

A “pathogen” means an agent causing disease or illness to its host, such as an organism or infectious particle, capable of producing a disease in another organism, and includes but is not limited to bacteria, viruses, protozoa, prions, yeast and fungi or pathogen by-products. A pathogen may be a Candida species, including Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, and C. tropicalis. “Pathogen by-products” are those biological substances arising from the pathogen that can be deleterious to the host or stimulate an excessive host immune response, for example pathogen antigen/s, metabolic substances, enzymes, biological substances, or toxins.

By “pathogen-associated analyte” is meant an analyte characteristic of the presence of a pathogen (e.g., a Candida species, including Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, and C. tropicalis) in a sample. The pathogen-associated analyte can be a particular substance derived from a pathogen (e.g., a protein, nucleic acid, lipid, polysaccharide, or any other material produced by a pathogen) or a mixture derived from a pathogen (e.g., whole cells, or whole viruses). In certain instances, the pathogen-associated analyte is selected to be characteristic of the genus, species, or specific strain of pathogen being detected. Alternatively, the pathogen-associated analyte is selected to ascertain a property of the pathogen, such as resistance to a particular therapy. In some embodiments, a pathogen-associated analyte may be a target nucleic acid that has been amplified. In other embodiments, a pathogen-associated analyte may be a host antibody or other immune system protein that is expressed in response to an infection by a Candida species such as Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, and C. tropicalis (e.g., an IgM antibody, an IgA antibody, an IgG antibody, or a major histocompatibility complex (MHC) protein).

By “pharmaceutical composition” is meant any composition that contains a therapeutically or biologically active agent (e.g., an antifungal agent) that is suitable for administration to a subject.

By “pulse sequence” or “RF pulse sequence” is meant one or more radio frequency pulses to be applied to a sample and designed to measure, e.g., certain NMR relaxation rates, such as spin echo sequences. A pulse sequence may also include the acquisition of a signal following one or more pulses to minimize noise and improve accuracy in the resulting signal value.

As used herein, the term “signal” refers to an NMR relaxation rate, frequency shift, susceptibility measurement, diffusion measurement, or correlation measurements.

As used herein, reference to the “size” of a magnetic particle refers to the average diameter for a mixture of the magnetic particles as determined by microscopy, light scattering, or other methods.

A “subject” is an animal, preferably a mammal (including, for example, rodents (e.g., mice or rats), farm animals (e.g., cows, sheep, horses, and donkeys), pets (e.g., cats and dogs), or primates (e.g., non-human primates and humans)). In particular embodiments, the subject is a human. A subject may be a patient (e.g., a patient having or suspected of having a disease such as Candidiasis or sepsis).

As used herein, the term “substantially monodisperse” refers to a mixture of magnetic particles having a polydispersity in size distribution as determined by the shape of the distribution curve of particle size in light scattering measurements. The FWHM (full width half max) of the particle distribution curve less than 25% of the peak position is considered substantially monodisperse. In addition, only one peak should be observed in the light scattering experiments and the peak position should be within one standard deviation of a population of known monodisperse particles.

By “T₂ relaxivity per particle” is meant the average T₂ relaxivity per particle in a population of magnetic particles.

As used herein, “unfractionated” refers to an assay in which none of the components of the sample being tested are removed following the addition of magnetic particles to the sample and prior to the NMR relaxation measurement.

As used herein, the term “cells/mL” indicates the number of cells per milliliter of a biological or environmental sample. The number of cells may be determined using any suitable method, for example, hemocytometer, quantitative PCR, and/or automated cell counting. It is to be understood that in some embodiments, cells/mL may indicate the number of colony-forming units per milliliter of a biological or environmental sample.

It is contemplated that units, methods, systems, and processes of the claimed invention encompass variations and adaptations developed using information from the embodiments described herein. Throughout the description, where units and systems are described as having, including, or including specific components, or where processes and methods are described as having, including, or including specific steps, it is contemplated that, additionally, there are units and systems of the present invention that consist essentially of, or consist of, the recited components, and that there are processes and methods according to the present invention that consist essentially of, or consist of, the recited processing steps. It should be understood that the order of steps or order for performing certain actions is immaterial, unless otherwise specified, so long as the invention remains operable. Moreover, in many instances, two or more steps or actions may be conducted simultaneously.

Magnetic Particles and NMR-Based Detection

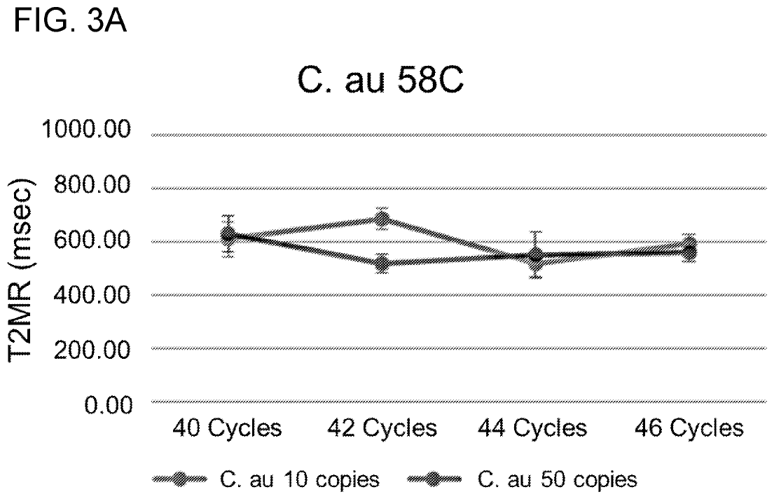

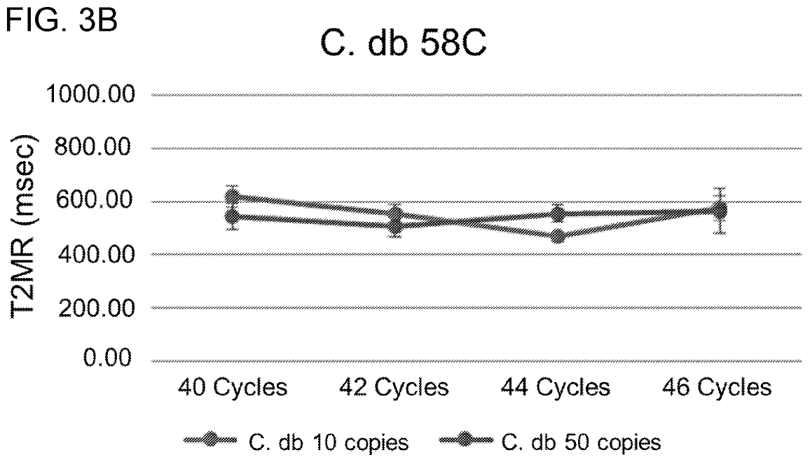

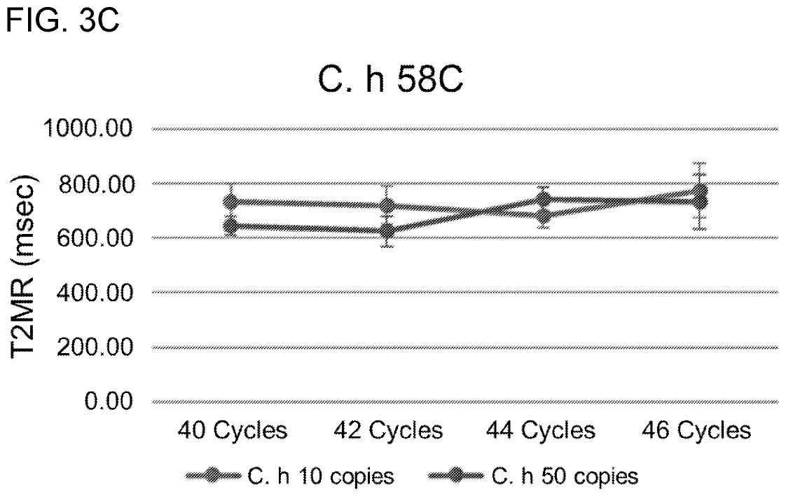

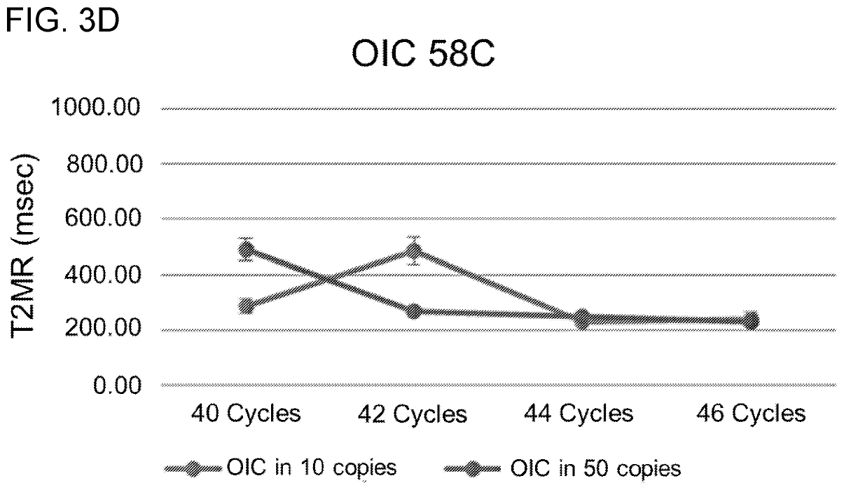

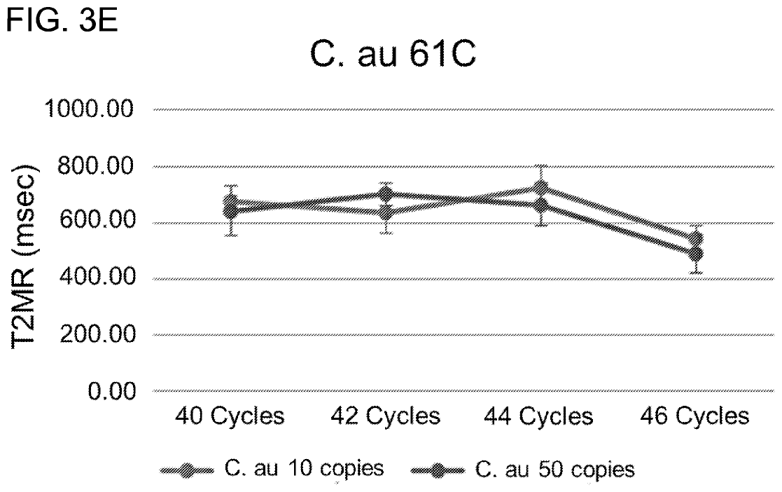

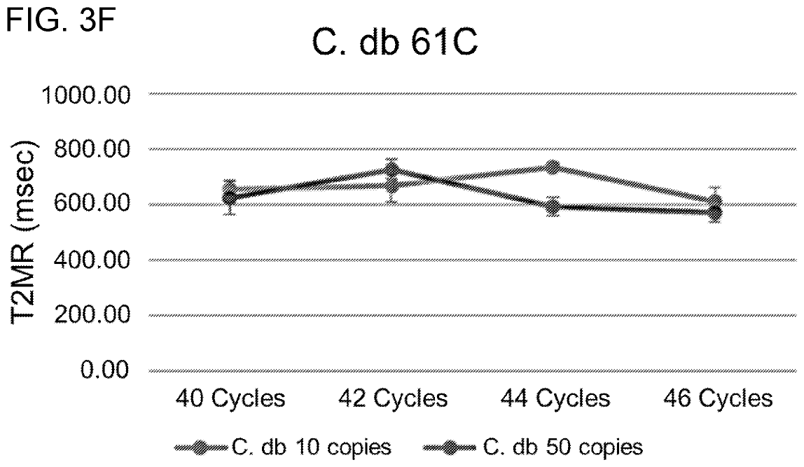

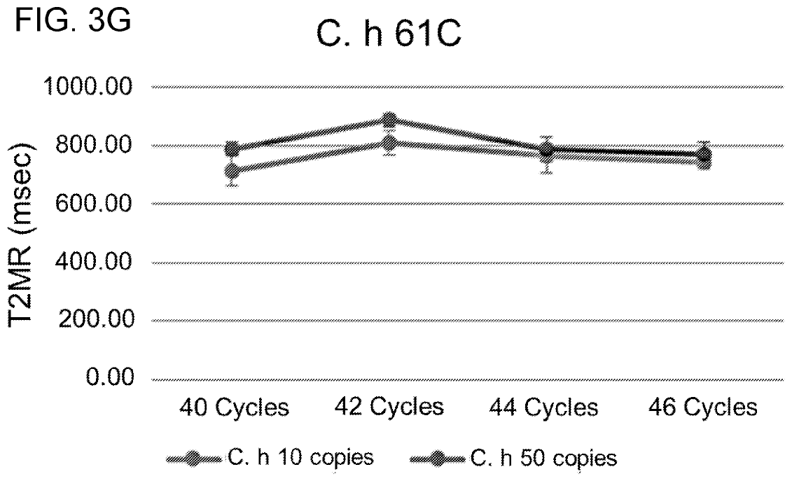

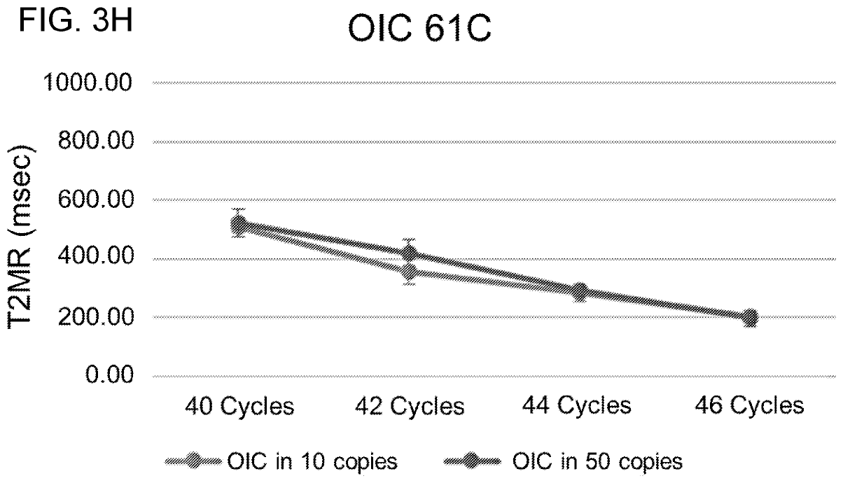

The methods and systems of the invention may involve use of magnetic particles and NMR. The magnetic particles can be coated with a binding moiety (e.g., oligonucleotide, antibody, etc.) such that in the presence of analyte, or multivalent binding agent, aggregates are formed. Aggregation depletes portions of the sample from the microscopic magnetic non-uniformities that disrupt the solvent's T₂ signal, leading to an increase in T₂ relaxation (see, e.g., FIG. 3 of International Patent Application Publication No. WO 2012/054639, which is incorporated herein by reference in its entirety).

The T₂ measurement is a single measure of all spins in the ensemble, measurements lasting typically 1-10 seconds, which allows the solvent to travel hundreds of microns, a long distance relative to the microscopic non-uniformities in the liquid sample. Each solvent molecule samples a volume in the liquid sample and the T₂ signal is an average (net total signal) of all (nuclear spins) on solvent molecules in the sample; in other words, the T₂ measurement is a net measurement of the entire environment experienced by a solvent molecule, and is an average measurement of all microscopic non-uniformities in the sample.

The observed T₂ relaxation rate for the solvent molecules in the liquid sample is dominated by the magnetic particles, which in the presence of a magnetic field form high magnetic dipole moments. In the absence of magnetic particles, the observed T₂ relaxation rates for a liquid sample are typically long (i.e., T₂ (water)=approximately 2000 ms, T₂ (blood)=approximately 1500 ms). As particle concentration increases, the microscopic non-uniformities in the sample increase and the diffusion of solvent through these microscopic non-uniformities leads to an increase in spin decoherence and a decrease in the T₂ value. The observed T₂ value depends upon the particle concentration in a non-linear fashion, and on the relaxivity per particle parameter.

In the aggregation assays of the invention, the number of magnetic particles, and, if present, the number of agglomerant particles, remain constant during the assay. The spatial distribution of the particles changes when the particles cluster. Aggregation changes the average “experience” of a solvent molecule because particle localization into clusters is promoted rather than more even particle distributions. At a high degree of aggregation, many solvent molecules do not experience microscopic non-uniformities created by magnetic particles and the T₂ approaches that of solvent. As the fraction of aggregated magnetic particles increases in a liquid sample, the observed T₂ is the average of the non-uniform suspension of aggregated and single (unaggregated) magnetic particles. The assays of the invention are designed to maximize the change in T₂ with aggregation to increase the sensitivity of the assay to the presence of analytes, and to differences in analyte concentration.

In some embodiments, the methods of the invention involve contacting a solution (e.g., a biological sample (e.g., whole blood) or an environmental sample) with between from 1×10⁶ to 1×10¹³ magnetic particles per milliliter of the liquid sample (e.g., from 1×10⁶ to 1×10⁸, 1×10⁷ to 1×10⁸, 1×10⁷ to 1×10⁹, 1×10⁸ to 1×10¹⁰, 1×10⁹ to 1×10¹¹, or 1×10¹⁰ to 1×10¹³ magnetic particles per milliliter).

In some embodiments, the magnetic particles used in the methods and systems of the invention have a mean diameter of from 150 nm to 1200 nm (e.g., from 150 to 250, 200 to 350, 250 to 450, 300 to 500, 450 to 650, 500 to 700 nm, 700 to 850, 800 to 950, 900 to 1050, or from 1000 to 1200 nm). For example, in some embodiments, the magnetic particles used in the methods of the invention may have a mean diameter of from 150 nm to 699 nm (e.g., from 150 to 250, 200 to 350, 250 to 450, 300 to 500, 450 to 650, or from 500 to 699 nm). In other embodiments, the magnetic particles used in the methods of the invention may have a mean diameter of from 700 nm to 1200 nm (e.g., from 700 to 850, 800 to 950, 900 to 1050, or from 1000 to 1200 nm). In particular embodiments, the magnetic particles may have a mean diameter of from 700 nm to 950 nm (e.g., from 700 to 750, 700 to 800, 700 to 850, or from 700 to 900 nm).

In some embodiments, the magnetic particles used in the methods of the invention may have a T₂ relaxivity per particle of from 1×10⁸ to 1×10¹² mM⁻¹s⁻¹ (e.g., from 1×10⁸ to 1×10⁹, 1×10⁸ to 1×10¹⁰, 1×10⁹ to 1×10¹⁰, 1×10⁹ to 1×10¹¹, or from 1×10¹⁰ to 1×10¹² mM⁻¹s⁻¹). In some embodiments, the magnetic particles have a T₂ relaxivity per particle of from 1×10⁹ to 1×10¹² mM⁻¹s⁻¹ (e.g., from 1×10⁹ to 1×10¹⁰, 1×10⁹ to 1×10¹¹, or from 1×10¹⁰ to 1×10¹² mM⁻¹s⁻¹).

In some embodiments, the magnetic particles may be substantially monodisperse. In some embodiments, the magnetic particles in a biological sample or an environmental sample (e.g., a liquid sample) may exhibit nonspecific reversibility in the absence of the one or more analytes and multivalent binding agent. In some embodiments, the magnetic particles may further include a surface decorated with a blocking agent selected from albumin, fish skin gelatin, gamma globulin, lysozyme, casein, peptidase, and an amine-bearing moiety (e.g., amino polyethyleneglycol, glycine, ethylenediamine, or amino dextran.

Analytes

Analytes may include or be derived from organisms such as Candida species, including Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, and C. tropicalis. For example, in some embodiments, the analyte may include or be derived from Candida auris, Candida lusitaniae, Candida haemulonii, Candida duobushaemulonii, or Candida pseudohaemulonii.

In some embodiments, the analyte may be an excreted or non-excreted (such as a surface antigen) protein expressed by any of the pathogens described above. In other embodiments, the analyte may be an antibody or other immune system protein that was expressed by the host in response to infection by any of the pathogens described herein (e.g., an IgM antibody, an IgA antibody, an IgG antibody, or a major histocompatibility complex (MHC) protein).

In some embodiments, the analyte may be a nucleic acid derived from any of the organisms described above. In some embodiments, the nucleic acid is a target nucleic acid derived from the organism that has been amplified. In some embodiments, the target nucleic acid may be a multi-copy locus. Use of a target nucleic acid derived from a multi-copy locus, in particular in methods involving amplification, may lead to an increase in sensitivity in the assay. Exemplary multi-copy loci may include, for example, ribosomal DNA (rDNA) operons, multi-copy plasmids, and the like. In other embodiments, the target nucleic acid may be a single-copy locus. In particular embodiments, the target nucleic acid may be derived from an essential locus, for example, an essential house-keeping gene. In particular embodiments, the target nucleic acid may be derived from a locus that is involved in virulence (e.g., a virulence gene). In any of the above embodiments, a locus may include a gene and/or an intragenic region.

Candida Target Nucleic Acids

In some embodiments, a target nucleic acid may include sequence elements that are specific for a Candida species (e.g., Candida auris, Candida lusitaniae, Candida haemulonii, Candida duobushaemulonii, Candida pseudohaemulonii, Candida albicans, Candida guilliermondii, Candida glabrata, Candida krusei, Candida lusitaniae, Candida parapsilosis, and Candida tropicalis). For example, in some embodiments, a Candida auris target nucleic acid may be amplified in the presence of a forward primer and a reverse primer which are specific to Candida auris. Detection of such a target nucleic acid in a sample would typically indicate that a Candida auris cell was present in the sample. In other embodiments, a target nucleic acid of the invention may include sequence elements that are common to all or a plurality of Candida species. For example, in some embodiments, a Candida species target nucleic acid may be amplified in the presence of a forward primer and a reverse primer, each of which is universal to all Candida species. In some embodiments, a Candida species target nucleic acid may be amplified in the presence of a forward primer and a reverse primer, each of which is universal to a plurality of Candida species (e.g., Candida auris, Candida lusitaniae, Candida haemulonii, Candida duobushaemulonii, and Candida pseudohaemulonii). Detection of such a target nucleic acid in a sample typically would indicate that a Candida species cell was present in the sample. In yet other embodiments, these approaches may be combined.

In some embodiments, a Candida species target nucleic acid may be derived from a linear chromosome or a linear or circular plasmid (e.g., a single-, low-, or multi-copy plasmid). In some embodiments, a Candida species target nucleic acid may be derived from an essential locus (e.g., an essential housekeeping gene) or a locus involved in virulence (e.g., a gene essential for virulence). In some embodiments, a Candida species target nucleic acid may be derived from a multi-copy locus. For example, in some embodiments, a Candida species target nucleic acid may be derived from a ribosomal DNA operon.

Detection of a Candida species can be performed as described, for example, in International Patent Application Publication No. WO 2012/054639, which is incorporated herein by reference in its entirety.

In some embodiments, a Candida species target nucleic acid may be amplified in the presence of a forward primer that includes the oligonucleotide sequence 5′-GGC ATG CCT GTT TGA GCG T-3′ (SEQ ID NO: 1) or GGG CAT GCC TGT TTG AGC GT-3′ (SEQ ID NO: 2) and a reverse primer that includes the oligonucleotide sequence 5′-GCT TAT TGA TAT GCT TAA GTT CAG CGG GT-3′ (SEQ ID NO: 3). For example, in some embodiments, a Candida species target nucleic acid may be amplified in the presence of a forward primer that includes the oligonucleotide sequence 5′-GGC ATG CCT GTT TGA GCG T-3′ (SEQ ID NO: 1) and a reverse primer that includes the oligonucleotide sequence 5′-GCT TAT TGA TAT GCT TAA GTT CAG CGG GT-3′ (SEQ ID NO: 3). In other embodiments, a Candida species target nucleic acid may be amplified in the presence of a forward primer that includes the oligonucleotide sequence GGG CAT GCC TGT TTG AGC GT-3′ (SEQ ID NO: 2) and a reverse primer that includes the oligonucleotide sequence 5′-GCT TAT TGA TAT GCT TAA GTT CAG CGG GT-3′ (SEQ ID NO: 3). The capture probes listed in Table 1 can be used for detection of an amplicon produced by these primers to identify the presence of the indicated Candida species. The dual target probe pair will detect either or both targets present in a sample.

TABLE 1
Capture Probes for Detection of Candida auris, Candida lusitaniae, Candida haemulonii,
Candida duobushaemulonii, and Candida pseudohaemulonii.

| Candida Capture Probes | Sequence |
|---|---|
| Candida auris 5′ Capture | CTA CCT GAT TTG AGG CGA CAA CAA AAC (SEQ ID NO: 4) |
| probe | |
| Candida auris 3′ Capture | CCG CGA AGA TTG GTG AGA AGA CAT (SEQ ID NO: 5) |
| probe | |
| Candida lusitaniae 5′ | CCT ACC TGA TTT GAG GGC GAA ATG TC (SEQ ID NO: 6) |
| Capture probe | |
| Candida lusitaniae 3′ | GGA GCA ACG CCT AAC CGG G (SEQ ID NO: 7) |
| Capture probe | |
| Candida haemulonii 5′ | GTC CTA CCT GAT TTG AGG GGA AAA AGC (SEQ ID NO: 8) |
| Capture probe | |
| Candida haemulonii 3′ | AAC AAA TCC ACC AAC GGT GAG AAG ATA T (SEQ ID NO: 9) |
| Capture probe | |
| Candida duobushaemulonii | GCG TAG ACT TCG CTG CGG AT (SEQ ID NO: 28) |
| 5′ Capture probe | |
| Alternate Candida | CGT AGA CTT CGC TGC GGA T (SEQ ID NO: 48) |
| duobushaemulonii 5′ | |
| Capture probe | |
| Candida duobushaemulonii | CTG GGC GGT GAG AAG AAA TC (SEQ ID NO: 29) |
| 3′ Capture probe | |
| Candida pseudohaemulonii | GCG TAG ACT TCG CTG CTG GAA (SEQ ID NO: 30) |
| 5′ Capture probe | |
| Candida pseudohaemulonii | CCG TGC GGT GAG AAG AAA TC (SEQ ID NO: 31) |
| 3′ Capture probe | |
| Candida duobushaemulonii | TCC TAC CTG ATT TGA GGA AAT AGC ATG G (SEQ ID NO: 32) |
| and Candida | |
| pseudohaemulonii (dual | |
| target) 5′ Capture probe | |
| Candida duobushaemulonii | ATT TAG CGG ATG CAA AAC CAC C (SEQ ID NO: 33) |
| and Candida | |
| pseudohaemulonii (dual | |
| target) 3′ Capture probe | |

In some methods, a Candida species amplicon produced by amplification of a Candida species target nucleic acid in the presence of a forward primer comprising the oligonucleotide sequence 5′-GGC ATG CCT GTT TGA GCG T-3′ (SEQ ID NO: 1) or 5′-GGG CAT GCC TGT TTG AGC GT-3′ (SEQ ID NO: 2) and a reverse primer that includes the oligonucleotide sequence 5′-GCT TAT TGA TAT GCT TAA GTT CAG CGG GT-3′ (SEQ ID NO: 3) is detected by hybridization a first nucleic acid probe and a second nucleic acid probe conjugated to one or more populations of magnetic particles. For example, in certain embodiments, (i) the Candida species is Candida auris, the first probe includes the oligonucleotide sequence 5′-CTA CCT GAT TTG AGG CGA CAA CAA AAC-3′ (SEQ ID NO: 4), and the second probe includes the oligonucleotide sequence 5′-CCG CGA AGA TTG GTG AGA AGA CAT-3′ (SEQ ID NO: 5); (ii) the Candida species is Candida lusitaniae, the first probe includes the oligonucleotide sequence 5′-CCT ACC TGA TTT GAG GGC GAA ATG TC-3′ (SEQ ID NO: 6), and the second probe includes the oligonucleotide sequence 5′-GGA GCA ACG CCT AAC CGG G-3′ (SEQ ID NO: 7); (iii) the Candida species is Candida haemulonii, the first probe includes the oligonucleotide sequence: 5′-GTC CTA CCT GAT TTG AGG GGA AAA AGC-3′ (SEQ ID NO: 8), and the second probe includes the oligonucleotide sequence: 5′-AAC AAA TCC ACC AAC GGT GAG AAG ATA T-3′ (SEQ ID NO: 9); (iv) the Candida species is Candida duobushaemulonii, the first probe includes the oligonucleotide sequence: 5′-CGT AGA CTT CGC TGC GGA T-3′ (SEQ ID NO: 48) or 5′-GCG TAG ACT TCG CTG CGG AT-3′ (SEQ ID NO: 28), and the second probe includes the oligonucleotide sequence: 5′-CTG GGC GGT GAG AAG AAA TC-3′ (SEQ ID NO: 29); (v) the Candida species is Candida pseudohaemulonii, the first probe includes the oligonucleotide sequence: 5′-GCG TAG ACT TCG CTG CTG GAA-3′ (SEQ ID NO: 30), and the second probe includes the oligonucleotide sequence: 5′-CCG TGC GGT GAG AAG AAA TC-3′ (SEQ ID NO: 31); and/or (vi) the Candida species is Candida duobushaemulonii or Candida pseudohaemulonii, the first probe includes the oligonucleotide sequence: 5′-TCC TAC CTG ATT TGA GGA AAT AGC ATG G-3′ (SEQ ID NO: 32), and the second probe includes the oligonucleotide sequence: 5′-ATT TAG CGG ATG CAA AAC CAC C-3′ (SEQ ID NO: 33).

In other embodiments, a Candida species target nucleic acid may be amplified in the presence of a forward primer that includes the oligonucleotide sequence 5′-GGC ATG CCT GTT TGA GCG T-3′ (SEQ ID NO: 1) or 5′-GGC ATG CCT GTT TGA GCG TC-3′ (SEQ ID NO: 10) and a reverse primer that includes the oligonucleotide sequence 5′-GCT TAT TGA TAT GCT TAA GTT CAG CGG GT-3′ (SEQ ID NO: 3). The capture probes listed in Table 2 can be used for detection of an amplicon produced by these primers to identify the presence of the indicated Candida species.

TABLE 2
Capture Probes for Detection of additional Candida species.

| Candida Capture Probes | Sequence |
|---|---|
| Candida albicans Probe #1 | ACC CAG CGG TTT GAG GGA GAA AC (SEQ ID NO: 11) |
| Candida albicans Probe #2 | AAA GTT TGA AGA TAT ACG TGG TGG ACG TTA (SEQ ID NO: 12) |
| Candida krusei Probe #1 | CGC ACG CGC AAG ATG GAA ACG (SEQ ID NO: 13) |
| Candida krusei Probe #2 | AAG TTC AGC GGG TAT TCC TAC CT (SEQ ID NO: 14) |
| Candida krusei probe | AGC TTT TTG TTG TCT CGC AAC ACT CGC (SEQ ID NO: 15) |
| Candida glabrata Probe #1 | CTA CCA AAC ACA ATG TGT TTG AGA AG (SEQ ID NO: 16) |
| Candida glabrata Probe #2 | CCT GAT TTG AGG TCA AAC TTA AAG ACG TCT G (SEQ ID NO: 17) |
| Candida | AGT CCT ACC TGA TTT GAG GTC NitInd¹ AA (SEQ ID NO: 18) |
| parapsilosis/tropicalis Probe | |
| #1 | |
| Candida | CCG NitInd¹GG GTT TGA GGG AGA AAT (SEQ ID NO: 19) |
| parapsilosis/tropicalis Probe | |
| #2 | |
| Candida tropicalis | AAA GTT ATG AAATAA ATT GTG GTG GCC ACT AGC (SEQ ID NO: |
| | 20) |
| Candida tropicalis | ACC CGG GGGTTT GAG GGA GAA A (SEQ ID NO: 21) |
| Candida parapsilosis | AGT CCT ACC TGA TTT GAG GTC GAA (SEQ ID NO: 22) |
| Candida parapsilosis | CCG AGG GTT TGA GGG AGA AAT (SEQ ID NO: 23) |

¹NitInd is 5′ 5-Nitroindole, a base that is capable of annealing with any of the four DNA bases.

In some methods, a Candida species amplicon produced by amplification of a Candida species target nucleic acid in the presence of a forward primer comprising the oligonucleotide sequence 5′-GGC ATG CCT GTT TGA GCG T-3′ (SEQ ID NO: 1) or 5′-GGC ATG CCT GTT TGA GCG TC-3′ (SEQ ID NO: 10) and a reverse primer that includes the oligonucleotide sequence 5′-GCT TAT TGA TAT GCT TAA GTT CAG CGG GT-3′ (SEQ ID NO: 3) is detected by hybridization a first nucleic acid probe and a second nucleic acid probe conjugated to one or more populations of magnetic particles. For example, certain embodiments, (i) the Candida species is Candida albicans, the first probe includes the oligonucleotide sequence 5′-ACC CAG CGG TTT GAG GGA GAA AC-3′ (SEQ ID NO: 11), and the second probe includes the oligonucleotide sequence 5′-AAA GTT TGA AGA TAT ACG TGG TGG ACG TTA-3′ (SEQ ID NO: 12); (ii) the Candida species is Candida krusei and the first probe and the second probe include an oligonucleotide sequence selected from: 5′-CGC ACG CGC AAG ATG GAA ACG-3′ (SEQ ID NO: 13), 5′-AAG TTC AGC GGG TAT TCC TAC CT-3′ (SEQ ID NO: 14), and 5′-AGC TTT TTG TTG TCT CGC AAC ACT CGC-3′ (SEQ ID NO: 15); (iii) the Candida species is Candida glabrata, the first probe includes the oligonucleotide sequence: 5′-CTA CCA AAC ACA ATG TGT TTG AGA AG-3′ (SEQ ID NO: 16), and the second probe includes the oligonucleotide sequence: 5′-CCT GAT TTG AGG TCA AAC TTA AAG ACG TCT G-3′ (SEQ ID NO: 17); and (iv) the Candida species is Candida parapsilosis or Candida tropicalis and the first probe and the second probe include an oligonucleotide sequence selected from: 5′-AGT CCT ACC TGA TTT GAG GTCNitIndAA-3′ (SEQ ID NO: 18), 5′-CCG NitIndGG GTT TGA GGG AGA AAT-3′ (SEQ ID NO: 19), 5′-AAA GTT ATG AAATAA ATT GTG GTG GCC ACT AGC-3′ (SEQ ID NO: 20), 5′-ACC CGG GGGTTT GAG GGA GAA A-3′ (SEQ ID NO: 21), 5′-AGT CCT ACC TGA TTT GAG GTC GAA-3′ (SEQ ID NO: 22), and 5′-CCG AGG GTT TGA GGG AGA AAT-3′ (SEQ ID NO: 23).

Variant Primers and Probes

In some embodiments, the invention features a primer that has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any of the preceding forward or reverse primers. For example, in some embodiments, the invention features a forward primer comprising an oligonucleotide sequence that is at least 80% identical (e.g., at least 80%, 81%, 82%, 83%, 84%, 85%, 86%, 87%, 88%, 89%, 90%, 91%, 92%, 93%, 94%, 95%, 96%, 97%, 98%, 99%, or 100% identical) to any one of SEQ ID NOs: 1, 2, or 10. In some embodiments, the invention features a reverse primer comprising an oligonucleotide sequence that is at least 80% identical (e.g., at least 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO: 3. Such primers can be used in any of the methods of the invention described herein.

In some embodiments, the invention features a probe that has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, 99%, or 100%) identity with any of the preceding probes. For example, in some embodiments, the invention features a 5′ capture probe comprising an oligonucleotide sequence that is at least 80% identical (e.g., at least 80%, 81%, 82%, 83%, 84%, 85%, 86%, 87%, 88%, 89%, 90%, 91%, 92%, 93%, 94%, 95%, 96%, 97%, 98%, 99%, or 100% identical) to any one of SEQ ID NOs: 4, 6, 8, 28, 30, 32, or 48. In some embodiments, the invention features a 3′ capture probe comprising an oligonucleotide sequence that is at least 80% identical (e.g., at least 80%, 81%, 82%, 83%, 84%, 85%, 86%, 87%, 88%, 89%, 90%, 91%, 92%, 93%, 94%, 95%, 96%, 97%, 98%, 99%, or 100% identical) to any one of SEQ ID NOs: 5, 7, 9, 29, 31, or 33. Such probes can be used in any of the methods of the invention described herein.

In some embodiments, any of the preceding primers or probes may include one or more modified bases, for example, 2,6-Diaminopurine (abbreviated herein as “/i6diPr/”), deoxyinosine (abbreviated herein as “/ideoxyl/”), nitroindole (abbreviated herein as/35NiTInd/or NitInd) or other modified bases known in the art.

Medical Conditions

The methods and systems of the invention can also be used to diagnose and/or monitor an infectious disease. In some embodiments, the methods of the invention may be used to monitor and diagnose infectious disease in a multiplexed, automated, no sample preparation system. Examples of pathogens that may be detected using the methods of the invention include, e.g., Candida species, including Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, and C. tropicalis.

Exemplary diseases that can be diagnosed and/or monitored by the methods and systems of the invention include diseases caused by or associated with Candida species, including Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, and C. tropicalis (e.g., Candida infection (also known as Candidiasis), bloodstream infection (e.g., Candidemia), pneumonia, peritonitis, osteomyeletis, meningitis, empyema, urinary tract infection, sepsis, septic shock, and septic arthritis) and diseases that may manifest with similar symptoms to diseases caused by or associated with microbial pathogens such as Candida species (e.g., SIRS).

The methods and systems of the invention can be used to identify and monitor the pathogenesis of disease in a subject, to select therapeutic interventions, and to monitor the effectiveness of the selected treatment. For example, for a patient having or at risk of a disease (e.g., Candidiasis, Candidemia, or sepsis), the systems and methods of the invention can be used to identify the infectious pathogen, pathogen load, and to monitor white blood cell count and/or biomarkers indicative of the status of the infection. The identity of the pathogen can be used to select an appropriate therapy. In some embodiments, the methods may further include administering a therapeutic agent following monitoring or diagnosing an infectious disease. The therapeutic intervention (e.g., a particular antibiotic agent) can be monitored as well to correlate the treatment regimen to the circulating concentration of antibiotic agent and pathogen load to ensure that the patient is responding to treatment. In some embodiments, antimicrobial resistance markers (e.g., antimicrobial resistance genes) may be monitored following therapeutic intervention.

In certain embodiments, the methods and systems can distinguish whether a disease is caused by Candida auris, Candida lusitaniae, Candida haemulonii, Candida duobushaemulonii, or Candida pseudohaemulonii; by another Candida species (e.g., Candida albicans, Candida guilliermondii, Candida glabrata, Candida krusei, Candida lusitaniae, Candida parapsilosis, and Candida tropicalis; or by a non-Candida microbial pathogen. In some embodiments, the non-Candida microbial pathogen is a bacterial pathogen, including Gram-positive bacteria (e.g., Gram-positive anaerobic bacteria), Gram-negative bacteria (e.g., Gram-negative anaerobic bacteria), Enterobacteriaceae spp., Acinetobacter spp. (e.g., Acinetobacter baumannii), Enterococcus spp. (e.g., Enterococcus faecium and Enterococcus faecalis), Klebsiella spp. (e.g., Klebsiella pneumoniae), Pseudomonas spp. (e.g., Pseudomonas aeruginosa), Staphylococcus spp. (including, e.g., coagulase-positive species (e.g., Staphylococcus aureus) and coagulase-negative (CoNS) species), Streptococcus spp. (e.g., β-hemolytic streptococci, Streptococcus mitis, Streptococcus pneumoniae, Streptococcus agalactiae, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serratia marcescens), Citrobacter spp. (e.g., Citrobacter freundii), Enterobacter spp. (e.g., Enterobacter aerogenes and Enterobacter cloacae), Borrelia spp. (e.g., Borrelia burgdorferi, Borrelia afzelii, and Borrelia garinii), Rickettsia spp. (e.g., Rickettsia rickettsii), Anaplasma spp. (e.g., Anaplasma phagocytophilum), Coxiella spp. (e.g., Coxiella burnetii), Ehrlichia spp. (e.g., Ehrlichia chaffeensis and Ehrlichia ewingii), Franciscella spp. (e.g., Francisella tularensis), Clostridium spp. (e.g., Clostridium botulinum, Clostridium difficile, Clostridium perfringens, and Clostridium tetani), Bacteroides spp. (e.g., Bacteroides fragilis), and Neisseria spp. (e.g., Neisseria meningitides). In other embodiments, the non-Candida microbial pathogen is a fungal pathogen, e.g., Saccharomyces spp. (e.g., Saccharomyces cerevisiae), Aspergillus spp. (e.g., Aspergillus fumigatus, Aspergillus clavatus, and Aspergillus flavus), and Cryptococcus spp. (e.g., Cryptococcus neoformans, Cryptococcus laurentii, and Cryptococcus albidus). In yet other embodiments, the non-Candida microbial pathogen is a protozoan pathogen, including Babesia spp. (e.g., Babesia microti and Babesia divergens).

Treatment

The invention features methods of treating a patient suffering from a disease (e.g., Candidiasis, Candidemia, or sepsis). In some embodiments, the methods further include administering a therapeutic agent to a subject following a diagnosis. Typically, the identification of a particular pathogen will guide the selection of the appropriate therapeutic agent.

In some embodiments, the methods and systems of the invention can be used for rapid identification of patients for clinical studies. In typical drug development for anti-infective agents (e.g., antifungal agents), patients are recruited based on clinical presentation, not diagnostic data. Challenges include large clinical trials, a limited incidence of disease for the targeted pathogen, long development timeline, the fact that patients are already on empiric therapies, and the high blood culture false negative rate reduces enrollment of relevant patients. Using the methods and systems of the present invention, patients suffering from an infection (e.g., a Candida infection, such as by Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, or C. tropicalis, can rapidly and accurately be identified in less than 5 hours. Thus, a patient can be selected for a clinical trial for a therapeutic agent under investigation (i.e., a clinical trial) after testing positive for the presence of a Candida species (e.g., Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, or C. tropicalis). Such patient selection can decrease the size of trials by enriching for the right patient population, speed the timeline of clinical trials, lower the costs of clinical trials, and achieve clinical superiority (e.g., because patients are not treated with courses of empiric therapy).

This approach also has benefits for commercialization for targeted anti-infective compounds, which are typically competing against low cost, broad spectrum solutions, with short term and non-recurring use. This approach has major benefits for patients; notably, improved outcome, such as improved mortality from early treatment with appropriate therapy, more rapid and effective treatment at the point of care, and reduced toxicity and exposure to unnecessary therapy. There are also benefits to hospitals, notably, improved patient outcomes, cost savings, reducing the length of stay, decrease in inappropriate therapy, and reduction of antimicrobial resistance thanks to use of appropriate targeted therapies. Benefits to pharmaceutical companies developing drugs can include clear market differentiation (e.g., increased effectiveness, faster approval process, lower cost and more efficient trials (enabling drug salvage and unique indications), and enhanced pricing of the drug).

The methods and systems of the invention can be used for antifungal stewardship, which is the judicious use of currently available antifungal agents to minimize development of antifungal resistance. For example, using the methods and systems of the invention, the infective pathogen (e.g., Candida species, such as Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, or C. tropicalis) can rapidly and accurately be determined in a patient's sample. This enables selection of appropriate targeted therapies that are likely improve treatment outcome instead of agents that are unlikely to improve treatment outcome and result in increased antifungal resistance.

In some embodiments, the treatment method may involve administration of an antifungal agent, for example, for treatment of a fungal (e.g., Candida) infection, e.g., Candidiasis or Candidemia. Exemplary antifungal agents suitable for use in the invention include, but are not limited to, 1,3-β-D-glucan synthesis inhibitors (e.g., caspofungin, anidulafungin, micafungin, enfumafungin, and SCY-078), polyenes (e.g., amphotericin B, candicidin, filipin, hamycin, natamycin, nystatin, and rimocidin), azoles (e.g., imidazoles such as bifonazole, butoconazole, clotrimazole, eberconazole, econazole, fenticonazole, flutrimazole, isoconazole, ketoconazole, luliconazole, miconazole, omoconazole, oxiconazole, sertaconazole, sulconazole, tioconazole; triazoles such as albaconazole, efinaconazole, epoxiconazole, fluconazole, isavuconazole, itraconazole, posaconazole, propiconazole, ravuconazole, terconazole, voriconazole; and thiazoles such as abafungin), allylamines (e.g., amorolfin, butenafine, naftifine, and terbinafine), echinocandins (e.g., anidulafungin, caspofungin, and micafungin), and other antifungal agents including but not limited to benzoic acid, ciclopirox olamine, 5-flucytosin, griseofulvin, haloprogin, tolnaftate, aminocandin, chlordantoin, chlorphenesin, nifuroxime, undecylenic acid, crystal violet and pharmaceutically acceptable salts or esters thereof.

For example, in particular embodiments, the invention features a method of treating a subject suffering from a disease that includes administering a therapeutic agent (e.g., an antifungal agent) to the subject, wherein the subject has been diagnosed as having the disease based on detecting the presence of a Candida species according to any of the methods described herein. In some instances, the Candida species is Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, or C. tropicalis. In some instances, the disease is Candidiasis, Candidemia, or sepsis. In some instances, the subject has a Candida auris infection and the antifungal agent is a 1,3-β-D-glucan synthesis inhibitor (e.g., caspofungin, anidulafungin, micafungin, enfumafungin, or SCY-078). In some instances, the 1,3-β-D-glucan synthesis inhibitor is SCY-078 (see, e.g., Larkin et al. Antimicrob. Agents Chemother. doi:10.1128/AAC.02396-16, 2017).

An antifungal agent or any other therapeutic agent may be administered by any suitable route. In some embodiments, an antifungal agent or any other therapeutic agent, or a pharmaceutical composition thereof, are administered by one or more of a variety of routes, including parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. However, the present disclosure encompasses the delivery of compositions of the invention by any appropriate route taking into consideration likely advances in the sciences of drug delivery. In general, the most appropriate route of administration will depend upon a variety of factors including the nature of the pharmaceutical composition (e.g., its stability in various bodily environments such as the bloodstream and gastrointestinal tract), and the condition of the patient (e.g., whether the patient is able to tolerate particular routes of administration).

A dose of an antifungal agent or any other therapeutic agent may be administered at any suitable frequency, in the same or a different amount, to obtain a desired drug concentration and/or effect (e.g., a therapeutic effect). The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). The specific therapeutically effective, prophylactically effective, or otherwise appropriate dose level for any particular subject will depend upon a variety of factors including the severity and identify of a disorder being treated, if any; the antimicrobial agent and/or other therapeutic agent employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

In some embodiments, an antifungal agent or other therapeutic agent, or a pharmaceutical composition thereof, may be administered in combination with another agent, for example, another therapeutic agent, a prophylactic agent, and/or a diagnostic agent. By “in combination with,” it is not intended to imply that the agents must be administered at the same time and/or formulated for delivery together, although these methods of delivery are within the scope of the present disclosure. For example, one or more compositions including one or more different antimicrobial agents may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the invention, or imaging, diagnostic, or prophylactic compositions thereof in combination with agents that improve their bioavailability, reduce and/or modify their metabolism, inhibit their excretion, and/or modify their distribution within the body.

Assay Reagents

The assays described herein may include any suitable reagents, for example, surfactants, buffer components, additives, chelating agents, and the like. The surfactant may be selected from a wide variety of soluble non-ionic surface active agents including surfactants that are generally commercially available under the IGEPAL trade name from GAF Company. The IGEPAL liquid non-ionic surfactants are polyethylene glycol p-isooctylphenyl ether compounds and are available in various molecular weight designations, for example, IGEPAL CA720, IGEPAL CA630, and IGEPAL CA890. Other suitable non-ionic surfactants include those available under the trade name TETRONIC 909 from BASF Wyandotte Corporation. This material is a tetra-functional block copolymer surfactant terminating in primary hydroxyl groups. Suitable non-ionic surfactants are also available under the VISTA ALPHONIC trade name from Vista Chemical Company and such materials are ethoxylates that are non-ionic biodegradables derived from linear primary alcohol blends of various molecular weights. The surfactant may also be selected from poloxamers, such as polyoxyethylene-polyoxypropylene block copolymers, such as those available under the trade names Synperonic PE series (ICI), PLURONIC® series (BASF), Supronic, Monolan, Pluracare, and Plurodac, polysorbate surfactants, such as TWEEN® 20 (PEG-20 sorbitan monolaurate), and glycols such as ethylene glycol and propylene glycol.

Such non-ionic surfactants may be selected to provide an appropriate amount of detergency for an assay without having a deleterious effect on assay reactions. In particular, surfactants may be included in a reaction mixture for the purpose of suppressing non-specific interactions among various ingredients of the aggregation assays of the invention. The non-ionic surfactants are typically added to the liquid sample prior in an amount from 0.01% (w/w) to 5% (w/w).

The non-ionic surfactants may be used in combination with one or more proteins (e.g., albumin, fish skin gelatin, lysozyme, or transferrin) also added to the liquid sample prior in an amount from 0.01% (w/w) to 5% (w/w).

Furthermore, the assays, methods, and cartridge units of the invention can include additional suitable buffer components (e.g., Tris base, selected to provide a pH of about 7.8 to 8.2 in the reaction milieu); and chelating agents to scavenge cations (e.g., ethylene diamine tetraacetic acid (EDTA), EDTA disodium, citric acid, tartaric acid, glucuronic acid, saccharic acid or suitable salts thereof).

Sample Preparation and Cell Lysis

The methods and systems of the invention may involve sample preparation and/or cell lysis. For example, a pathogen (e.g., a Candida species (e.g., Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, or C. tropicalis)) present in a biological or environmental sample may be lysed prior to amplification of a target nucleic acid. Suitable lysis methods for lysing pathogen cells in a biological sample (e.g., whole blood, cerebrospinal fluid (CSF), urine, synovial fluid, an epithelial swab (e.g., a buccal swab, an axilla swab, a groin swab, or an axilla/groin swab), or an environmental sample (e.g., an environmental swab, e.g., a surface swab)) include, for example, mechanical lysis (e.g., beadbeating and sonication), heat lysis, and alkaline lysis. In some embodiments, the swab buffer diluent or swab transport medium is, without limitation, PBST, Amies Buffer, Amies Buffer+10% (v/v) 10×PBST, Cary Blair Media, or Liquid Stuart Swabs (which may include addition of 10% (v/v) 10×PBST). In some embodiments, beadbeating may be performed by adding glass beads (e.g., 0.5 mm glass beads) to a biological or environmental sample to form a mixture and agitating the mixture. As an example, the sample preparation and cell lysis (e.g., beadbeating) may be performed using any of the approaches and methods described in WO 2012/054639.

In some embodiments, the methods of the invention involve detection of one or more pathogen-associated analytes in a whole blood sample. In some embodiments, the methods may involve disruption of red blood cells (erythrocytes). In some embodiments, the disruption of the red blood cells can be carried out using an erythrocyte lysis agent (i.e., a lysis buffer, an isotonic lysis agent, or a nonionic detergent). Erythrocyte lysis buffers which can be used in the methods of the invention include, without limitation, isotonic solutions of ammonium chloride (optionally including carbonate buffer and/or EDTA), and hypotonic solutions. The basic mechanism of hemolysis using isotonic ammonium chloride is by diffusion of ammonia across red blood cell membranes. This influx of ammonium increases the intracellular concentration of hydroxyl ions, which in turn reacts with CO₂ to form hydrogen carbonate. Erythrocytes exchange excess hydrogen carbonate with chloride which is present in blood plasma via anion channels and subsequently increase in intracellular ammonium chloride concentrations. The resulting swelling of the cells eventually causes loss of membrane integrity.

Alternatively, the erythrocyte lysis agent can be an aqueous solution of nonionic detergents (e.g., nonyl phenoxypolyethoxylethanol (NP-40), 4-octylphenol polyethoxylate (TRITON® X-100), BRIJ®-58, or related nonionic surfactants, and mixtures thereof). The erythrocyte lysis agent disrupts at least some of the red blood cells, allowing a large fraction of certain components of whole blood (e.g., certain whole blood proteins) to be separated (e.g., as supernatant following centrifugation) from the white blood cells or other cells (e.g., bacterial cells and protozoan cells) present in the whole blood sample. Following erythrocyte lysis and centrifugation, the resulting pellet may be reconstituted to form an extract.

In some embodiments, the methods of the invention may include (a) providing a whole blood sample from a subject; (b) mixing the whole blood sample with an erythrocyte lysis agent solution to produce disrupted red blood cells; (c) following step (b), centrifuging the sample to form a supernatant and a pellet, discarding some or all of the supernatant, and resuspending the pellet to form an extract, (d) lysing cells of the extract (which may include white blood cells and/or pathogen cells) to form a lysate. In some embodiments, the method further comprises amplifying one or more target nucleic acids (e.g., a Candida species target nucleic acid, a Candida auris target nucleic acid, a Candida lusitaniae target nucleic acid, a Candida haemulonii target nucleic acid, a Candida duobushaemulonii target nucleic acid, a Candida pseudohaemulonii target nucleic acid, a Candida guilliermondii target nucleic acid, a Candida albicans target nucleic acid, a Candida glabrata target nucleic acid, a Candida krusei target nucleic acid, a C. parapsilosis target nucleic acid, or a C. tropicalis target nucleic acid) in the lysate. In some embodiments, the method may include washing the pellet (e.g., with a buffer such as TE buffer) prior to resuspending the pellet and optionally repeating step (c). In some embodiments, the method may include 1, 2, 3, 4, 5, or more wash steps. In other embodiments, the method is performed without performing any wash step.

In some embodiments, the method includes: (a) contacting a whole blood sample suspected of containing one or more pathogen cells (e.g., a Candida species (e.g., Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, and/or C. tropicalis)) with an erythrocyte lysis agent, thereby lysing red blood cells; (b) centrifuging the product of step (a) to form a supernatant and a pellet; (c) discarding some or all of the supernatant of step (b) and washing the pellet once; (d) centrifuging the product of step (c) to form a supernatant and a pellet; (e) discarding some or all of the supernatant of step (d) and mixing the pellet of (d) with a buffer; (f) combining the product of step (e) with beads to form a mixture and agitating the mixture to form a lysate, said lysate containing both subject cell nucleic acid and pathogen nucleic acid; and (g) providing the lysate of step (f) in a detection tube and amplifying pathogen nucleic acids therein (e.g., by PCR) to form an amplified lysate solution; wherein ten pathogen cells per milliliter of the whole blood sample is sufficient to permit amplification of the target pathogen nucleic acid. In some embodiments, the amplified pathogen nucleic acid(s) are detected by measuring the T₂ relaxation response of the biological or environmental sample or a portion thereof following contacting the biological or environmental sample or the portion thereof with magnetic particles, wherein the magnetic particles have binding moieties on their surfaces, the binding moieties operative to alter the specific aggregation of the magnetic particles in the presence of the amplified amplified pathogen nucleic acid(s). In some embodiments, the method further comprises detecting the amplified pathogen nucleic acid(s), e.g., by sequencing (e.g., Sanger sequencing or high-throughput sequencing (e.g., ILLUMINA® sequencing), optical, fluorescent, mass, density, magnetic, chromatographic, and/or electrochemical measurement.

In some embodiments of any of the preceding methods, the washing of step (c) involves adding a buffer (e.g., TE buffer). In some embodiments, the buffer has a volume of about 20 μL, about 40 μL, about 60 μL, about 80 μL, about 100 μL, about 120 μL, about 140 μL, about 160 μL, about 180 μL, about 200 μL, about 220 μL, about 240 μL, about 260 μL, about 280 μL, about 300 μL, about 400 μL, about 500 μL, or more. In some embodiments, the buffer has a volume of about 100 μL or less, about 110 μL or less, about 120 μL or less, about 130 μL or less, about 140 μL or less, about 150 μL or less, about 160 μL or less, about 170 μL or less, about 180 μL or less, about 190 μL or less, about 200 μL or less, about 225 μL or less, about 250 μL or less, about 275 μL or less, about 300 μL or less, about 400 μL or less, about 500 μL or less, about 600 μL or less, about 700 μL or less, about 800 μL or less, about 900 μL or less, or about 1000 μL or less. For example, in some embodiments, the buffer has a volume of about 1 μL to about 200 μl, about 1 μL to about 175 μl, about 1 μL to about 150 μl, about 1 μL to about 125 μl, about 1 μL to about 100 μl, about 1 μL to about 75 μl, about 1 μL to about 50 μl, about 1 μL to about 25 μl, about 25 μL to about 200 μl, about 25 μL to about 175 μl, about 25 μL to about 150 μl, about 25 μL to about 125 μl, about 25 μL to about 100 μl, about 25 μL to about 75 μl, about 25 μL to about 50 μl, about 50 μL to about 200 μl, about 50 μL to about 175 μl, about 50 μL to about 150 μl, about 50 μL to about 125 μl, about 50 μL to about 100 μl, about 50 μL to about 75 μl, about 75 μL to about 200 μl, about 75 μL to about 175 μl, about 75 μL to about 150 μl, about 75 μL to about 125 μl, or about 75 μL to about 100 μl.

Any suitable buffer may be used in step (e) in any of the preceding methods. For example, in some embodiments, the buffer of step (e) is TE buffer. In some embodiments, the buffer (e.g., TE buffer) of step (e) has a volume of about 20 μL, about 40 μL, about 60 μL, about 80 μL, about 100 μL, about 120 μL, about 140 μL, about 160 μL, about 180 μL, about 200 μL, about 220 μL, about 240 μL, about 260 μL, about 280 μL, about 300 μL, about 400 μL, about 500 μL, or more. In some embodiments, the buffer of step (e) has a volume of about 100 μL or less, about 110 μL or less, about 120 μL or less, about 130 μL or less, about 140 μL or less, about 150 μL or less, about 160 μL or less, about 170 μL or less, about 180 μL or less, about 190 μL or less, about 200 μL or less, about 225 μL or less, about 250 μL or less, about 275 μL or less, about 300 μL or less, about 400 μL or less, about 500 μL or less, about 600 μL or less, about 700 μL or less, about 800 μL or less, about 900 μL or less, or about 1000 μL or less. For example, in some embodiments, the buffer of step (e) has a volume of about 1 μL to about 200 μl, about 1 μL to about 175 μl, about 1 μL to about 150 μl, about 1 μL to about 125 μl, about 1 μL to about 100 μl, about 1 μL to about 75 μl, about 1 μL to about 50 μl, about 1 μL to about 25 μl, about 25 μL to about 200 μl, about 25 μL to about 175 μl, about 25 μL to about 150 μl, about 25 μL to about 125 μl, about 25 μL to about 100 μl, about 25 μL to about 75 μl, about 25 μL to about 50 μl, about 50 μL to about 200 μl, about 50 μL to about 175 μl, about 50 μL to about 150 μl, about 50 μL to about 125 μl, about 50 μL to about 100 μl, about 50 μL to about 75 μl, about 75 μL to about 200 μl, about 75 μL to about 175 μl, about 75 μL to about 150 μl, about 75 μL to about 125 μl, or about 75 μL to about 100 μl.

In some embodiments, the amplifying is in the presence of whole blood proteins, non-target nucleic acids, or both. In some embodiments, the amplifying may be in the presence of from 0.5 μg to 60 μg (e.g., 0.5 μg, 1 μg, 5 μg, 10 μg, 15 μg, 20 μg, 25 μg, 30 μg, 35 μg, 40 μg, 45 μg, 50 μg, 55 μg, or 60 μg) of subject DNA. In some embodiments, the subject DNA is from white blood cells of the subject.

Amplification and Detection of Nucleic Acids from Complex Samples

In some embodiments, methods and systems of the invention can include amplification-based nucleic acid detection assays conducted starting with complex samples (e.g., for diagnostic, forensic, and environmental analyses).

In several embodiments, the methods of the invention involve amplification of one or more nucleic acids. Amplification may be exponential or linear. A target or template nucleic acid may be either DNA or RNA. The sequences amplified in this manner form an “amplified region” or “amplicon.” Primer probes can be readily designed by those skilled in the art to target a specific template nucleic acid sequence. In certain preferred embodiments, resulting amplicons are short to allow for rapid cycling and generation of copies. The size of the amplicon can vary as needed to provide the ability to discriminate target nucleic acids from non-target nucleic acids. For example, amplicons can be less than about 1,000 nucleotides in length. Desirably the amplicons are from 100 to 500 nucleotides in length (e.g., 100 to 200, 150 to 250, 300 to 400, 350 to 450, or 400 to 500 nucleotides in length). In some embodiments, more than one (e.g., 1, 2, 3, 4, 5, 6, 7, 8, 9, 10, or more than 10) target nucleic acids may be amplified in one reaction. In other embodiments, a single target nucleic acid may be amplified in one reaction.

Sample preparation typically involves removing or providing resistance for common PCR inhibitors found in complex samples (e.g., body fluids, tissue homogenates). Common inhibitors are listed in Table 3 (see also, Wilson, Appl. Environ. Microbiol., 63:3741 (1997)). Inhibitors typically act by either prevention of cell lysis, degradation or sequestering a target nucleic acid, and/or inhibition of a polymerase activity. The “facilitators” in Table 3 indicate methodologies or compositions that may be used to reduce or overcome inhibition. The most commonly employed polymerase, Taq, is inhibited by the presence of 0.1% blood in a reaction. Mutant Taq polymerases have been engineered that are resistant to common inhibitors (e.g., hemoglobin and/or humic acid) found in blood (Kermekchiev et al., Nucl. Acid. Res., 37(5): e40, (2009)). Manufacturer recommendations indicate these mutations enable direct amplification from up to 20% blood. Despite resistance afforded by the mutations, accurate real time PCR detection is complicated due to fluorescence quenching observed in the presence of blood sample (Kermekchiev et al., Nucl. Acid. Res., 37:e40 (2009)).

TABLE 3
PCR inhibitors and facilitators for overcoming inhibition.

| Sample or | | | |
|---|---|---|---|
| Specimen | | | |
| Type | Target | Inhibitor | Facilitator |
| feces | Escherichia coli | >10³ bacterial cells | ion-exchange column |
| CSF | Treponema pallidum | Cellular debris causing | nested primers |
| | | nonspecific amplification | |
| whole blood | mammalian | >4 μl of blood/100-ml reaction mix | 1-2% blood per reaction |
| | tissue | (hemoglobin) | |
| feces | Rotatvirus | unknown dilution | cellulose fiber |
| clinical | Cytomegalovirus | unidentified components | glass bead extraction |
| specimens | | | |
| human blood | human genes | DNA binding proteins | thermophilic protease from |
| and tissue | | | Thermus strain rt44A |
| mammalian | Mammalian | thermal cycler variations | formamide |
| tissue | tissue genetics | | |
| mammalian | Mammalian | thermal cycler variations | DMSO, glycerol, PEG, |
| tissue | tissue genetics | | organic solvents |
| clinical | Treponema | unknown factors | Various substrate-specific |
| specimens | pallidum | | physicochemical methods |
| forensic semen | Sperm | Genotyping errors; selective/total | |
| samples | | PCR inhibition by vaginal | |
| | | microorganisms | |
| feces | Salmonella enterica | various body fluids | immunomagnetic |
| | | | separation |
| feces | Various enteric | unknown | size exclusion |
| | viruses | | chromatography, |
| | | | physicochemical extraction |
| clinical | Herpes simplex | endogenous inhibitors, random | repurification, coamplified |
| specimens | virus | effects | positive control |
| feces | Escherichia coli | nonspecific inhibitors, urea, | additional primers and |
| | | hemoglobin, heparin, phenol, | reaction cyclers, booster |
| | | SDS | PCR |
| tissue culture | Cytomegalovirus | glove powder | |
| | HIV | | |
| suspensions, | Mycobacterium leprae | mercury-based fixatives, neutral | reduced fixation times, |
| skin biopsies | | buffered formaline | ethanol fixation |
| clinical | Mycobacterium | unknown inhibitors in pus, tissue | physicochemical extraction |
| specimens | tuberculosis | biopsies, sputum, pleural fluid | |
| mammalian | mammalian | unknown contaminant of reverse | additional DNA |
| tissue | tissue genetics | transcriptase | |
| formalin-fixed | Hepatitus C | ribonucleotide vanadyl complexes | phenol/chloroform |
| paraffin tissue | virus | | extraction |
| nasopharyngeal | Bordetella pertussis | unknown inhibitors | phenol/chloroform |
| aspirates and | | | extraction |
| swabs | | | |
| human | HIV type 1 | detergents | mineral oil |
| mononuclear | | | |
| blood cells | | | |
| bloodstain | human | unidentified heme compound, | BSA |
| | mitochondrial | hemin | |
| | DNA | | |
| blood | various | heparin | alternative polymerases |
| | | | and buffers, chelex, |
| | | | spermine, [Mg2+], glycerol, |
| | | | BSA, heparinase |
| sputa | Mycoplasma pneumoniae | N-acetyl-L-cysteine, dithiothreitol, | |
| | | mucolytic agents | |
| human tissue | HLA-DRB1 | pollen, glove powder, impure | |
| | genotyping | DNA, heparin, hemoglobin | |
| clinical | Mycobacterium | unknown | competitive internal control |
| specimens | tuberculosis | | |
| dental plaque | many | unknown | diatomaceous earth, |
| | | | guanidium isothiocyante, |
| | | | ethanol, acetone |
| ancient | Cytochrome b | unknown | ammonium acetate, |
| mammalian | gene | | ethidium bromide |
| tissues | | | |

Polymerase chain reaction amplification of DNA or cDNA is a tried and trusted methodology; however, as discussed above, polymerases are inhibited by agents contained in crude samples, including but not limited to commonly used anticoagulants and hemoglobin. Recently mutant Taq polymerases have been engineered to harbor resistance to common inhibitors found in blood and soil. Currently available polymerases, e.g., HemoKlenTaq™ (New England BioLabs, Inc., Ipswich, Mass.) as well as OmniTaq™ and OmniKlenTaq™ (DNA Polymerase Technology, Inc., St. Louis, Mo.) are mutant (e.g., N-terminal truncation and/or point mutations) Taq polymerase that render them capable of amplifying DNA in the presence of up to 10%, 20% or 25% whole blood, depending on the product and reaction conditions (See, e.g., Kermekchiev et al. Nucl. Acids Res. 31:6139 (2003); and Kermekchiev et al., Nucl. Acid. Res., 37:e40 (2009); and see U.S. Pat. No. 7,462,475). Additionally, PHUSION® Blood Direct PCR Kits (Finnzymes Oy, Espoo, Finland), include a unique fusion DNA polymerase enzyme engineered to incorporate a double-stranded DNA binding domain, which allows amplification under conditions which are typically inhibitory to conventional polymerases such as Taq or Pfu, and allow for amplification of DNA in the presence of up to about 40% whole blood under certain reaction conditions. See Wang et al., Nuc. Acids Res. 32:1197 (2004); and see U.S. Pat. Nos. 5,352,778 and 5,500,363. Furthermore, Kapa Blood PCR Mixes (Kapa Biosystems, Woburn, Mass.), provide a genetically engineered DNA polymerase enzyme which allows for direct amplification of whole blood at up to about 20% of the reaction volume under certain reaction conditions. Despite these breakthroughs, direct optical detection of generated amplicons is not possible with existing methods since fluorescence, absorbance, and other light based methods yield signals that are quenched by the presence of blood. See Kermekchiev et al., Nucl. Acid. Res., 37:e40 (2009).

The PCR can include any suitable number of cycles, e.g., about 30, about 31, about 32, about 33, about 34, about 35, about 36, about 37, about 38, about 39, about 40, about 41, about 42, about 43, about 44, about 45, about 46, about 47, about 48, about 49, about 50, or more cycles. In some embodiments, the PCR includes about 30-50 cycles, about 35-50 cycles, about 40-50 cycles, about 45-50 cycles, about 46 to 50 cycles, about 30-46 cycles, about 35-46 cycles, about 40-46 cycles, about 45-46 cycles, about 30-45 cycles, about 35-45 cycles, about 40-45 cycles, about 30-40 cycles, or about 35-40 cycles. In some embodiments, the PCR includes 40-46 cycles

A variety of impurities and components of whole blood can be inhibitory to the polymerase and primer annealing. These inhibitors can lead to generation of false positives and low sensitivities. To reduce the generation of false positives and low sensitivities when amplifying and detecting nucleic acids in complex samples, it is desirable to utilize a thermal stable polymerase not inhibited by whole blood samples, for example as described above, and include one or more internal PCR assay controls (see Rosenstraus et al. J. Clin Microbiol. 36:191 (1998) and Hoofar et al., J. Clin. Microbiol. 42:1863 (2004)). For example, the assay can include an internal control nucleic acid that contains primer binding regions identical to those of the target sequence to assure that clinical specimens are successfully amplified and detected. In some embodiments, the target nucleic acid and internal control can be selected such that each has a unique probe binding region that differentiates the internal control from the target nucleic acid. The internal control is, optionally, employed in combination with a processing positive control, a processing negative control, and a reagent control for the safe and accurate determination and identification of an infecting organism in, e.g., a whole blood clinical sample. The internal control can be an inhibition control that is designed to co-amplify with the nucleic acid target being detected. Failure of the internal inhibition control to be amplified is evidence of a reagent failure or process error. Universal primers can be designed such that the target sequence and the internal control sequence are amplified in the same reaction tube. Thus, using this format, if the target DNA is amplified but the internal control is not it is then assumed that the target DNA is present in a proportionally greater amount than the internal control and the positive result is valid as the internal control amplification is unnecessary. If, on the other hand, neither the internal control nor the target is amplified it is then assumed that inhibition of the PCR reaction has occurred and the test for that particular sample is not valid. Exemplary non-limiting internal control nucleic acids that may be used in the methods of the invention include internal control sequences derived from Citrus sinensis or scrambled S. aureus femA nucleic acid sequences.

For example, the Citrus sinensis (orange) internal control (OIC) nucleic acid, which includes the nucleic acid sequence of

(SEQ ID NO: 34)
GGAAATCTAACGAGAGAGCATGCTCCTGCGGCCCCGGAGACGGTGCGCCG
CGGGGTGCGGCGCCTTCTTTCACATGTATCCAAAACGTCTCTCGGCAACG
GATATCTCGGCTCTCGCATCGATGAAGAACGTAGCGAAATGCGATACTTG
GTGTGAATTGCAGAATCCCGTGAACCATCGAGTCTTTGAACGCAAGTTGC
GCCCCAAGCCATTAGGCCGAGGGCACGTCTGCCTGGGTGTCACGCATCG

cloned into plasmid pBR322, may be amplified in the presence of a forward primer comprising the nucleic acid sequence 5′-GGA AAT CTA ACG AGA GAG CAT GCT-3′ (SEQ ID NO: 35) or 5′-GGA AAT CTA ACG AGA GAG CAT GC-3′ (SEQ ID NO: 36) and a reverse primer comprising the nucleic acid sequence 5′-CGA TGC GTG ACA CCC AGG C-3′ (SEQ ID NO: 37) or 5′-GAT GCG TGA CAC CCA GGC-3′ (SEQ ID NO: 38). In some embodiments, an amplicon produced using these primers is detected by hybridization using a 5′ capture probe that includes the oligonucleotide sequence 5′-GAG ACG TTT TGG ATA CAT GTG AAA GAA GGC-3′ (SEQ ID NO: 39) and/or a 3′ capture probe that includes the oligonucleotide sequence 5′-CGA TGG TTC ACG GGA TTC TGC AAT TC-3′ (SEQ ID NO: 40) to detect the presence of the Citrus sinensis internal control nucleic acid in a biological or environmental sample. In some embodiments, the 5′ capture probe and/or the 3′ capture probe is conjugated to a magnetic nanoparticle.

In another example, the randomized S. aureus internal control nucleic acid, which includes the nucleic acid sequence of

(SEQ ID NO: 41)
GAT GCA GCA ACA ACA GAT TCC TTG CTT CTC ATA CAA
TAA CAT GAC AAA CCC CAT TAA TAA AAA CGC GGT CCA
CTT ATC ATA CAG AAT ATC AGA TAG TGG CAA TTA ATT
GTG ACA AAA ATT CGA AAG TTG TGT ACA GTT CTT CAT
TGT TCG AAA AAT TGT TAT GAC AAG ATA CAC CAG GAC
ATA ACG GCT AC

cloned into plasmid pBR322, may be amplified in the presence of a forward primer comprising the nucleic acid sequence 5′-GCA GCA ACA ACA GAT TCC-3′ (SEQ ID NO: 42) and a reverse primer comprising the nucleic acid sequence 5′-GTA GCC GTT ATG TCC TGG TG-3′ (SEQ ID NO: 43). In some embodiments, an amplicon produced using these primers is detected by hybridization using a 5′ capture probe that includes the oligonucleotide sequence 5′-TCG AAC AAT GAA GAA CTG TAC ACA ACT TTC G-3′ (SEQ ID NO: 44) and/or a 3′ capture probe that includes the oligonucleotide sequence 5′-GGT TTG TCA TGT TAT TGT ATG AGA AGC AAG-3′ (SEQ ID NO: 45) to detect the presence of the randomized S. aureus internal control nucleic acid in a biological or environmental sample. In some embodiments, the 5′ capture probe and/or the 3′ capture probe is conjugated to a magnetic nanoparticle.

In other embodiments, an internal control as described in Example 20 of WO 2012/054639 can be used. In some embodiments, the same primer pair used to amplify the Candida target nucleic acid is used to amplify the internal control. For example, in some embodiments, the forward primer includes the oligonucleotide sequence of SEQ ID NO: 1, 2, or 10 and the reverse primer includes the oligonucleotide sequence of SEQ ID NO: 3. For example, in some embodiments, the sequence of the internal control that will be amplified in excess is:

(SEQ ID NO: 24)
5′-GGC ATG CCT GTT TGA GCG TCC TGC ATC ATA CTG AAA
TAG ATC CTT CGA CAA CCT CGG TAC ACT GGG AAC AAG
GCC TCA AAC ATT GAT GCT CGA CTA CAC GTA GGG CAATGC
GTC TTG CTA GAA GCG AAA TCT GTG GCT TGC TAG TGC
AAG CTG GTC GGC GTA TTA TTC CAA CCC GCT GAA CTT
AAG CAT ATC AAT AAG CA-3′

A The annealed complementary sequence is:

(SEQ ID NO: 25)
5′-GCT TAT TGA TAT GCT TAA GTT CAG CGG GTT GGA ATA
ATA CGC CGA CCA GCT TGC ACT AGC AAG CCA CAG ATT
TCG CTT CTA GCA AGA CGC ATT GCC CTA CGT GTA GTC
GAG CAT CAA TGT TTG AGG CCT TGT TCC CAGTGT ACC GAG
GTT GTC GAA GGATCT ATT TCA GTA TGA TGC AGG ACG CTC
AAA CAG GCATGC CA-3′

The above internal control can be detected by hybridization a first nucleic acid probe and a second nucleic acid probe conjugated to one or more populations of magnetic particles. For example, in certain embodiments, the first probe includes the oligonucleotide sequence 5′-GGT TGT CGA AGG ATC TAT TTC AGT ATG ATG CAG-3′ (SEQ ID NO: 26), and the second probe includes the oligonucleotide sequence 5′-TGG AAT AAT ACG CCG ACC AGC TTG CAC TA-3′ (SEQ ID NO: 27).

The assays of the invention can include one or more positive processing controls in which one or more target nucleic acids is included in the assay (e.g., each included with one or more cartridges) at 3× to 5× the limit of detection. The measured T₂ for each of the positive processing controls must be above the pre-determined threshold indicating the presence of the target nucleic acid. The positive processing controls can detect all reagent failures in each step of the process (e.g., lysis, PCR, and T₂ detection), and can be used for quality control of the system. The assays of the invention can include one or more negative processing controls consisting of a solution free of target nucleic acid (e.g., buffer alone). The T₂ measurements for the negative processing control should be below the threshold indicating a negative result while the T₂ measured for the internal control is above the decision threshold indicating an internal control positive result. The purpose of the negative control is to detect carry-over contamination and/or reagent contamination. The assays of the invention can include one or more reagent controls. The reagent control will detect reagent failures in the PCR stage of the reaction (i.e. incomplete transfer of master mix to the PCR tubes). The reagent controls can also detect gross failures in reagent transfer prior to T₂ detection.

In some embodiments, complex biological or environmental samples, which may be a liquid sample (including whole blood, cerebrospinal fluid, urine, synovial fluid, and tissue biopsy homogenates (e.g., skin biopsies) can be directly amplified using about 5%, about 10%, about 20%, about 25%, about 30%, about 25%, about 40%, and about 45% or more complex liquid sample in amplification reactions, and that the resulting amplicons can be directly detected from amplification reaction using magnetic resonance (MR) relaxation measurements upon the addition of conjugated magnetic particles bound to oligonucleotides complementary to the target nucleic acid sequence. Alternatively, the magnetic particles can be added to the sample prior to amplification. Thus, provided are methods for the use of nucleic acid amplification in a complex dirty sample, hybridization of the resulting amplicon to paramagnetic particles, followed by direct detection of hybridized magnetic particle conjugate and target amplicons using magnetic particle based detection systems. In particular embodiments, direct detection of hybridized magnetic particle conjugates and amplicons is via MR relaxation measurements (e.g., T₂, T₁, T₁/T₂ hybrid, T₂*, etc). Further provided are methods which are kinetic, in order to quantify the original nucleic acid copy number within the sample (e.g., sampling and nucleic acid detection at pre-defined cycle numbers, comparison of endogenous internal control nucleic acid, use of exogenous spiked homologous competitive control nucleic acid).

While the exemplary methods described hereinafter relate to amplification using polymerase chain reaction (“PCR”), numerous other methods are known in the art for amplification of nucleic acids (e.g., isothermal methods, rolling circle methods, etc.). Those skilled in the art will understand that these other methods may be used either in place of, or together with, PCR methods. See, e.g., Saiki, “Amplification of Genomic DNA” in PCR Protocols, Innis et al., Eds., Academic Press, San Diego, Calif., pp 13-20 (1990); Wharam et al., Nucleic Acids Res. 29:E54 (2001); Hafner et al., Biotechniques, 30:852 (2001). Further amplification methods suitable for use with the present methods include, for example, polymerase chain reaction (PCR) method, reverse transcription PCR (RT-PCR), ligase chain reaction (LCR), transcription based amplification system (TAS), transcription mediated amplification (TMA), nucleic acid sequence based amplification (NASBA) method, the strand displacement amplification (SDA) method, the loop mediated isothermal amplification (LAMP) method, the isothermal and chimeric primer-initiated amplification of nucleic acid (ICAN) method, and the smart amplification system (SMAP) method. These methods, as well as others are well known in the art and can be adapted for use in conjunction with provided methods of detection of amplified nucleic acid.

The PCR method is a technique for making many copies of a specific template DNA sequence. The PCR process is disclosed in U.S. Pat. Nos. 4,683,195; 4,683,202; and 4,965,188, each of which is incorporated herein by reference. One set of primers complementary to a template DNA are designed, and a region flanked by the primers is amplified by DNA polymerase in a reaction including multiple amplification cycles. Each amplification cycle includes an initial denaturation, and up to 50 cycles of annealing, strand elongation (or extension) and strand separation (denaturation). In each cycle of the reaction, the DNA sequence between the primers is copied. Primers can bind to the copied DNA as well as the original template sequence, so the total number of copies increases exponentially with time. PCR can be performed as according to Whelan, et al, Journal of Clinical Microbiology, 33:556(1995). Various modified PCR methods are available and well known in the art. Various modifications such as the “RT-PCR” method, in which DNA is synthesized from RNA using a reverse transcriptase before performing PCR; and the “TaqMan PCR” method, in which only a specific allele is amplified and detected using a fluorescently labeled TaqMan probe, and Taq DNA polymerase, are known to those skilled in the art. RT-PCR and variations thereof have been described, for example, in U.S. Pat. Nos. 5,804,383; 5,407,800; 5,322,770; and 5,310,652, and references described therein, which are hereby incorporated by reference; and TaqMan PCR and related reagents for use in the method have been described, for example, in U.S. Pat. Nos. 5,210,015; 5,876,930; 5,538,848; 6,030,787; and 6,258,569, which are hereby incorporated by reference.

LCR is a method of DNA amplification similar to PCR, except that it uses four primers instead of two and uses the enzyme ligase to ligate or join two segments of DNA. Amplification can be performed in a thermal cycler (e.g., LCx of Abbott Labs, North Chicago, Ill.). LCR can be performed for example, as according to Moore et al., Journal of Clinical Microbiology 36:1028 (1998). LCR methods and variations have been described, for example, in European Patent Application Publication No. EP0320308, and U.S. Pat. No. 5,427,930, each of which is incorporated herein by reference.

The TAS method is a method for specifically amplifying a target RNA in which a transcript is obtained from a template RNA by a cDNA synthesis step and an RNA transcription step. In the cDNA synthesis step, a sequence recognized by a DNA-dependent RNA polymerase (i.e., a polymerase-binding sequence or PBS) is inserted into the cDNA copy downstream of the target or marker sequence to be amplified using a two-domain oligonucleotide primer. In the second step, an RNA polymerase is used to synthesize multiple copies of RNA from the cDNA template. Amplification using TAS requires only a few cycles because DNA-dependent RNA transcription can result in 10-1000 copies for each copy of cDNA template. TAS can be performed according to Kwoh et al., PNAS 86:1173 (1989). The TAS method has been described, for example, in International Patent Application Publication No. WO 1988/010315, which is incorporated herein by reference.

Transcription mediated amplification (TMA) is a transcription-based isothermal amplification reaction that uses RNA transcription by RNA polymerase and DNA transcription by reverse transcriptase to produce an RNA amplicon from target nucleic acid. TMA methods are advantageous in that they can produce 100 to 1000 copies of amplicon per amplification cycle, as opposed to PCR or LCR methods that produce only 2 copies per cycle. TMA has been described, for example, in U.S. Pat. No. 5,399,491 which is incorporated herein by reference. NASBA is a transcription-based method which for specifically amplifying a target RNA from either an RNA or DNA template. NASBA is a method used for the continuous amplification of nucleic acids in a single mixture at one temperature. A transcript is obtained from a template RNA by a DNA-dependent RNA polymerase using a forward primer having a sequence identical to a target RNA and a reverse primer having a sequence complementary to the target RNA a on the 3′ side and a promoter sequence that recognizes T7 RNA polymerase on the 5′ side. A transcript is further synthesized using the obtained transcript as template. This method can be performed as according to Heim, et al., Nucleic Acids Res., 26:2250 (1998). The NASBA method has been described in U.S. Pat. No. 5,130,238, which is incorporated herein by reference.

The SDA method is an isothermal nucleic acid amplification method in which target DNA is amplified using a DNA strand substituted with a strand synthesized by a strand substitution type DNA polymerase lacking 5′->3′ exonuclease activity by a single stranded nick generated by a restriction enzyme as a template of the next replication. A primer containing a restriction site is annealed to template, and then amplification primers are annealed to 5′ adjacent sequences (forming a nick). Amplification is initiated at a fixed temperature. Newly synthesized DNA strands are nicked by a restriction enzyme and the polymerase amplification begins again, displacing the newly synthesized strands. SDA can be performed according to Walker, et al., PNAS, 89:392 (1992). SDA methods have been described in U.S. Pat. Nos. 5,455,166 and 5,457,027, each of which are incorporated by reference.

The LAMP method is an isothermal amplification method in which a loop is always formed at the 3′ end of a synthesized DNA, primers are annealed within the loop, and specific amplification of the target DNA is performed isothermally. LAMP can be performed according to Nagamine et al., Clinical Chemistry. 47:1742 (2001). LAMP methods have been described in U.S. Pat. Nos. 6,410,278; 6,974,670; and 7,175,985, each of which are incorporated by reference.

The ICAN method is anisothermal amplification method in which specific amplification of a target DNA is performed isothermally by a strand substitution reaction, a template exchange reaction, and a nick introduction reaction, using a chimeric primer including RNA-DNA and DNA polymerase having a strand substitution activity and RNase H. ICAN can be performed according to Mukai et al., J. Biochem. 142: 273(2007). The ICAN method has been described in U.S. Pat. No. 6,951,722, which is incorporated herein by reference.

The SMAP (MITANI) method is a method in which a target nucleic acid is continuously synthesized under isothermal conditions using a primer set including two kinds of primers and DNA or RNA as a template. The first primer included in the primer set includes, in the 3′ end region thereof, a sequence (Ac′) hybridizable with a sequence (A) in the 3′ end region of a target nucleic acid sequence as well as, on the 5′ side of the above-mentioned sequence (Ac′), a sequence (B′) hybridizable with a sequence (Bc) complementary to a sequence (B) existing on the 5′ side of the above-mentioned sequence (A) in the above-mentioned target nucleic acid sequence. The second primer includes, in the 3′ end region thereof, a sequence (Cc′) hybridizable with a sequence (C) in the 3′ end region of a sequence complementary to the above-mentioned target nucleic acid sequence as well as a loopback sequence (D-Dc′) including two nucleic acid sequences hybridizable with each other on an identical strand on the 5′ side of the above-mentioned sequence (Cc′). SMAP can be performed according to Mitani et al., Nat. Methods, 4(3): 257 (2007). SMAP methods have been described in U.S. Patent Application Publication Nos. 2006/0160084, 2007/0190531 and 2009/0042197, each of which is incorporated herein by reference.

The amplification reaction can be designed to produce a specific type of amplified product, such as nucleic acids that are double stranded; single stranded; double stranded with 3′ or 5′ overhangs; or double stranded with chemical ligands on the 5′ and 3′ ends. The amplified PCR product can be detected by: (i) hybridization of the amplified product to magnetic particle bound complementary oligonucleotides, where two different oligonucleotides are used that hybridize to the amplified product such that the nucleic acid serves as an interparticle tether promoting particle agglomeration; (ii) hybridization mediated detection where the DNA of the amplified product must first be denatured; (iii) hybridization mediated detection where the particles hybridize to 5′ and 3′ overhangs of the amplified product; (iv) binding of the particles to the chemical or biochemical ligandson the termini of the amplified product, such as streptavidin functionalized particles binding to biotin functionalized amplified product.

The systems and methods of the invention can be used to perform real time PCR and provide quantitative information about the amount of target nucleic acid present in a sample (see, e.g., FIG. 52 and Example 18 of WO 2012/054639). Methods for conducting quantitative real time PCR are provided in the literature (see for example: RT-PCR Protocols. Methods in Molecular Biology, Vol. 193. Joe O'Connell, ed. Totowa, N.J.: Humana Press, 2002, 378 pp. ISBN 0-89603-875-0.). Example 18 describes use of the methods of the invention for real time PCR analysis of a whole blood sample.

The systems and methods of the invention can be used to perform real time PCR directly in opaque samples, such as whole blood, using magnetic nanoparticles modified with capture probes and magnetic separation. Using real-time PCR allows for the quantification of a target nucleic acid without opening the reaction tube after the PCR reaction has commenced.

In one approach, biotin or avidin labeled primers can be used to perform real-time PCR. These labels would have corresponding binding moieties on the magnetic particles that could have very fast binding times. This allows for a double stranded product to be generated and allows for much faster particle binding times, decreasing the overall turnaround time. The binding chemistry would be reversible, preventing the primers from remaining particle bound. In order to reverse the binding, the sample can be heated or the pH adjusted.

In another approach, the real-time PCR can be accomplished through the generation of duplex DNA with overhangs that can hybridize to the superparamagnetic particles. Additionally, LNA and/or fluorinated capture probes may speed up the hybridization times.

In still another approach, the particles are designed to have a hairpin that buries the capture probe binding site to the amplicon. Heating the particles to a higher melt temperature would expose the binding site of the hairpin of the capture probes on the particles to allow binding to the target.

In another approach, a probe that hybridizes to an amplicon is tethering two (or more) particles. The reaction would be conducted in the presence of a polymerase with 5′ exonuclease activity, resulting in the cleavage of the inter-particle tether and a subsequent change in T₂. The polymerase is selected to have exonuclease activity and compatibility with the matrix of choice (e.g. blood). In this approach, smaller particles (e.g., 30 nm CLIO) can be used to reduce steric hindrance of the hybridization to target or subsequent enzymatic digestion during polymerization (see, e.g., Heid et al Genome Research 1996 6: 986-994).

In another approach, two particle populations can be synthesized to bear complementary capture probes. In the absence of amplicon, the capture probes hybridize promoting particle clustering. Upon generation of amplicon, the amplicon can compete, hybridize, and displace the capture probes leading to particle declustering. The method can be conducted in the presence or absence of nanoparticles. The particles free in solution will cluster and decluster due to the thermocycling (because, e.g., the Tm can be below 95° C.). The Tm of the amplicon binding to one of the particle-immobilized capture probes can be designed such that that binding interaction is more favorable than the particle-to-particle binding interaction (by, e.g., engineering point mutations within the capture probes to thermodynamically destabilize the duplexes). In this embodiment, the particle concentration can be kept at, e.g., low or high levels.

Previous work showed that in some cases the presence of particles in the PCR reaction could inhibit PCR. For these inhibitory particles, it is envisioned that the particles could be pulled to the side of the tube (or other location within the container) to keep them out of solution during the PCR reaction. Methods can be used to release the particles back into suspension to allow them to hybridize to the PCR product and then pull them back out of solution. Other previous work has shown that specific formulations of particles are not inhibitory to the PCR reaction and can remain in solution during amplification.

In certain embodiments, the invention features the use of enzymes compatible with whole blood, including but not limited to NEB HemoKlenTaq™, DNAP OmniKlenTaq™, Kapa Biosystems whole blood enzyme, and Thermo-Fisher Finnzymes Phusion® enzyme.

The invention also features quantitative asymmetric PCR. In any of the real-time PCR methods of the invention, the method can involve the following steps:
1. aliquoting whole blood into a prepared PCR mastermix containing superparamagnetic particles;
2. prior to the first PCR cycle, closing the tube until PCR cycling is completed;
3. loading the tube onto thermal cycler;
4. running “n” cycles of standard PCR thermal cycling;
5. conducting a T2 detection (the exact time duration and steps for this vary depending on the biochemical and particle design approach described below); and
6. repeating steps 4 and 5 until enough T2 readings have been taken for an accurate quantification of initial target concentration.

The above methods can be used with any of the following categories of detection of aggregation or disaggregation described herein, including those described in Table 4.

TABLE 4
Categories of Detection of Aggregation or Disaggregation

| Name | Description |
|---|---|
| Clustering-based detection and | Particles >100 nm or magnetic-separation compatible. |
| magnetic separation | Particles removed from solution during PCR |
| | T₂ goes up with amplicon generation |
| | Agitation during step 5 |
| Clustering-based detection with | Particles >100 nm |
| particles >100 nm | Particles do not inhibit PCR |
| | T₂ goes up with amplicon generation |
| | Agitation during step 5 |
| De-clustering-based detection | Particles >100 nm |
| and magnetic separation | Particles on the side of the tube during PCR |
| | T₂ goes down with amplicon generation |
| | Agitation during step 5 |
| De-clustering-based detection | Particles >100 nm |
| with particles >100 nm | Particles do not inhibit PCR |
| | T₂ goes down with amplicon generation |
| | Agitation during step 5 |
| Clustering-based detection with | Particles <100 nm (e.g., 30 nm particles) |
| particles <100 nm | T₂ goes down with amplicon appearance (at least for initial |
| | cycles, T₂ may subsequently increase as cluster size |
| | increases) |
| | Has potential for much more rapid hybridization times |
| | No agitation required to keep particles suspended |
| | Particle concentration in nM range |
| De-clustering-based detection | Particles <100 nm (e.g., 30 nm particles) |
| with particles <100 nm | T2 goes up with amplicon appearance |
| | T2 could decrease as the cluster size increase above |
| | 100 nm |
| | No agitation required to keep particles suspended |
| | Has potential for most rapid detection times |
| | Particle concentration in nM range |

In any of the methods described herein, the amplified target nucleic acid(s) can be detected by any suitable method, including, without limitation, T2MR-based detection, sequencing (e.g., Sanger sequencing or a high-throughput sequencing approach (e.g., ILLUMINA sequencing), optical, fluorescent, mass, density, magnetic, chromatographic, and/or electrochemical measurement of the amplified liquid sample.

Contamination Control

One potential problem in the use of PCR as an analytical tool is the risk of having new reactions contaminated with old, amplified products. Potential sources of contamination include a) large numbers of target organisms in clinical specimens that may result in cross-contamination, b) plasmid clones derived from organisms that have been previously analyzed and that may be present in larger numbers in the laboratory environment, and c) repeated amplification of the same target sequence leading to accumulation of amplification products in the laboratory environment. A common source of the accumulation of the PCR amplicon is aerosolization of the product. Typically, if uncontrolled aerosolization occurs, the amplicon will contaminate laboratory reagents, equipment, and ventilation systems. When this happens, all reactions will be positive, and it is not possible to distinguish between amplified products from the contamination or a true, positive sample. In addition to taking precautions to avoid or control this carry-over of old products, preferred embodiments include a blank reference reaction in every PCR experiment to check for carry-over. For example, carry-over contamination will be visible on the agarose gel as faint bands or fluorescent signal when TaqMan probes, MolBeacons, or intercalating dyes, among others, are employed as detection mechanisms. Furthermore, it is preferred to include a positive sample. As an example, in some embodiments, contamination control is performed using any of the approaches and methods described in WO 2012/054639.

Typically, the instrumentation and processing areas for samples that undergo amplification are split into pre- and post-amplification zones. This minimizes the chances of contamination of samples with amplicon prior to amplification. For example, the T2Dx® instrument design is such that the pre- and post-amplification instrumentation and processing areas are integrated into a single instrument. This is made possible as described in the sections below.

Systems

The invention features systems for carrying out the methods of the invention, which may include one or more NMR units, MAA units, cartridge units, and agitation units, as described in WO 2012/054639.

Such systems may further include other components for carrying out an automated assay of the invention, such as a thermocycling unit for the amplification of oligonucleotides; a centrifuge, a robotic arm for delivery an liquid sample from unit to unit within the system; one or more incubation units; a fluid transfer unit (i.e., pipetting device) for combining assay reagents and a biological or environmental sample to form the liquid sample; a computer with a programmable processor for storing data, processing data, and for controlling the activation and deactivation of the various units according to a one or more preset protocols; and a cartridge insertion system for delivering pre-filled cartridges to the system, optionally with instructions to the computer identifying the reagents and protocol to be used in conjunction with the cartridge. FIG. 42 of WO 2012/054639 depicts an exemplary system of the invention.

The systems of the invention can provide an effective means for high throughput and real-time detection of analytes present in a bodily fluid from a subject. The detection methods may be used in a wide variety of circumstances including, without limitation, identification and/or quantification of analytes that are associated with specific biological processes, physiological conditions, disorders or stages of disorders. As such, the systems have a broad spectrum of utility in, for example, disease diagnosis, parental and forensic identification, disease onset and recurrence, individual response to treatment versus population bases, and monitoring of therapy. The devices and systems can provide a flexible system for personalized medicine. The system of the invention can be changed or interchanged along with a protocol or instructions to a programmable processor of the system to perform a wide variety of assays as described herein. The systems of the invention offer many advantages of a laboratory setting contained in a desk-top or smaller size automated instrument.

The systems of the invention can be used to simultaneously assay analytes that are present in the same liquid sample over a wide concentration range, and can be used to monitor the rate of change of an analyte concentration and/or or concentration of PD or PK markers over a period of time in a single subject, or used for performing trend analysis on the concentration, or markers of PD, or PK, whether they are concentrations of drugs or their metabolites. Thus, the data generated with the use of the subject fluidic devices and systems can be utilized for performing a trend analysis on the concentration of an analyte in a subject.

For example, a subject (e.g., a patient having or suspected of having a disease caused by or associated with a Candida species (e.g., Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, or C. tropicalis), such as Candidiasis, Candidemia, or sepsis) may be provided with a plurality of cartridge units to be used for detecting a variety of analytes, such as analytes sampled from different tissues, and at predetermined times. A subject may, for example, use different cartridge units on different days of the week. In some embodiments the software on the system is designed to recognize an identifier on the cartridge instructing the system computer to run a particular protocol for running the assay and/or processing the data. The protocols on the system can be updated through an external interface, such as an USB drive or an Ethernet connection, or in some embodiments the entire protocol can be recorded in the barcode attached to the cartridge. The protocol can be optimized as needed by prompting the user for various inputs (i.e., for changing the dilution of the sample, the amount of reagent provided to the liquid sample, altering an incubation time or MAA time, or altering the NMR relaxation collection parameters).

A multiplexed assay can be performed using a variety of system designs. For example, a multiplexed assay can performed using any of the following configurations:

(i) a spatially-based detection array can be used to direct magnetic particles to a particular region of a tube (i.e., without aggregation) and immobilize the particles in different locations according to the particular analyte being detected. The immobilized particles are detected by monitoring their local effect on the relaxation effect at the site of immobilization. The particles can be spatially separated by gravimetric separation in flow (i.e., larger particles settling faster along with a slow flow perpendicular to gravity to provide spatial separation based on particle size with different magnetic particle size populations being labeled with different targets). Alternatively, of capture probes can be used to locate magnetic particles in a particular region of a tube (i.e., without aggregation) and immobilize the particles in different locations (i.e., on a functionalized surface, foam, or gel). Optionally, the array is flow through system with multiple coils and magnets, each coil being a separate detector that has the appropriate particles immobilized within it, and the presence of the analyte detected with signal changes arising from clustering in the presence of the analyte. Optionally, once the particles are spatially separated, each individual analyte in the multiplexed assay can be detected by sliding a coil across the sample to read out the now spatially separated particles.

(ii) A microfluidic tube where the sample is physically split amongst many branches and a separate signal is detected in each branch, each branch configured for detection of a separate analyte in the multiplexed assay.

(iii) An array of 96 wells (or less or more) where each well has its own coil and magnet, and each well is configured for detection of a separate analyte in the multiplexed assay.

(iv) A sipper or flow through device with multiple independently addressable coils inside one magnet or inside multiple mini magnets that can be used for sequential readings, each reading being a separate reaction for detection of a separate analyte in the multiplexed assay.

(v) A sipper or flow through device with multiple independently addressable wells on a plate inside one magnet or inside multiple mini magnets that can be used for sequential readings using a single sided coil that can be traversed along the plate, each reading being a separate reaction for detection of a separate analyte in the multiplexed assay.

(vi) A tube containing two compartments read simultaneously, resulting in one relaxation curve which is then fit using bi-exponential fitting to produce the separate readings for the multiplexed array.

(vii) A microfluidics system where each droplet of liquid is moved around individually, to produce readings for the multiplexed array.

(viii) Sequential measurements using magnetic separation and resuspension requires novel binding probes or the ability to turn them on and off. This method would be used for nucleic acid analytes in which turn on/off mechanism is based mostly on melting temperature (at higher temperatures hairpin loops relax, denaturation of double strand binding), and hybridization will occur at different temperatures.

(ix) Individual capillaries, each equipped with dried particles within them, allow for small volume rapid multiplexing of one small aliquot. The dried particles are spatially separated, and this spatial separation permits the MR Reader to read each capillary tube independently.

(x) Binding moieties conjugated to nanoparticles are placed in a gel or other viscous material forming a region and analyte specific viscous solution. The gel or viscous solution enhances spatial separation of more than one analyte in the starting sample because after the sample is allowed to interact with the gel, the target analyte can readily diffuse through the gel and specifically bind to a conjugated moiety on the gel or viscous solution held nanoparticle. The clustering or aggregation of the specific analyte, optionally enhanced via one of the described magnetic assisted agglomeration methods, and detection of analyte specific clusters can be performed by using a specific location NMR reader. In this way a spatial array of nanoparticles, and can be designed, for example, as a 2d array.

(xi) Magnetic particles can be spotted and dried into multiple locations in a tube and then each location measured separately. For example, one type of particle can be bound to a surface and a second particle suspended in solution, both of which hybridize to the analyte to be detected. Clusters can be formed at the surface where hybridization reactions occur, each surface being separately detectable.

(xii) A spotted array of nucleic acids can be created within a sample tube, each configured to hybridize to a first portion of an array of target nucleic acids. Magnetic particles can be designed with probes to hybridize to a second portion of the target nucleic acid. Each location can be measured separately. Alternatively, any generic beacon or detection method could be used to produce output from the nucleic acid array.

(xiii) An array of magnetic particles for detecting an array of targets can be included in a single sample, each configured (e.g., by size, or relaxation properties) to provide a distinct NMR relaxation signature with aggregate formation. For example, each of the particles can be selected to produce distinct T₂ relaxation times (e.g., one set of particles covers 10-200 ms, a second set from 250-500 ms, a third set from 550-1100 ms, and so on). Each can be measured as a separate band of relaxation rates.

(xiv) For detection of analytes of various size or magnetic particles, or aggregates of various size, a single sample with multiple analytes and magnetic particles can undergo separation in the presence of a magnetic or electric field (i.e., electrophoretic separation of magnetic particles coated with analytes), the separate magnetic particles and/or aggregates reaching the site of a detector at different times, accordingly.

(xv) The detection tube could be separated into two (or more) chambers that each contain a different nanoparticle for detection. The tube could be read using the reader and through fitting a multiple exponential curve such as A*exp(T2_1)+B*exp(T2_2), the response of each analyte could be determined by looking at the relative size of the constants A and B and T2_1 and T2_2.

(xvi) Gradient magnetic fields can be shimmed to form narrow fields. Shim pulses or other RF based Shimming within a specific field can be performed to pulse and receive signals within a specific region. In this way one could envision a stratification of the RF pulse within a shim and specific resonance signals could be received from the specific shim. While this method relies on shimming the gradient magnetic field, multiplexing would include then, to rely on one of the other methods described to get different nanoparticles and the clusters to reside in these different shims. Thus there would be two dimensions, one provided by magnetic field shims and a second dimension provided by varying nanoparticle binding to more than one analyte. Nanoparticles having two distinct NMR relaxation signals upon clustering with an analyte may be employed in a multiplexed assay. In this method, the observation that small particles (30-200 nm) cause a decrease in T₂ with clustering whereas large particles (>800 nm) cause an increase with clustering. The reaction assay is designed as a competitive reaction, so that with the addition of the target it changes the equilibrium relaxation signal. For example, if the T₂ relaxation time is shorter, clusters forming of analyte with small particles are forming. If on the other hand, the T₂ relaxation becomes longer, clusters of analyte with larger particles are forming. It's probably useful to change the density/viscosity of the solution with additives such as trehalose or glucose or glycerol to make sure the big particles stay in solution. One nanoparticle having binding moieties to a specific analyte for whose T₂ signal is decreased on clustering may be combined with a second nanoparticle having a second binding moiety to a second analyte for whose T₂ signal is increased on clustering. In the case for which the sample is suspected to have both analytes and the clustering reaction may cancel each other out (the increased clustering cancels the decreased clustering), one could envision an ordering of the analysis, i.e. addition of competitive binding agents to detect a competitive binding and thus T₂ signal that would be related to the presence/absence of the analyte of interest in the sample. Alternatively, if the increased clustering cancels the decreased clustering in this multiplexing format, one could envision use of different relaxation pulse sequences or relaxation determinants to identify the presence/absence or concentration of analyte in the sample.

(xvii) Precipitation measurement of particles. In this method, multiple types of particles designed to capture different target sequences of nucleic acid are designed So that the particle size is small enough that the particles bound with analyte remain suspended in solution. Sequential addition of an “initiator” sequence that is complementary to a nucleic acid sequence conjugated to a second set of particles (a larger particle, not necessarily having magnetic properties) and contains a complementary sequence to the captured target DNA sequence. After hybridization, clusters will form if the target DNA sequence is present, e.g. the magnetic nanoparticle conjugated with probe anneals to one specific sequence on the target analyte and the other particle binds to another sequence on the target nucleic acid sequence. These clusters will be big enough to precipitate (this step may require a centrifugation step). In the same reaction, and simultaneously, one could design an additional magnetic particle, second particle set to anneal with a second nucleic acid sequence for which formation of the magnetic nanoparticle-analyte-second particle clusters do not precipitate. In this way sequential addition of particles can result in differential signaling.

(xviii) One possible different detection technique includes phase separated signals, which would stem from differing RF coil pulse sequences that are optimized for the conjugated nanoparticle-analyte interaction. Optimally, this could be achieved with multiple coils in an array that would optimize the ability of the different RF pulses and relaxation signal detection to be mapped and differentiated to ascertain the presence/absence of more than one analyte. Multiplexing may also employ the unique characteristic of the nanoparticle-analyte clustering reaction and subsequent detection of water solvent in the sample, the ability of the clusters to form various “pockets” and these coordinated clusters to have varying porosity. For example, linkers having varying length or conformational structures can be employed to conjugate the binding moiety to the magnetic nanoparticle. In this way, more than one type of cluster formed in the presence of an analyte could be designed having the ability of differing solvent water flow, and thus relaxation signal differences, through the aggregated nanoparticle-analyte-nanoparticle formation. In this way, two or more linker/binding moiety designs would then allow for detection of more than one analyte in the same sample.

(xix) The methods of the invention can include a fluorinated oil/aqueous mixture for capturing particles in an emulsion. In this design one hydrophobic capture particle set and an aqueous capture set are used, the hydrophobic capture particle set is designed to bind and aggregate more readily in an hydrophobic environment, whereas the aqueous capture particle set is designed to bind and aggregate in an aqueous environment. Introduction of an analyte containing sample having specific analytes that will bind to either the hydrophobic or aqueous particle, and subsequent mixing in the detection tube having both hydrophobic and aqueous solvents, binding and clustering would then result in a physical separation of analytes to either the aqueous or hydrophobic phase. The relaxation signal could be detected in either solution phase. In the event that the analytes and nanoparticles designed in this manner are physically found in an emulsion created by the mixing of the hydrophobic/aqueous phases, relaxation curves would be distinguishable in the emulsion phase. The detection tube may have a capsular design to enhance the ability to move the capsules through an MR detector to read out the signal. Further, additional use of a fluorescent tag to read out probe identity may be employed, i.e. in the case of two different analytes in the same aqueous or hydrophobic phase, the addition of a fluorescent tag can assist determination of the identity of the analyte. This method is amenable in samples for which limited isolation or purification of the target analyte away from the other material in the sample because the described resonance signals are independent of sample quality. Further, the addition of the fluorescent tag can be added in much higher concentrations that usually added in typical fluorescent studies because these tags will never interfere with the relaxation measurements. In this method, oligonucleotide capture probes that are conjugated to the magnetic nanoparticles are designed so that specific restriction endonuclease sites are located within the annealed section. After hybridization with the sample forming nanoparticle-analyte clusters, a relaxation measurement then provides a base signal. Introduction of a specific restriction endonuclease to the detection tube and incubation will result in a specific reduction of the nanoparticle/analyte cluster after restriction digestion has occurred. After a subsequent relaxation measurement, the pattern of signal and restriction enzyme digestion, one can deduce the target.

(xx) In a combined method, a magnetic nanoparticle is conjugated with two separate and distinct binding moieties, i.e. an oligonucleotide and an antibody. This nanoparticle when incubated with a sample having both types of analytes in the sample will form nanoparticle-analyte complexes, and a baseline T₂ relaxation signal will be detectable. Subsequent addition of a known concentration of one of the analytes can be added to reduce the clustering formed by that specific analyte from the sample. After known analyte addition a subsequent T₂ relaxation signal is detected and the presence/absence of the sample analyte can be surmised. Further, a second analyte can be added to compete with the analyte in the sample to form clusters. Again, after a subsequent T₂ relaxation signal detection the presence/absence of the second sample analyte can be surmised. This can be repeated.

Broadly a multiplexed assay employing the methods of this invention can be designed so that the use of one non-superparamagnetic nanoparticle to generate clusters with analyte from a sample, will reduce the overall Fe²⁺ in assay detection vessel and will extend the dynamic range so that multiple reactions can be measured in the same detection vessel.

Multiplexing nucleic acid detection can make use of differing hybridization qualities of the conjugated magnetic nanoparticle and the target nucleic acid analyte. For example, capture probes conjugated to magnetic nanoparticles can be designed so that annealing the magnetic nanoparticle to the target nucleic acid sequence is different for more than one nucleic acid target sequence. Factors for the design of these different probe-target sequences include G-C content (time to form hybrids), varying salt concentration, hybridization temperatures, and/or combinations of these factors. This method then would entail allowing various nucleic acid conjugated magnetic nanoparticles to interact with a sample suspected of having more than one target nucleic acid analyte. Relaxation times detected after various treatments, i.e. heating, addition of salt, hybridization timing, would allow for the ability to surmise which suspected nucleic acid sequence is present or absent in the sample.

Use complimentary amplicons to block one reaction and allow serial hybridizations. In this method, universal amplification primers are used to amplify more than one specific nucleic acid sequence in the starting sample, forming an amplicon pool. Specific oligonucleotide conjugated to magnetic nanoparticles are added to the sample and a relaxation measurement is taken. The sample is then exposed to a temperature to melt the oligonucleotide-analyte interaction and addition of an oligonucleotide that is not attached to a magnetic nanoparticle is added to compete away any analyte binding to the magnetic nanoparticle. A second magnetic nanoparticle having a second oligonucleotide conjugated to it is then added to form clusters with a second specific target nucleic acid analyte. Alternatively, the method could have a step prior to the addition of the second magnetic nanoparticle that would effectively sequester the first magnetic nanoparticle from the reaction vessel, i.e. exposing the reaction vessel to a magnetic field to move the particles to an area that would not be available to the second, or subsequent reaction.

Each of the multiplexing methods above can employ a step of freezing the sample to slow diffusion and clustering time and thus alter the measurement of the relaxation time. Slowing the diffusion and clustering of the method may enhance the ability to separate and detect more than one relaxation time. Each of the multiplexing methods above can make use of sequential addition of conjugated nanoparticles followed by relaxation detection after each addition. After each sequential addition, the subsequent relaxation baseline becomes the new baseline from the last addition and can be used to assist in correlating the relaxation time with presence/absence of the analyte or analyte concentration in the sample.

In some embodiments, the method of multiplexing may involve hidden capture probes. In this method of multiplexing, oligonucleotides conjugated to the magnetic nanoparticles are designed so that secondary structure or a complementary probe on the surface of the particle hides or covers the sequence for hybridization initially in the reaction vessel. These hidden hybridization sequences are then exposed or revealed in the sample vessel spatially or temporally during the assay. For example, as mentioned above, hybridization can be affected by salt, temperature and time to hybridize. Thus, in one form of this method, secondary or complementary structures on the oligonucleotide probe conjugated to the magnetic nanoparticle can be reduced or relaxed to then expose or reveal the sequence to hybridize to the target nucleic acid sample. Further, secondary structures could be reduced or relaxed using a chemical compound, e.g., dimethyl sulfoxide (DMSO). Another method to selectively reveal or expose a sequence for hybridization of the oligonucleotide conjugated nanoparticle with the target analyte is to design stem-loop structures having a site for a restriction endonuclease; subsequent digestion with a restriction endonuclease would relax the stem-loop structure and allow for hybridization to occur. Alternatively, a chemical cut of the stem-loop structure, releasing one end could make the sequence free to then hybridize to the target nucleic acid sequence.

Where the multiplexed array is configured to detect a target nucleic acid, the assay can include a multiplexed PCR to generate different amplicons and then serially detect the different reactions.

The multiplexed assay optionally includes a logical array in which the targets are set up by binary search to reduce the number of assays required (e.g., gram positive or negative leads to different species based tests that only would be conducted for one group or the other).

The systems of the invention can run a variety of assays, regardless of the analyte being detected from a bodily fluid sample. A protocol dependent on the identity of the cartridge unit being used can be stored on the system computer. In some embodiments, the cartridge unit has an identifier (ID) that is detected or read by the system computer, or a bar code (1D or 2D) on a card that then supplies assay specific or patient or subject specific information needed to be tracked or accessed with the analysis information (e.g., calibration curves, protocols, previous analyte concentrations or levels). Where desired, the cartridge unit identifier is used to select a protocol stored on the system computer, or to identify the location of various assay reagents in the cartridge unit. The protocol to be run on the system may include instructions to the controller of the system to perform the protocol, including but not limited to a particular assay to be run and a detection method to be performed. Once the assay is performed by the system, data indicative of an analyte in the biological or environmental sample is generated and communicated to a communications assembly, where it can either be transmitted to the external device for processing, including without limitation, calculation of the analyte concentration in the sample, or processed by the system computer and the result presented on a display readout.

For example, the identifier may be a bar code identifier with a series of black and white lines, which can be read by a bar code reader (or another type of detector) upon insertion of the cartridge unit. Other identifiers could be used, such as a series of alphanumerical values, colors, raised bumps, RFID, or any other identifier which can be located on a cartridge unit and be detected or read by the system computer. The detector may also be an LED that emits light which can interact with an identifier which reflects light and is measured by the system computer to determine the identity of a particular cartridge unit. In some embodiments, the system includes a storage or memory device with the cartridge unit or the detector for transmitting information to the system computer.

Thus, the systems of the invention can include an operating program to carry out different assays, and cartridges encoded to: (i) report to the operating program which pre-programmed assay was being employed; (ii) report to the operating program the configuration of the cartridges; (iii) inform the operating system the order of steps for carrying out the assay; (iv) inform the system which pre-programmed routine to employ; (v) prompt input from the user with respect to certain assay variables; (vi) record a patient identification number (the patient identification number can also be included on the vacutainer holding the blood sample); (vii) record certain cartridge information (e.g., lot number, calibration data, assays on the cartridge, analytic data range, expiration date, storage requirements, acceptable sample specifics); or (viii) report to the operating program assay upgrades or revisions (i.e., so that newer versions of the assay would occur on cartridge upgrades only and not to the larger, more costly system).

The systems of the invention can include one or more fluid transfer units configured to adhere to a robotic arm (see, e.g., FIGS. 43A-43C of WO 2012/054639). The fluid transfer unit can be a pipette, such as an air-displacement, liquid backed, or syringe pipette. For example, a fluid transfer unit can further include a motor in communication with a programmable processor of the system computer and the motor can move the plurality of heads based on a protocol from the programmable processor. Thus, the programmable processor of a system can include instructions or commands and can operate a fluid transfer unit according to the instructions to transfer liquid samples by either withdrawing (for drawing liquid in) or extending (for expelling liquid) a piston into a closed air space. Both the volume of air moved and the speed of movement can be precisely controlled, for example, by the programmable processor. Mixing of samples (or reagents) with diluents (or other reagents) can be achieved by aspirating components to be mixed into a common tube and then repeatedly aspirating a significant fraction of the combined liquid volume up and down into a tip. Dissolution of reagents dried into a tube can be done is similar fashion.

A system can include one or more incubation units for heating the liquid sample and/or for control of the assay temperature. Heat can be used in the incubation step of an assay reaction to promote the reaction and shorten the duration necessary for the incubation step. A system can include a heating block configured to receive a liquid sample for a predetermined time at a predetermined temperature. The heating block can be configured to receive a plurality of samples.

The system temperature can be carefully regulated. For example, the system includes a casing kept at a predetermined temperature (i.e., 37° C.) using stirred temperature controlled air. Waste heat from each of the units will exceed what can be passively dissipated by simple enclosure by conduction and convection to air. To eliminate waste heat, the system can include two compartments separated by an insulated floor. The upper compartment includes those portions of the components needed for the manipulation and measurement of the liquid samples, while the lower compartment includes the heat generating elements of the individual units (e.g., the motor for the centrifuge, the motors for the agitation units, the electronics for each of the separate units, and the heating blocks for the incubation units). The lower floor is then vented and forced air cooling is used to carry heat away from the system. See, e.g., FIGS. 44A and 44B of WO 2012/054639.

The MR unit may require more closely controlled temperature (e.g., ±0.1° C.), and so may optionally include a separate casing into which air heated at a predetermined temperature is blown. The casing can include an opening through which the liquid sample is inserted and removed, and out of which the heated air is allowed to escape. See, e.g., FIGS. 45A and 45B of WO 2012/054639. Other temperature control approaches may also be utilized.

Cartridge Units

The invention features methods and systems that may involve one or more cartridge units to provide a convenient method for placing all of the assay reagents and consumables onto the system. For example, the system may be customized to perform a specific function, or adapted to perform more than one function, e.g., via changeable cartridge units containing arrays of micro wells with customized magnetic particles contained therein. The system can include a replaceable and/or interchangeable cartridge containing an array of wells pre-loaded with magnetic particles, and designed for detection and/or concentration measurement of a particular analyte. Alternatively, the system may be usable with different cartridges, each designed for detection and/or concentration measurements of different analytes, or configured with separate cartridge modules for reagent and detection for a given assay. The cartridge may be sized to facilitate insertion into and ejection from a housing for the preparation of a liquid sample which is transferred to other units in the system (e.g., a magnetic assisted agglomeration unit, or an NMR unit). The cartridge unit itself could potentially interface directly with manipulation stations as well as with the MR reader(s). The cartridge unit can be a modular cartridge having an inlet module that can be sterilized independent of the reagent module.

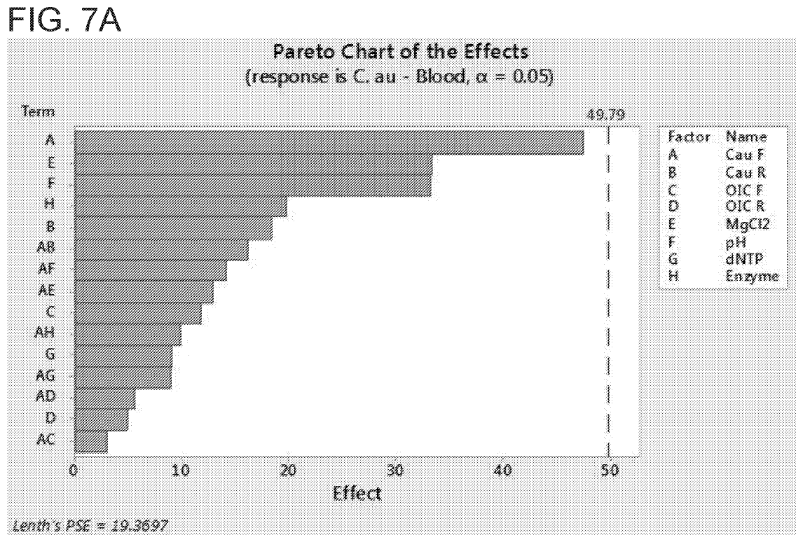

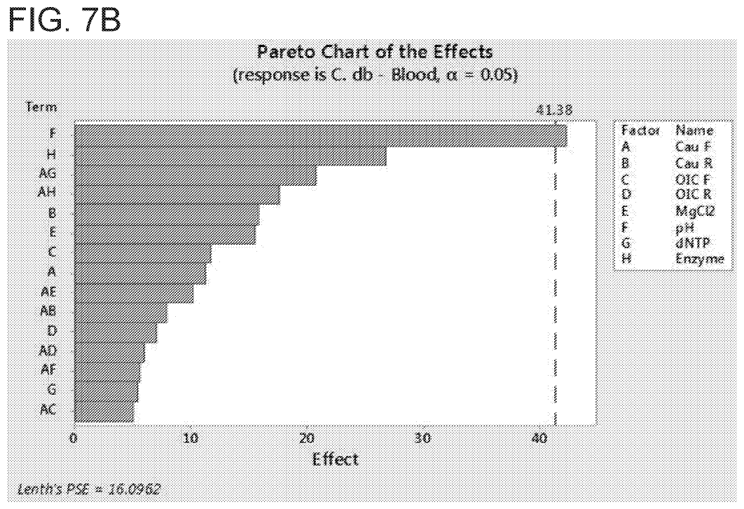

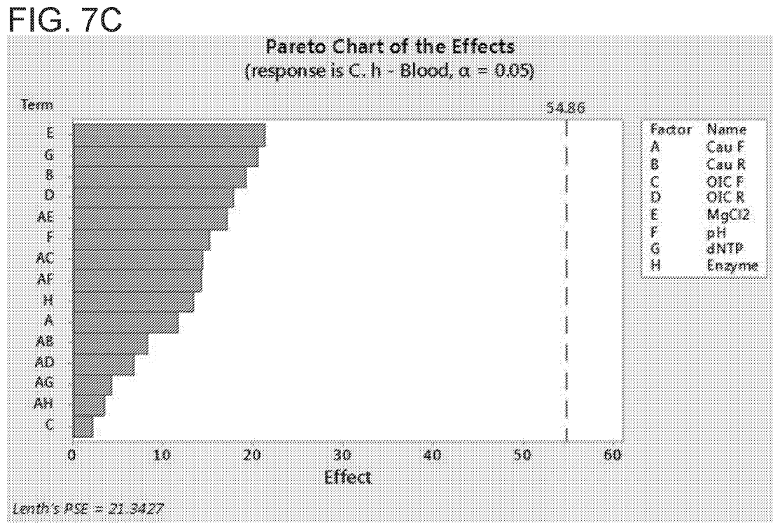

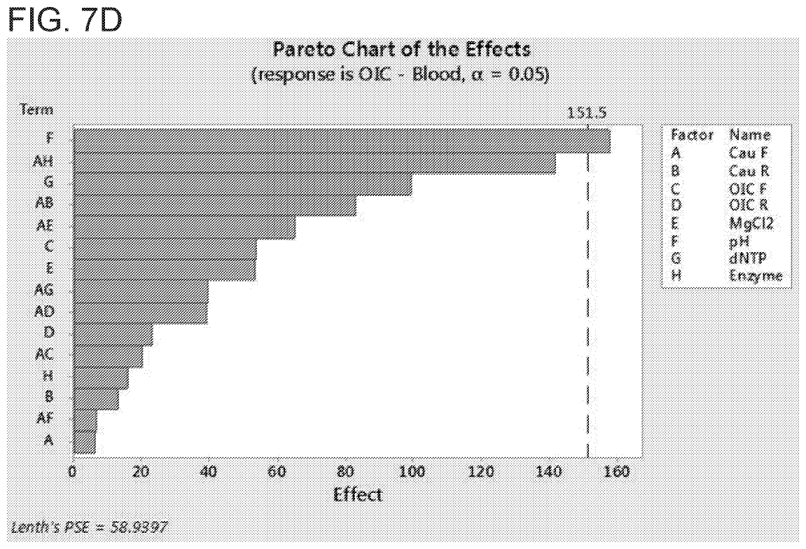

For handling biological or environmental samples, such as blood samples, there are numerous competing requirements for the cartridge design, including the need for sterility for the inlet module to prevent cross contamination and false positive test results, and the need to include reagents in the package which cannot be easily sterilized using standard terminal sterilization techniques like irradiation. An inlet module for sample aliquoting can be designed to interface with uncapped vacutainer tubes, and to aliquot two a sample volume that can be used to perform, for example, an assay to detect a Candida species (see FIGS. 7D-7F of WO 2012/054639). The vacutainer permits a partial or full fill. The inlet module has two hard plastic parts, that get ultrasonically welded together and foil sealed to form a network of channels to allow a flow path to form into the first well overflow to the second sample well. A soft vacutainer seal part is used to for a seal with the vacutainer, and includes a port for sample flow, and a venting port. To overcome the flow resistance once the vacutainer is loaded and inverted, some hydrostatic pressure is needed. Every time sample is removed from a sample well, the well will get replenished by flow from the vacutainer.

A modular cartridge can provide a simple means for cross contamination control during certain assays, including but not limited to distribution of amplification (e.g., PCR products) into multiple detection aliquots. In addition, a modular cartridge can be compatible with automated fluid dispensing, and provides a way to hold reagents at very small volumes for long periods of time (in excess of a year). Finally, pre-dispensing these reagents allows concentration and volumetric accuracy to be set by the manufacturing process and provides for a point of care use instrument that is more convenient as it can require much less precise pipetting.

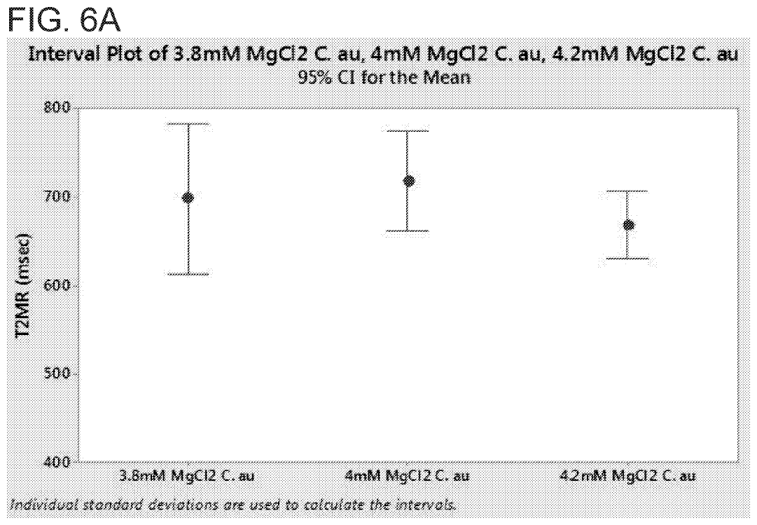

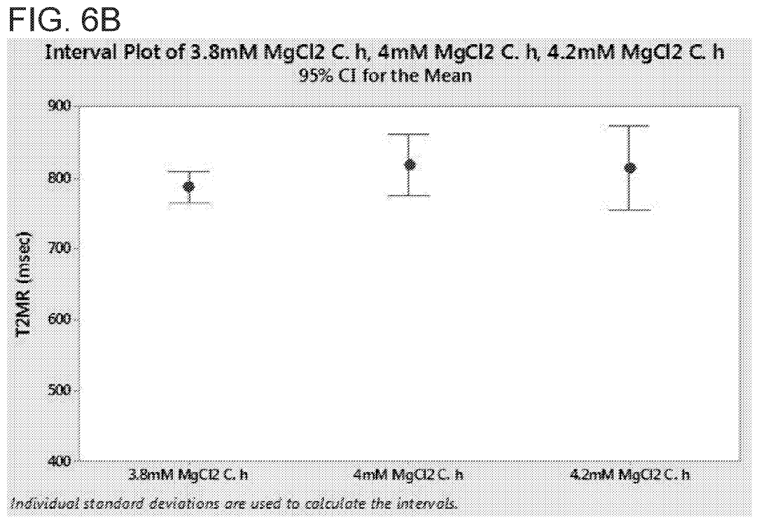

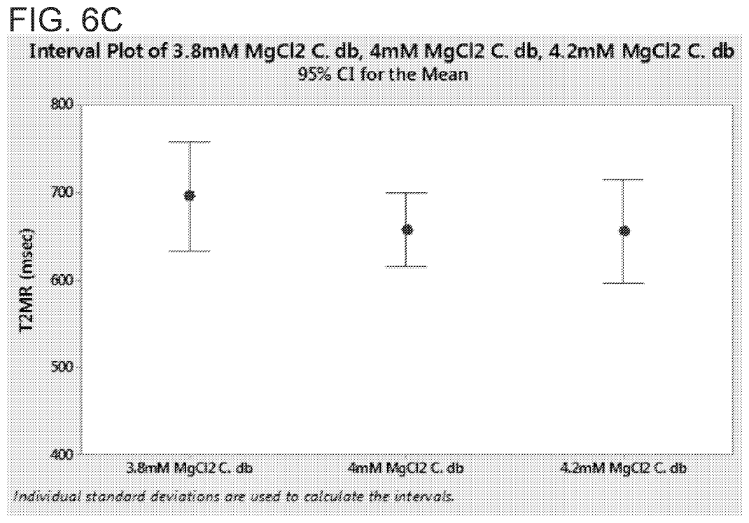

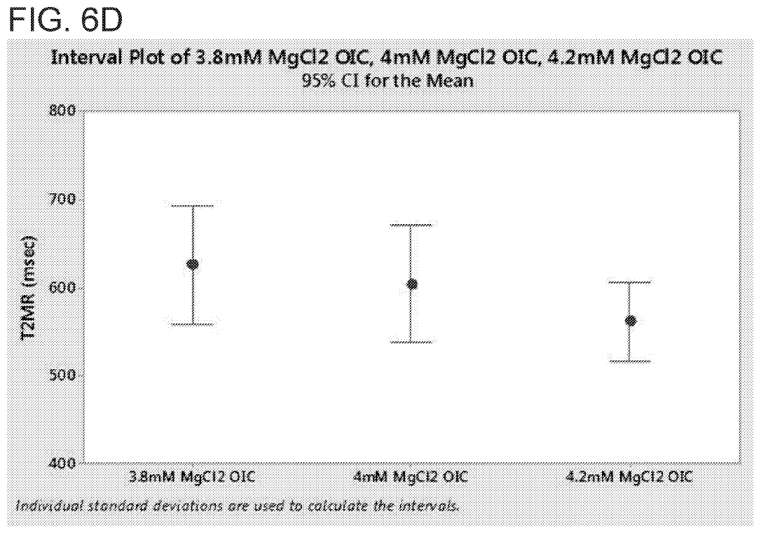

The modular cartridge of the invention is a cartridge that is separated into modules that can be packaged and if necessary sterilized separately. They can also be handled and stored separately, if for example the reagent module requires refrigeration but the detection module does not. FIG. 6 of WO 2012/054639 shows a representative cartridge with an inlet module, a reagent module and a detection module that are snapped together. In this embodiment, the inlet module would be packaged separately in a sterile package and the reagent and detection modules would be pre-assembled and packaged together.

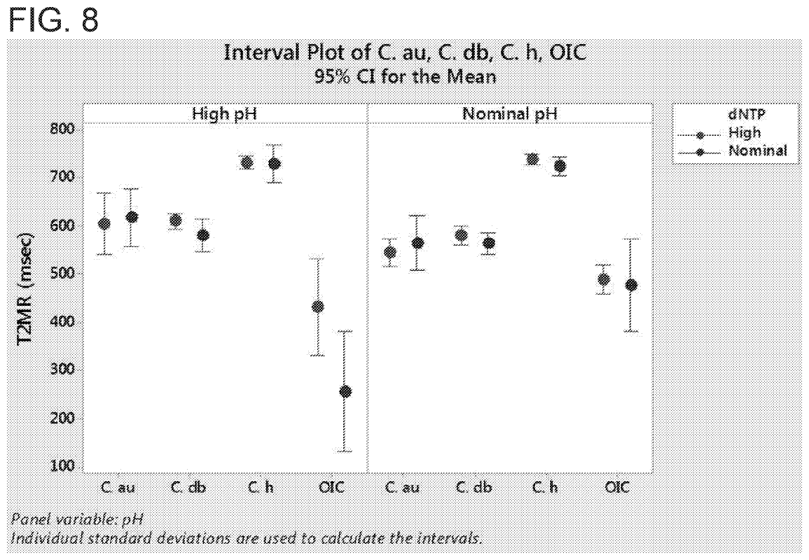

During storage, the reagent module could be stored in a refrigerator while the inlet module could be stored in dry storage. This provides the additional advantage that only a very small amount of refrigerator or freezer space is required to store many assays. At time of use, the operator would retrieve a detection module and open the package, potentially using sterile technique to prevent contamination with skin flora if required by the assay. The Vacutainer tube is then decapped and the inverted inlet module is placed onto the tube as shown in FIG. 7A of WO 2012/054639. This module has been designed to be easily moldable using single draw tooling as shown in FIGS. 7B and 7C of WO 2012/054639 and the top and bottom of the cartridge are sealed with foil to prevent contamination and also to close the channels. Once the tube has been re-sealed using the inlet module, the assembly is turned right side up and snapped onto the remainder of the cartridge. The inlet section includes a well with an overflow that allows sample tubes with between 2 and 6 ml of blood to be used and still provide a constant depth interface to the system automation. It accomplishes this by means of the overflow shown in FIG. 8 of WO 2012/054639, where blood that overflows the sampling well simply falls into the cartridge body, preventing contamination.

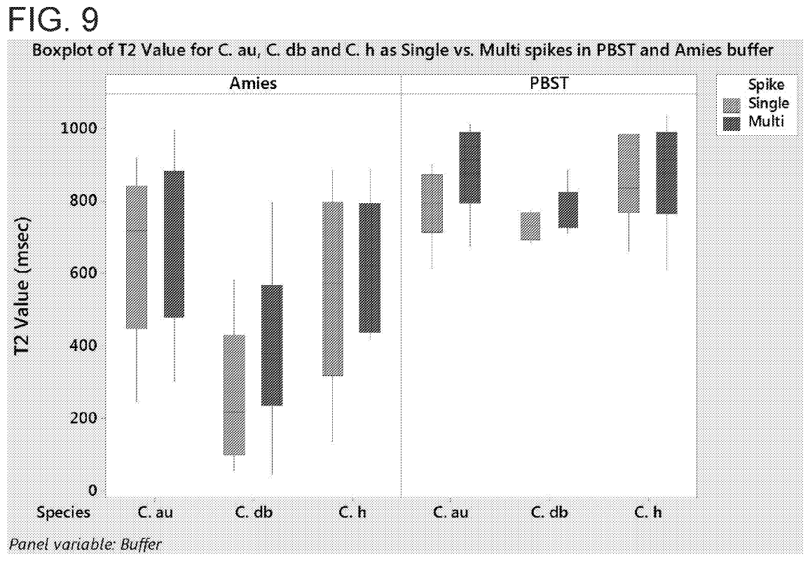

FIGS. 9A-9C of WO 2012/054639 show the means of storing precisely pipetted small volume reagents. The reagents are kept in pipette tips that are shown in FIG. 9C of WO 2012/054639. These are filled by manufacturing automation and then are placed into the cartridge to seal their tips in tight fitting wells which are shown in a cutaway view FIG. 9B of WO 2012/054639. Finally, foil seals are placed on the back of the tips to provide a complete water vapor proof seal. It is also possible to seal the whole module with a seal that will be removed by the operator, either in place of or in addition to the aforementioned foils. This module also provides storage for empty reaction vessels and pipette tips for use by the instrument while the detection module provides storage for capped 200 μl PCR vials used by the instrument to make final measurements from.

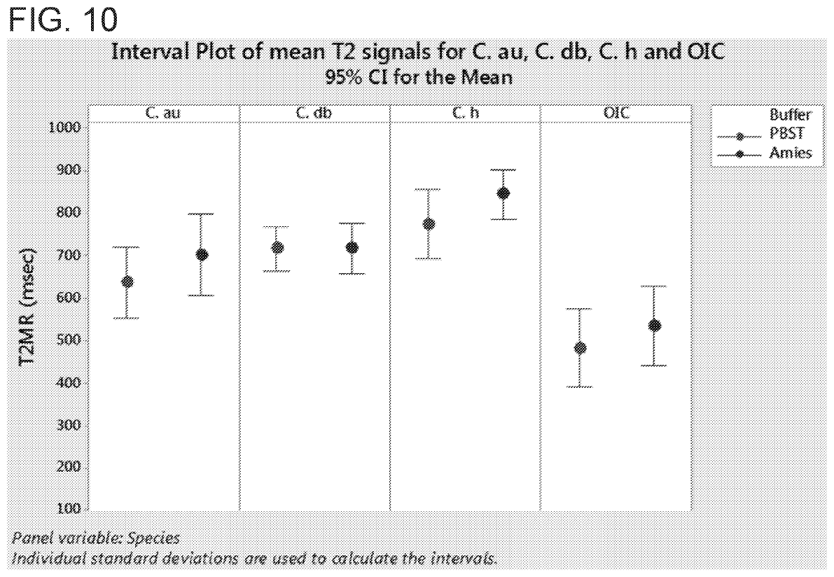

FIGS. 10-13C of WO 2012/054639 show an alternative embodiment of the detection module of the cartridge which is design to provide for contamination control during, for example, pipetting of post-amplification (e.g., PCR) products. This is required because the billion fold amplification produced by DNA amplification (e.g., PCR) presents a great risk of cross contamination and false positives. However, it is desirable to be able to aliquot this mixture safely, because low frequency analytes will have been amplified up and can be distributed for separate detection or identification. There are three ways in which this portion of the cartridge aids in contamination control during this aliquoting operation.

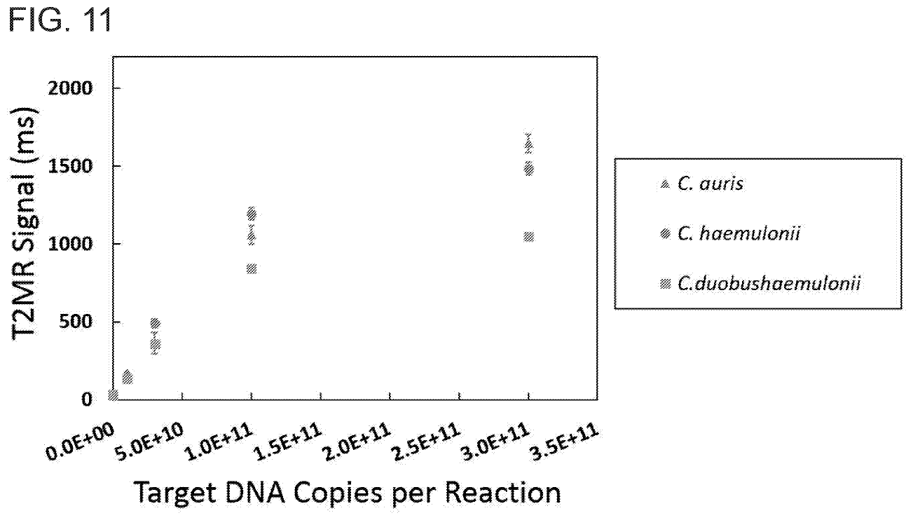

First, the cartridge contains a recessed well to perform the transfer operations in as shown in FIGS. 10A and 10B of WO 2012/054639. Second, the machine provides airflow through this well and down into the cartridge through holes in the bottom of the well, as shown in FIG. 11 of WO 2012/054639. The depth of the well is such that a pipette tip will remain in the airflow and prevent any aerosol from escaping. FIG. 12 of WO 2012/054639 depicts a bottom view of the detection module, showing the bottom of the detection tubes and the two holes used to ensure airflow. An optional filter can be inserted here to capture any liquid aerosol and prevent it from entering the machine. This filter could also be a sheet of a hydrophobic material like GORE-TEX® that will allow air but not liquids to escape. Finally, there is a special seal cap on each 200 ul tube to provide a make then break seal for each pipette tip as it enters the vessel, as shown in FIGS. 13A-13C of WO 2012/054639. It is contemplated that the pipette tip used for aliquoting be stored in this well at all, thus making it possible for the tip never to leave the controlled air flow region.

Alternatively, the modular cartridge is designed for a multiplexed assay. The challenge in multiplexing assays is combining multiple assays which have incompatible assay requirements (i.e., different incubation times and/or temperatures) on one cartridge. The cartridge format depicted in FIGS. 14A-14C of WO 2012/054639 allows for the combination of different assays with dramatically different assay requirements. The cartridge features two main components: (i) a reagent module (i.e., the reagent strip portion) that contains all of the individual reagents required for the full assay panel (for example, a panel as described below), and (ii) the detection module. In some embodiments, a cartridge may be configured to detect from 2 to 24 (e.g., 2, 3, 4, 5, 6, 7, 8, 9, 10, 11, 12, 13, 14, 15, 16, 17, 18, 19, 20, 21, 22, 23, or 24) or more pathogen species (e.g., different Candida species or other microbial pathogens, including bacterial pathogens). The detection modules contain only the parts of the cartridge that carry through the incubation, and can carry single assays or several assays, as needed. The detection module depicted in FIG. 14B of WO 2012/054639 includes two detection chambers for a single assay, the first detection chamber as the control and the second detection chamber for the sample. This cartridge format is expandable in that additional assays can be added by including reagents and an additional detection module.

The operation of the module begins when the user inserts the entire or a portion of the cartridge into the instrument. The instruments performs the assay actuation, aliquoting the assays into the separate detection chambers. These individual detection chambers are then disconnected from the reagent strip and from each other, and progress through the system separately. Because the reagent module is separated and discarded, the smallest possible sample unit travels through the instrument, conserving internal instrument space. By splitting up each assay into its own unit, different incubation times and temperatures are possible as each multiplexed assay is physically removed from the others and each sample is individually manipulated.

The cartridge units of the invention can include one or more populations of magnetic particles, either as a liquid suspension or dried magnetic particles which are reconstituted prior to use. For example, the cartridge units of the invention can include a compartment including from 1×10⁶ to 1×10¹³ magnetic particles (e.g., from 1×10⁶ to 1×10⁸, 1×10⁷ to 1×10⁹, 1×10⁸ to 1×10¹⁰, 1×10⁹ to 1×10¹¹, 1×10¹⁰ to 1×10¹², 1×10¹¹ to 1×10¹³, or from 1×10⁷ to 5×10⁸ magnetic particles) for assaying a single liquid sample.

Panels

The methods, systems, and cartridges of the invention can be configured to detect a predetermined panel of pathogen-associated analytes. For example, the panel can be configured to individually detect one, two, or three of Candida auris, Candida lusitaniae, and Candida haemulonii. These species may be detected using individual target nucleic acids or using target nucleic acids that are universal to all three species, for example, target nucleic acids amplified using universal primers. In some embodiments, the panel is configured to detect Candida auris. In some embodiments, the panel is configured to detect Candida lusitaniae. In some embodiments, the panel is configured to detect Candida haemulonii. In some embodiments, the panel is configured to detect Candida auris and Candida lusitaniae. In some embodiments, the panel is configured to detect Candida auris and Candida haemulonii. In some embodiments, the panel is configured to detect Candida lusitaniae and Candida haemulonii. In some embodiments, the panel is configured to detect Candida auris, Candida lusitaniae and Candida haemulonii.

In some embodiments, the panel can be configured to individually detect one, two, three, four, or all five of Candida auris, Candida lusitaniae, Candida haemulonii, Candida duobushaemulonii, and Candida pseudohaemulonii. These species may be detected using individual target nucleic acids or using target nucleic acids that are universal to all three species, for example, target nucleic acids amplified using universal primers.

In some embodiments, any of the preceding panels is further configured to detect one or more (e.g., 1, 2, 3, 4, 5, 6, or 7) of the following additional Candida species: Candida albicans, Candida guilliermondii, Candida glabrata, Candida krusei, Candida lusitaniae, Candida parapsilosis, and Candida tropicalis). In cases where multiple species of a genus are detected, the species may be detected using individual target nucleic acids or using target nucleic acids that are universal to all of the species, for example, target nucleic acids amplified using universal primers. The panel be detected using the exemplary primers and probes described herein or in WO 2012/054639.

In some embodiments, any of the preceding panels may be further configured to individually detect one or more (e.g., 1, 2, 3, 4, 5, 6, or 7) of Acinetobacter baumannii, Enterococcus faecium, Enterococcus faecalis, Klebsiella pneumoniae, Pseudomonas aeruginosa, Staphylococcus aureus, and Escherichia coli. For example, in some embodiments, the panel is further configured to individually detect Acinetobacter baumannii, Enterococcus faecium, Enterococcus faecalis, Klebsiella pneumoniae, Pseudomonas aeruginosa, Staphylococcus aureus, and Escherichia coli. In particular embodiments, the panel may be further configured to individual detect A. baumannii, E. faecium, K. pneumoniae, P. aeruginosa, E. coli, and S. aureus. The panel can be detected using the exemplary primers and probes described in International Patent Application No. PCT/US2017/014410, which is incorporated by reference herein in its entirety. See, e.g., Example 7 of PCT/US2017/014410.

In any of the above panels, the analyte may be a nucleic acid (e.g., an amplified target nucleic acid, as described above), or a polypeptide (e.g., a polypeptide derived from the pathogen or a pathogen-specific antibody produced by a host subject, for example, an IgM or IgG antibody).

Amplifying Multiple Amplicons Characteristic of a Candida Species for Improved Sensitivity and/or Specificity

In some embodiments, the methods of the invention may involve amplification and detection of more than one amplicon characteristic of a Candida species. In some embodiments, amplification of more than one target nucleic acid characteristic of a species increases the total amount of amplicons characteristic of the species in an assay (in other words, the amount of analyte is increased in the assay). This increase may allow, for example, an increase in sensitivity and/or specificity of detection of the species compared to a method that involves amplification and detection of a single amplicon characteristic of a species. In some embodiments, the methods of the invention may involve amplifying 2, 3, 4, 5, 6, 7, 8, 9, or 10 amplicons characteristic of a species. In some embodiments, the species is selected from Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, and C. tropicalis.

In some embodiments, multiple (e.g., 2, 3, 4, 5, 6, 7, 8, 9, or 10) single-copy loci from a Candida species are amplified and detected. In some embodiments, 2 single-copy loci from a species are amplified and detected. In some embodiments, amplification and detection of multiple single-copy loci from a Candida species (e.g., Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, or C. tropicalis) may allow for a sensitivity of detection comparable with methods that involve detecting an amplicon that is derived from a multi-copy locus. In some embodiments, methods involving detection of multiple single-copy loci amplified from a Candida species can detect from about 1-10 cells/mL (e.g., 1, 2, 3, 4, 5, 6, 7, 8, 9 or 10 cells/mL) of the Candida species in a liquid sample. In some embodiments, methods involving detection of multiple single-copy loci amplified from a Candida species have at least 95% correct detection when the microbial species is present in the liquid sample at a frequency of less than or equal to 5 cells/mL (e.g., 1, 2, 3, 4, or 5 cells/mL) of liquid sample.

The invention also provides embodiments in which at least three amplicons are produced by amplification of two target nucleic acids, each of which is characteristic of a Candida species. For example, in some embodiments, a first target nucleic acid and a second target nucleic acid to be amplified may be separated (for example, on a chromosome or on a plasmid) by a distance ranging from about 50 base pairs to about 1000 1500 base pairs (bp), e.g., about 50, 100, 150, 200, 250, 300, 400, 500, 600, 700, 800, 900, or 1000, 1100, 1200, 1300, 1400, or 1500 bp base pairs. In some embodiments, a first target nucleic acid and a second target nucleic acid to be amplified may be separated (for example, on a chromosome or on a plasmid) by a distance ranging from about 50 bp to about 1000 bp (e.g., about 50, 100, 150, 200, 250, 300, 400, 500, 600, 700, 800, 900, or 1000 bp). In some embodiments the first target nucleic acid and the second target nucleic acid to be amplified may be separated by a distance ranging from about 50 bp to about 1500 bp, from about 50 bp to about 1400 bp, from about 50 bp to about 1300 bp, from about 50 bp to about 1200 bp, from about 50 bp to about 1100 bp, from about 50 bp to about 1000 bp, from about 50 bp to about 950 bp, from about 50 bp to about 900 bp, from about 50 bp to about 850 bp, from about 50 bp to about 800 bp, from about 50 bp to about 800 bp, from about 50 bp to about 750 bp, from about 50 bp to about 700 bp, from about 50 bp to about 650 bp, from about 50 bp to about 600 bp, from about 50 bp to about 550 bp, from about 50 bp to about 500 bp, from about 50 bp to about 500 bp, from about 50 bp to about 450 bp, from about 50 bp to about 400 bp, from about 50 bp to about 350 bp, from about 50 bp to about 300 bp, from about 50 bp to about 250 bp, from about 50 bp to about 200 bp, from about 50 bp to about 150 bp, or from about 50 bp to about 100 bp. In some embodiments, amplification of the first and second target nucleic acids using individual primer pairs (each having a forward and a reverse primer) may lead to amplification of an amplicon that includes the first target nucleic acid, an amplicon that includes the second target nucleic acid, and an amplicon that contains both the first and the second target nucleic acid. This may result in an increase in sensitivity of detection of the species compared to samples in which the third amplicon is not present. In any of the preceding embodiments, amplification may be by asymmetric PCR.

The invention features magnetic particles decorated with nucleic acid probes to detect two or more amplicons characteristic of a Candida species. For example, in some embodiments, the magnetic particles include two populations, wherein each population is conjugated to probes such that the magnetic particle that can operably bind each of the two or more amplicons. For instance, in embodiments where two target nucleic acids have been amplified to form a first amplicon and a second amplicon, a pair of particles each of which have a mix of capture probes on their surface may be used. In some embodiments, the first population of magnetic particles may be conjugated to a nucleic acid probe that operably binds a first segment of the first amplicon and a nucleic acid probe that operably binds a first segment of the second amplicon, and the second population of magnetic particles may be conjugated to a nucleic acid probe that operably binds a second segment of the first amplicon and a nucleic acid probe that operably binds a second segment of the second amplicon. For instance, one particle population may be conjugated with a 5′ capture probe specific to the first amplicon and a 5′ capture probe specific to second amplicon, and the other particle population may be conjugated with a 3′ capture probe specific to the first amplicon and a 3′ capture probe specific to the second amplicon.

In such embodiments, the magnetic particles may aggregate in the presence of the first amplicon and aggregate in the presence of the second amplicon. Aggregation may occur to a greater extent when both amplicons are present.

In some embodiments, a magnetic particle may be conjugated to two, three, four, five, six, seven, eight, nine, or ten nucleic acid probes, each of which operably binds a segment of a distinct target nucleic acid. In some embodiments, a magnetic particle may be conjugated to a first nucleic acid probe and a second nucleic acid probe, wherein the first nucleic acid probe operably binds to a first target nucleic acid, and the second nucleic acid probe operably binds to a second target nucleic acid. In other embodiments, a magnetic particle may be conjugated to a first nucleic acid probe that operably binds a first target nucleic acid, a second nucleic acid probe that operably binds a second target nucleic acid, and a third nucleic acid that operably binds a third target nucleic acid. In yet other embodiments, a magnetic particle may be conjugated to a first nucleic acid probe that operably binds a first target nucleic acid, a second nucleic acid probe that operably binds a second target nucleic acid, a third nucleic acid that operably binds a third target nucleic acid, and a fourth nucleic acid probe that operably binds a fourth target nucleic acid. In still other embodiments, a magnetic particle may be conjugated to a first nucleic acid probe that operably binds a first target nucleic acid, a second nucleic acid probe that operably binds a second target nucleic acid, a third nucleic acid that operably binds a third target nucleic acid, a fourth nucleic acid probe that operably binds a fourth target nucleic acid, and a fifth nucleic acid probe that operably binds a fifth target nucleic acid. In some embodiments, one population of magnetic particles includes the 5′ capture probe for each amplicon to be detected, and the other population of magnetic particles includes the 3′ capture probe for each amplicon to be detected.

### EXAMPLES

The following examples are put forth so as to provide those of ordinary skill in the art with a complete disclosure and description of how the devices, systems, and methods described herein are performed, made, and evaluated, and are intended to be purely exemplary of the invention and are not intended to limit the scope of what the inventors regard as their invention.

### Example 1: Multiplex Assay for Detection of Candida Species, Including Candida auris, Candida lusitaniae, and Candida haemulonii

A rapid, accurate, and reproducible molecular diagnostic test was developed for the detection of three Candida species (Candida auris, Candida lusitaniae, and Candida haemulonii) directly within human whole blood with a limit of detection (LOD) of 10 cells/mL or less and a time to result of less than 4 hours.

A multiplex assay targeting Candida auris, Candida lusitaniae, and Candida haemulonii was developed using cultured cells spiked in K₂EDTA anticoagulated blood from healthy human donors. Automated cell counting was used to determine the concentration of an overnight culture of C. auris. From this stock, the culture was diluted to target titer, and inoculated into whole blood, followed by confirmation plating to confirm cell titer. 4 mL spiked blood samples were processed on a T2DX® instrument (T2 Biosystems, Inc.), which automates the following steps: chemical lysis of blood cells, concentration of target cells through centrifugation, release of target cell DNA through mechanical lysis (bead beating), and amplification of target DNA. The T2DX® instrument detects and identifies the presence of each individual species by hybridizing the amplicon with DNA probe conjugated superparamagnetic particles. The particles only cluster only in the presence of the species they are directed against, and the resulting clustering is identified by the T2 relaxation signal. Amplicon-induced clustering caused by target specific DNA resulted in 30× higher T2MR signal versus dispersed particles allowing for sensitive detection and identification of Candida species direct from whole blood. An analogous manually processed assay (manual assay) was also used during development of the assay.

Materials and Methods

The assay workflow can include those described in Examples 22 and 25 of WO 2012/054639, as described further below. The assay was performed by a T2DX® instrument or manually with essentially the same results.

Magnetic Resonance Relaxometer

A compact magnetic resonance (MR) system was used for precise T2 relaxation measurements in order to perform the intended assay under the described conditions. This system was held at 37° C. via temperature control and contains a permanent magnet of approximately 0.5 T, corresponding to a proton frequency of operation of 22-24 MHz. All standard MR components: radio frequency probe, low-noise pre-amplifier and transmitter electronics, spectrometer board, as well as the temperature control hardware are packaged in the system. The system uses standard AC power input and connects to an external computer via Ethernet. A user friendly graphical user interface allows users to set experimental parameters.

The system has been designed to accept samples in standard 0.2 ml PCR tubes. The electronics as well as the coil were optimized to improve the measurement precision of the applicable sample volumes, allowing us to achieve single-scan run to run CVs in T2 of less than 0.1%. Instrument to instrument variability is under 2% with minimal tolerance requirements on the system components and without calibration.

Nanoparticle Sensor Conjugation and Characterization

800 nm carboxylated iron oxide superparamagnetic particles, consisting of numerous iron oxide nanocrystals embedded in a polymer matrix including a total particle diameter of 800 nm (see Demas et al., New J. Phys. 13: 1 (2011)), were conjugated to animated DNA oligonucleotides using standard carbodiimide chemistry. Oligonucleotide derivatized particles are then subjected to a functional performance test by conducting hybridization induced agglomeration reactions using diluted synthetic oligonucleotide targets identical in sequence to the fungal ITS2 sequences from the three different Candida species within a sodium phosphate hybridization buffer 4×SSPE (600 mM NaCl, 40 mM sodium phosphate, 4 mM EDTA). Reversibility of the agglomeration reaction was confirmed by subjecting agglomerated reactions to a 95° C. heat denaturation step, conducting a T2 measurement, and repeat hybridization at 62° C. followed by a second T2 measurement.

Preparation of single probe particles: 800 nm carboxylated iron oxide superparamagnetic particles, consisting of numerous iron oxide nanocrystals embedded in a polymer matrix including a total particle diameter of 800 nm (see Demas et al., New J. Phys. 13: 1 (201 1)) were washed using a magnetic rack prior to use. The magnetic particles were resusupended in 215 μL 55 mM MES buffer pH 6, and 10 μL of aminated probe (obtained from IDT), at 1 mM concentration per mg of particle to be prepared. A 3′ aminated probe particle and a 5′ aminated probe particle were prepared (e.g., the probe for C. parapsilosis). The probe was added to the particle and the suspension was vortexed using a vortexer equipped with a foam holder to hold the tube. The vortexer was set to a speed that keeps the particles well-suspended without any splashing. N-ethyl-N′-(3-dimethylaminopropyl) carbodiimide hydrochloride (EDC) was then dissolved in water and immediately added to the vortexing particle-probe mixture. The tube was then closed and incubated with rotation in an incubator at 37° C. for 2 hours. The tube was then placed in a magnetic rack and the reaction fluid was removed. The particles were washed with a series of washes as follows: 55 mM MES buffer pH 6, 0.1 M sodium bicarbonate, pH 8.0 with a 5 minute incubation at RT. The particles were then subjected to a heat-stress at 60-65° C. in 8×SSPE, 0.1% tween 20 with rotation. In the Candida assay, the particles are diluted in 8×SSPE, 0.1% TWEEN® 20 supplemented with PROCLIN® 950 as a preservative.

PCR Primer and Nanoparticle Capture Probe Design

Universal Pan Candida PCR primers were designed complementary to 5.8S and 26S rRNA sequences that amplify the intervening transcribed spacer 2 (ITS2) region of the Candida genome. A pair of oligonucleotide capture probes was designed complementary to nested sequences at the 5′ and 3′ end respectively of the asymmetrically amplified PCR product. The capture probe that hybridizes to the 5′ end of the amplicon was 3′ aminated while the capture probe that hybridizes to the 3′ end of the amplicon was 5′ aminated. A poly-T linker (n=9 to 24) is added between the amino group and the first nucleotide base of the capture probe sequence. HPLC purified PCR primers and capture probes were procured from IDT Technologies (Coralville, Iowa).

In the present example, a forward primer having the oligonucleotide sequence 5′-GGC ATG CCT GTT TGA GCG T-3′ (SEQ ID NO: 1) or GGG CAT GCC TGT TTG AGC GT-3′ (SEQ ID NO: 2) and a reverse primer having the oligonucleotide sequence 5′-GCT TAT TGA TAT GCT TAA GTT CAG CGG GT-3′ (SEQ ID NO: 3) were used.

The capture probes listed in Table 5 were used to detect the presence of the indicated Candida species.

TABLE 5
Capture Probes for Detection of Candida auris, Candida lusitaniae, and Candida
haemulonii

| Candida Capture Probes | Sequence |
|---|---|
| Candida auris 5′ Capture probe | CTA CCT GAT TTG AGG CGA CAA CAA AAC (SEQ ID NO: 4) |
| Candida auris 3′ Capture probe | CCG CGA AGA TTG GTG AGA AGA CAT (SEQ ID NO: 5) |
| Candida lusitaniae 5′ Capture | CCT ACC TGA TTT GAG GGC GAA ATG TC (SEQ ID NO: 6) |
| probe | |
| Candida lusitaniae 3′ Capture | GGA GCA ACG CCT AAC CGG G (SEQ ID NO: 7) |
| probe | |
| Candida haemulonii 5′ Capture | GTC CTA CCT GAT TTG AGG GGA AAA AGC (SEQ ID NO: 8) |
| probe | |
| Candida haemulonii 3′ Capture | AAC AAA TCC ACC AAC GGT GAG AAG ATA T (SEQ ID NO: 9) |
| probe | |

The capture probes listed in Table 6 can also be used to detect the presence of the indicated Candida species using the same assay as described in this Example.

TABLE 6
Capture Probes for Detection of Candida duobushaemulonii and Candida
pseudohaemulonii.

| | |
|---|---|
| Candida duobushaemulonii | GCG TAG ACT TCG CTG CGG AT (SEQ ID NO: 28) |
| 5′ Capture probe | |
| Alternate Candida | CGT AGA CTT CGC TGC GGA T (SEQ ID NO: 48) |
| duobushaemulonii 5′ | |
| Capture probe | |
| Candida duobushaemulonii | CTG GGC GGT GAG AAG AAA TC (SEQ ID NO: 29) |
| 3′ Capture probe | |
| Candida pseudohaemulonii | GCG TAG ACT TCG CTG CTG GAA (SEQ ID NO: 30) |
| 5′ Capture probe | |
| Candida pseudohaemulonii | CCG TGC GGT GAG AAG AAA TC (SEQ ID NO: 31) |
| 3′ Capture probe | |
| Candida duobushaemulonii | TCC TAC CTG ATT TGA GGA AAT AGC ATG G (SEQ ID NO: 32) |
| and Candida | |
| pseudohaemulonii (dual | |
| target) 5′ Capture probe | |
| Candida duobushaemulonii | ATT TAG CGG ATG CAA AAC CAC C (SEQ ID NO: 33) |
| and Candida | |
| pseudohaemulonii (dual | |
| target) 3′ Capture probe | |

Candida Cultivation and In-Vitro Spiked Sample Preparation

Candida auris (strain AR-0390), C. krusei (strain AR-0397), Candida haemulonii (strain AR-0393) and Candida lusitaniae (strain AR-0398) were obtained from the Center for Disease Control Candida auris panel (NCEZID, Atlanta, Ga.) and used to prepare the in vitro spiked whole blood specimens. Candida strains were cultivated on yeast peptone dextrose (YPD) agar plates and incubated at 30° C. for 24 hours. Well-isolated, single colonies were selected and suspended in YPD medium in a baffled, vented flask and incubated on a shaking platform for 24 hours at 30° C. The cell concentration was determined using an automated cell counter (Nexcelom Bioscience) following the manufacturer's instruction. Candida cells were serially diluted in 1×PBS, pH 7.4 (Invitrogen) to concentrations ranging from 1000 to 100 cells/mL. To prepare spiked samples fresh K₂EDTA-treated, whole human blood was spiked as a bulk volume with 10 μL per mL of blood with the appropriate cell dilution. The solution was mixed by gently inverting the bottle and 4 mL aliquots were transferred using sterile technique to untreated, 4 mL vacutainers (Becton Dickinson) and samples stored at 2-8° C. prior to testing within 24 hours of spike. Confirmation of spike titers was confirmed by plating of the cell dilution on YPD agar plates and manual counting of colonies after incubation at 30° C. for 26±4 hours.

Whole Blood PCR

Erythrocyte lysis was conducted within 2 mL of the whole blood sample using previously described methods (see Bramley et al., Biochimica et Biophysica Acta (BBA)—Biomembranes, 241:752 (1971) and Wessels J M, Biochim Biophys Acta., 2: 178 (1973)), a low speed centrifugation is then conducted and the supernatant was removed and discarded. 100 μL of Tris EDTA (TE) buffer pH 8.0 containing 400 copies of the inhibition control was then added to the harvested pellets and the suspension was subjected to mechanical lysis (see Garver et al., Appl. Microbiol., 1959. 7:318 (1959); Hamilton et al., Appl. Microbiol., 10: 577 (1962); and Ranhand, J. M., Appl. Microbiol., 28:66 (1974)). 50 μL of lysate was then added to 50 μL of an asymmetric PCR master mix containing deoxynucleotides, PCR primers and a whole blood compatible thermophilic DNA polymerase (T2 Biosystems, Lexington, Mass.). Thermocycling was conducted using the following cycle parameters: heat denaturation at 95° C. for 10 minutes, 40 cycles consisting of a 20 second 95° C. heat denaturation step, a 20 second 62° C. annealing step, and a 30 second 68° C. elongation step, and a final extension at 68° C. for 10 minutes.

In some instances, the limit of detection is improved by washing the pellet. In this approach, 2.0 mL of whole blood was combined with 100 μL of TRAX erythrocyte lysis buffer (i.e., a mixture of nonyl phenoxy-polyethoxylethanol (NP-40) and 4-octylphenol polyethoxylate (TRITON®-X 100)) and incubated for about 5 minutes. The sample was centrifuged for 5 min at 6000 g and the resulting supernatant was removed and discarded. To wash the pellet, the pellet was mixed with 200 μL of Tris EDTA (TE) buffer pH 8.0 and subjected to vortexing. The sample was again centrifuged for 5 minutes at 6000 g and the resulting supernatant was removed and discarded. Following the wash step the pellet was mixed with 100 μL TE buffer and subjected to bead beating (e.g., such as with 0.5 mm glass beads, 0.1 mm silica beads, 0.7 mm silica beads, or a mixture of differently sized beads) with vigorous agitation. The sample was again centrifuged. 50 μL of the resulting lysate was then added to 50 μL, of an asymmetric PCR master mix containing a deoxynucleotides, PCR primers and a whole blood compatible thermophilic DNA polymerase (T2 Biosystems, Lexington, Mass.). Thermocycling and hybridization induced agglomeration assays were conducted as described herein to produce T2 values characteristic of the presence of Candida in the blood sample. The assay can produce (i) a coefficient of variation in the T2 value of less than 20% on Candida positive samples; (ii) at least 95% correct detection at less than or equal to 5 cells/mL in samples spiked into patient blood samples.

Hybridization Induced Agglomeration Assays

Fifteen microliters of the resulting amplification reaction was aliquoted into 0.2 mL thin walled PCR tubes and incubated within a sodium phosphate hybridization buffer (4×SSPE) with pairs of oligonucleotide derivatized nanoparticles at a final iron concentration of 0.2 mM iron per reaction.

Hybridization reactions were incubated for 30 minutes at 62° C. within a shaking incubator set at an agitation speed of 1400 rpm (Vortemp, LabNet International).

Hybridized samples are then placed in a 37° C. heating block to equilibrate the temperature to that of the MR reader for 3 minutes. Each sample is then subjected to a 5 second vortexing step (3000 rpm) and inserted into the MR reader for T2 measurement.

Results

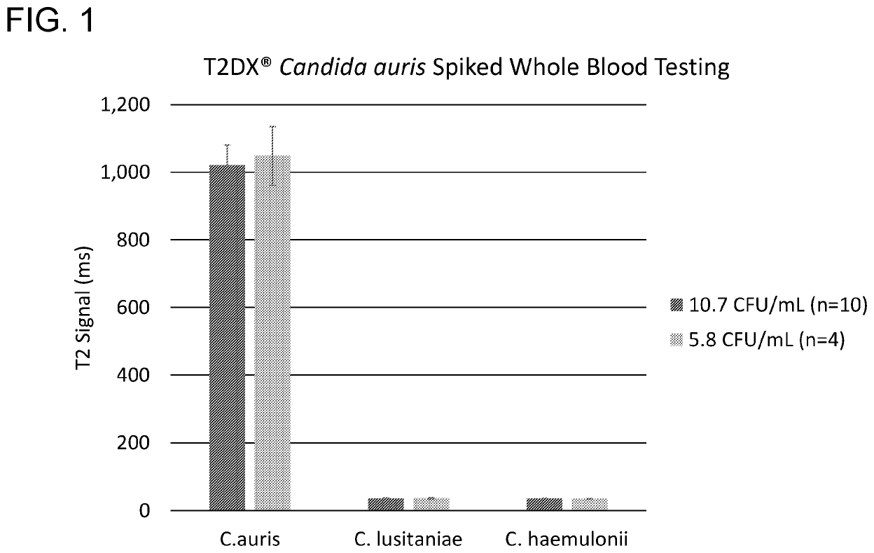

Sensitive and specific detection of Candida auris was achieved direct from blood in less than 4 hours without blood culture on the T2DX® instrument and in an analogous manual assay. A Limit of Detection (LoD) for C. auris was demonstrated to be ≤10 CFU/mL (see FIG. 1 and Table 7). T2MR signals of samples spiked with target were approximately 30 times higher than samples with no target present.

TABLE 7
Limit of Detection Data for Candida auris

| | C. auris | C. auris | C. auris | Whole Blood |
|---|---|---|---|---|
| | 6 CFU/mL (n = 4) | 10 CFU/mL (n = 10) | 21 CFU/mL (n = 6) | Blank (n = 4) |
| Average T2 | 1048 | 1042 | 991 | 33 |
| Signal (ms) | | | | |
| Standard | 87 | 59 | 53 | 3 |
| Deviation | | | | |
| CV % | 8 | 6 | 5 | 8 |

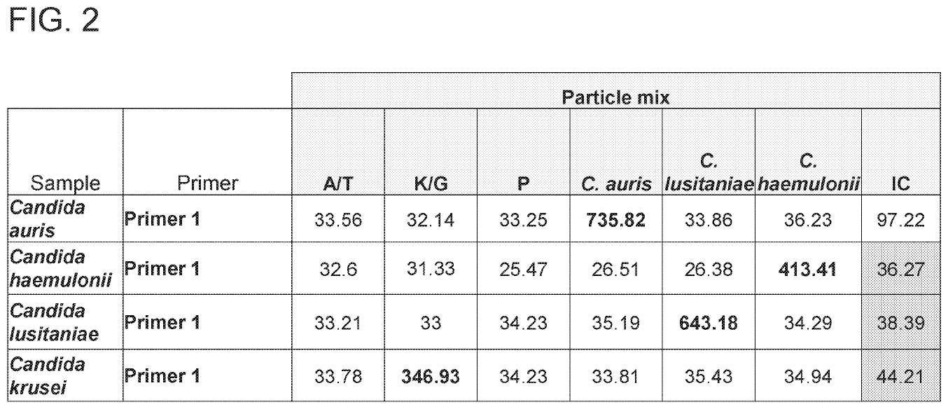

Additionally, no cross reactivity was observed between Candida auris, Candida lusitaniae, and Candida haemulonii. In particular, in a manual assay of unquantified cell culture (very high cell concentration) in whole blood, using the assay described above, Candida auris, Candida lusitaniae, and Candida haemulonii were each detected using the appropriate magnetic particles (FIG. 2). In contrast, no cross reactivity was observed using the probes described above or probes described in WO 2012/054639 (FIG. 2).

In conclusion, low concentrations (≤10 CFU/mL) of Candida cells (e.g., Candida lusitaniae, Candida haemulonii, Candida duobushaemulonii, and Candida pseudohaemulonii) can reliably be detected and identified in whole blood samples by T2MR. This assay allows for the rapid screening and identification of patients infected with Candida auris, aiding in the hospital management and targeted therapy of this emerging multidrug resistant pathogen.

### Example 2: Primer Testing

In silico analysis identified that C. auris and C. haemulonii have a mismatch at the terminal 3′ residue (FIG. 12) that prevents priming of target sequences using the Candida forward primer of SEQ ID NO: 10 in the formulation of Reagent A (forward primer, reverse primer, Tris EDTA, MgCl₂, glycerol, tricine, and ammonium sulfate; see WO 2017/180745, which is incorporated herein by reference in its entirety). Three forward primers were designed and tested: (1) Short—a shortened forward primer with the 3′ mismatch base removed, (2) a nitroindole modification replacing the terminal mismatch (“Nitro”), and (3) a deoxylnosine modification replacing the terminal mismatch (“deoxyl”). Primer sequences are shown in Table 8 below. All amplifications reactions utilized the Candida reverse primer (SEQ ID NO: 3). High and low titer C. auris and C. krusei spikes in blood were processed by the manual assay and amplifications were done using the different primers. All the assays were processed with Candida IC. The testing with C. auris genomic DNA was carried out with Orange IC (OIC).

TABLE 8
Primer Sequences tested for the T2Cauris Panel

| Primer | Sequence |
|---|---|
| Candida F | GGC ATG CCT GTT TGA GCG TC |
| | (SEQ ID NO: 10) |
| Candida F | GGC ATG CCT GTT TGA GCG T |
| Short | (SEQ ID NO: 1) |
| Candida F 3 | GGC ATG CCT GTT TGA GCG T/35NiTInd/ |
| Nitro | (SEQ ID NO: 46) |
| Candida F | GGC ATG CCT GTT TGA GCG T/3deoxyl/ |
| deoxyl | (SEQ ID NO: 47) |
| Candida R | GCT TAT TGA TAT GCT TAA GTT CAG CGG GT |
| | (SEQ ID NO: 3) |

Methods

High titer cell spikes were prepared by scraping a colony of the respective species and spiking into blood. Low titer cell spikes were prepared in blood aimed at 12.5 CFU/mL. The low titer spikes were later quantified as 0.2 CFU/mL for C. auris and 2.8 CFU/mL for C. krusei. Three different isolates of C. auris and one isolate each of C. haemulonii, C. krusei, and C. lusitaniae were used for this screening. These were tested by manual assay. OIC was tested with C. auris gDNA in buffer.

Results

A summary of the results are presented in Table 9. High titers of C. auris and C. krusei were not detected using Candida Reagent A. Removing the terminal C residue from the Candida Forward primer or changing the terminal C residue to the universal base deoxylnosine resulted in amplification and detection of C. auris. The universal base nitroindole did not exhibit an improvement in performance in this assay. C. lusitaniae and C. krusei do not have a mismatch with the terminal C nucleotide of the Candida F primer and both had good performance with Reagent A. The short primer or deoxylnosine did not cause reduction of T2 signal for C. krusei at high titers. Low Candida IC signals were postulated to be the result of competitive inhibition due to the high titer spikes. No cross-reactivity between any probes was noticed, indicating the probe designs are sufficiently selective.

The low titer spikes indicated that the deoxylnosine-terminated primer did not amplify Candida IC sufficient for detection, indicating against competitive inhibition caused by high titer spikes. The short primer amplified the Candida IC. However, the Candida IC amplified with the standard Candida primers yielded higher T2 signals. C. auris was detected with both the short and deoxylnosine primers, although the modest hit rates were most likely due to very low titer samples (0.2 CFU/mL). C. krusei performed well with both the nominal and the short primer mixes but did not amplify with the deoxylnosine primer mix. The deoxylnosine may have less sensitivity than the shorter primer and may not amplify the T2 Candida IC sequence. Because the assay is intended to detect low prevalence Candida species in whole blood or buffer, the potential for IC invalids due to competitive inhibition of IC is high. Therefore, Orange IC (OIC) was explored as a potential Internal Control for the T2Cauris assay. When co-amplifying with 50 copies of the C. auris gDNA, OIC performed well and was used in subsequent studies. In Table 9, “K/G” refers to C. krusei/C. glabrata detection particles.

TABLE 9
Testing of different Candida forward primers
with three different isolates of C. auris

| | | Primer Mix (Avg T2MR) | | | |
|---|---|---|---|---|---|
| | Detection | Reagent | Short | CF | CF |
| | Particle | A | Primer | Deoxyl | Nitro |
| High Concentration Cell | | | | | |
| Spike Target | | | | | |
| C. auris Isolate 1 | C. auris | 32 | 800 | 705 | 30 |
| | CIC | 727 | 319 | 32 | 33 |
| C. auris Isolate 2 | C. auris | 110 | 664 | 741 | 29 |
| | CIC | 781 | 74 | 33 | 24 |
| C. auris Isolate 3 | C. auris | 43 | 736 | 811 | 31 |
| | CIC | 429 | 97 | 33 | 33 |
| C. haemulonii | C. haemulonii | 58 | 413 | 646 | 30 |
| | CIC | 671 | 36 | 34 | 34 |
| C. krusei | K/G | 323 | 347 | 404 | 30 |
| | CIC | 43 | 44 | 33 | 34 |
| C. lusitaniae | C. lusitaniae | 630 | 643 | 170 | 32 |
| | CIC | 37 | 38 | 33 | 33 |
| Low Concentration Cell | | | | | |
| Spike | | | | | |
| C. auris Isolate 1 | C. auris | 33 | 72 | 56 | NT |
| (>1CFU/mL) | | | | | |
| | CIC | 529 | 217 | 35 | NT |
| C. krusei (2.8CFU/mL) | K/G | 308 | 166 | 32 | NT |
| | CIC | 325 | 159 | 35 | NT |
| Genomic DNA Spike | | | | | |
| C. auris 50 | C. auris | NT | 515 | NT | NT |
| copies/Reaction | OIC | NT | 438 | NT | NT |
| OIC 300 copies/Reaction | | | | | |

Conclusion

These data demonstrate that the Candida Short Forward primer (SEQ ID NO: 1) and the Candida Reverse primer (SEQ ID NO: 3) can amplify target nucleic acids from Candida species including C. auris, C. haemulonii, C. krusei, C. lusitaniae, and C. duobushaemulonii (see also Example 7). Additionally, the OIC can be used as an internal control in this assay.

### Example 3: Cross-Reactivity and Competitive Inhibition

A cross-reactivity study was performed to determine whether species specific particles are reactive with nearest neighbor species. A competitive inhibition study was executed to determine if the presence of high levels of another Candida species impaired the sensitivity of the other panel members.

Methods

Probes were designed specific to the species listed in Table 10 below. These were tested with spiked whole blood or swab buffers processed either by manual assay or on the T2DX® instrument (T2 Biosystems, Inc.).

TABLE 10
Results of the cross-reactivity testing of the T2Cauris panel

Whole blood

| Target in | T2MR Detection Channel (Positive/No. of samples run) | | | | | | |
|---|---|---|---|---|---|---|---|
| Spike >750 | | | | | C. albicans/ | C. krusei/ | |
| CFU/mL | C. auris | C. h | C. db | C. lusitaniae | C. tropicalis | C. glabrata | C. parapsilosis |
| C. auris (I/II/III) | 12/12 | 0/12 | 0/12 | 0/12 | 0/12 | 0/12 | 0/12 |
| C. haemulonii | 0/4 | 4/4 | 0/4 | 0/4 | 0/4 | 0/4 | 0/4 |
| C. krusei | 0/4 | 0/4 | 0/4 | 0/4 | 0/4 | 4/4 | 0/4 |
| C. lusitaniae | 0/4 | 0/4 | 0/4 | 4/4 | 0/4 | 0/4 | 0/4 |
| C. db | 0/7** | 0/7** | 7/7** | 0/4 | 0/4* | 0/4* | 0/4* |

(*T2DX ® data, **Manual + T2DX ® data)

Results

No cross-reactivity was observed when species specific particles were hybridized with amplicon generated with high titer cell spikes from nearest neighbor species (Table 10).

Conclusions

Species-specific particles did not cross react with the species tested. Higher titer spikes of Candida glabrata competitively inhibited C. auris, leading to lower T2 signals, but did not cause dropouts at the concentrations tested. Additionally, the clinical relevance of these concentrations is not known.

### Example 4: Evaluation of DNA Polymerase Enzymes for T2Cauris Assay

The performance of a number of DNA polymerases was evaluated in the context of the T2Cauris assay. Amplification was performed with 25 copies of C. auris gDNA per reaction in OIC buffer amplified on a MASTERCYCLER® thermal cycler using five distinct DNA polymerases at different concentrations. All enzymes tested amplified C. auris and OIC (Table 11). Higher concentrations of Taq polymerases yielded higher T2MR signals for both C. auris and OIC (Table 11). These data serve as a proof of principle that a number of DNA polymerase enzymes can be used in buffer-based T2MR assays. For the remainder of the experiments described herein, the 0.5× HS enzyme was used.

TABLE 11
Testing of Different DNA Polymerase Enzymes

| | | | | | | | | | | | | | | | Formulated Enzyme | |
|---|---|---|---|---|---|---|---|---|---|---|---|---|---|---|---|---|
| | 1x Taq | | 5x Taq | | 1x HS Tag | | 5x HS Tag | | 1x AptaTaq | | 5x AptaTaq | | 0.5x HS Enzyme | | (Candida) | |
| | C. au | OIC | C. au | OIC | C. au | OIC | C. au | OIC | C. au | OIC | C. au | OIC | C. au | OIC | C. au | OIC |
| Avg T2 | 301 | 34 | 839 | 314 | 232 | 29 | 851 | 108 | 421 | 35 | 831 | 193 | 707 | 399 | 811 | 102 |
| (msec) | | | | | | | | | | | | | | | | |
| St. Dev. | 149 | 5 | 30 | 18 | 124 | 1 | 64 | 33 | 66 | 4 | 15 | 38 | 99 | 62 | 27 | 41 |
| % CV | 49% | 15% | 4% | 6% | 53% | 4% | 7% | 30% | 16% | 11% | 2% | 20% | 14% | 16% | 3% | 41% |

### Example 5: Analytical Sensitivity in Blood

The analytical sensitivity of the assay for detection of Candida auris in blood was evaluated. The assay essentially as described in Example 1 was used to detect Candida auris in spiked blood samples on the T2DX® instrument at 5.3, 10.7, and 21.3 CFU/mL. QC plating was confirmed at 5.3, 10.7 and 21.3 CFU/mL. 100% detection was observed at all titer levels (Table 12). These results indicate that Candida auris can be detected at levels below 10 CFU/mL in blood. “CO” indicates cutoff.

TABLE 12
Analytical sensitivity testing of C. auris

| C. auris Titer | | | | | |
|---|---|---|---|---|---|
| (CFU/mL) | | C. auris | C. haemulonii | C. lusitaniae | Candida IC |
| 5.3 | Average T2MR (msec) | 1048 | 36 | 34 | 1121 |
| | Std. Dev. | 87 | 2 | 1 | 156 |
| | CV | 8% | 6% | 3% | 14% |
| | Hit rate (CO: 65, IC: 85) | 4/4 | 0/4 | 0/4 | 4/4 |
| 10.7 | Average T2MR (msec) | 994 | 36 | 34 | 751 |
| | Std. Dev. | 53 | 1 | 2 | 252 |
| | CV | 5% | 3% | 5% | 34% |
| | Hit rate (CO: 65, IC: 85) | 7/7 | 0/7 | 0/7 | 7/7 |
| 21.3 | Average T2MR (msec) | 1021 | 35 | 35 | 850 |
| | Std. Dev. | 59 | 2 | 2 | 337 |
| | CV | 6% | 6% | 5% | 40% |
| | Hit rate (CO: 65, IC: 85) | 10/10 | 0/10 | 0/10 | 10/10 |

### Example 6: Detection of Candida auris in Buffer Diluents for Swabs

The ability of the assay to detect Candida auris in common buffer diluents for swabs (e.g., axilla/groin swabs) was evaluated using the manual assay and the T2DX® instrument. Phospho-buffered saline/TWEEN® (PBST) and Amies buffer (also referred to as Amies transport buffer or Amies medium) with or without additives (lysis buffer, EDTA, or PBST added in Amies) were tested by the manual assay or on the T2DX® instrument essentially as described in Example 1.

TABLE 13
Testing PBST and Amies buffers with and without Lysis Buffer
in the Manual Assay Results Positive/No. of samples run

| Species & Titer/ | | PBST + | | Amies + |
|---|---|---|---|---|
| Internal Control | PBST | Lysis | Amies | Lysis Buffer |
| C. auris @ 20-21 CFU/mL | 10/10 | 10/10 | 10/10 | 10/10 |
| CIC | 10/10 | 10/10 | 3/10 | 6/10 |

The plating titer for C. auris was 21.2 and 20.3 CFU/mL for Amies and PBST, respectively. As shown in Table 14, 100% detection was observed for C. auris in both buffers using the manual assay. Candida IC (“CIC”) performed well with the PBST buffer (Table 13). The CIC did not amplify as reliably in the Amies buffer, with or without Lysis buffer (Table 13).

TABLE 14
Testing PBST and Amies Buffers with and without
EDTA on the T2DX ® Instrument

| | | | Detection Channel | |
|---|---|---|---|---|
| | | | (Positive/Total # Run) | |
| Sample | Vacutainer | Buffer | C. auris | CIC |
| Negative | No Add | PBST | 0/7 | 7/7 |
| Negative | No Add | Amies | 0/7 | 1/7 |
| C. auris (6.4 CFU/mL) | No Add | PBST | 7/7 | 7/7 |
| C. auris (6.4 CFU/mL) | K₂EDTA | Amies | 7/7 | 4/7 |

Next, detection of C. auris in common buffer diluents for swabs was evaluated on the T2DX® instrument. In vacutainers that did not contain additives, the CIC was detected in 7/7 PBST samples tested on the T2DX® instrument but only in 1/7 Amies samples (Table 14). To improve the performance of the assay with the Amies buffer, the next set of Amies samples were prepared in EDTA vacutainers. Amies transport medium contains both magnesium and calcium salts, an increase in the concentration of divalent cations could potentially inhibit amplification. This effect could be attenuated by the addition of a chelating agent such as EDTA. This worked well for the CIC (7/7) but C. auris had dropouts in Amies medium (4/7) (Table 14). Similar results were obtained using Amies buffer sourced from BD and with an in-house formulation. The next step was to test the addition of 10% of 10×PBST to the Amies buffer (volume/volume for a final concentration of 1×PBST) to evaluate whether the addition of PBST to Amies buffer improved assay performance. By this time, OIC was determined to work well in a multiplex assay with the short Candida primer mix (see Example 2) and was used in all subsequent studies. As shown in Table 15, 100% detection was observed with C. auris spiked samples in Amies containing 10% (v/v) of 10×PBST. Based on these results, further experiments were carried out in this formulation.

TABLE 15
Testing of C. auris Spiked in Amies Buffer + 10% 10x PBST

| Sample | | | | |
|---|---|---|---|---|
| C. auris @ 4.9 CFU/mL in | Detection Channel | | | |
| Amies + 10x PBST (10% v/v) | C. auris | C. duobushaemulonii | C. haemulonii | OIC |
| Average T2 Signal (msec) | 1322 | 31 | 32 | 631 |
| Std. Dev. | 122 | 1 | 1 | 71 |
| % CV | 9% | 3% | 5% | 11% |
| Hit Rate (CO: 65 ms, | 7/7 | 0/7 | 0/7 | 7/7 |
| IC: 85 ms) | | | | |

Next, the performance of the assay for detection of the four known clades of C. auris in PBST and Amies buffer was evaluated. For PBST, 7/7 samples were detected for CAU-01 and CAU-04 (Table 16). CAU-02 and CAU-05 were detected in 6/7 and 3/7 samples, respectively (Table 16). These two isolates were also spiked at the lowest concentrations, and therefore, were repeated at higher spike concentrations. 7/7 samples were detected for CAU-02 at 23.9 CFU/mL and 6/6 valid samples were detected for CAU-05 at 3.7 CFU/mL (Table 16). Candida auris at 2.8 CFU/mL was detected in 5/7 in-house Amies samples and in 5/5 BD Amies samples with 10% 10×PBST (Table 16).

TABLE 16
Testing Different C. auris Clades Spiked in PBST or Amies Buffers

| PBST | | C. auris | C. duobushaemulonii | C. haemulonii | OIC |
|---|---|---|---|---|---|
| CAU-01 8.0 | Average (Avg) T2 Signal | 1147 | 31 | 3 | 474 |
| CFU/mL | (msec) | | | | |
| Clade II | Std. Dev. | 78 | 3 | 1 | 171 |
| | % CV | 7% | 8% | 3% | 36% |
| | Hit rate (CO: 65, IC: 85) | 7/7 | 0/7 | 0/7 | 7/7 |
| CAU-02 3.5 | Avg T2 Signal (msec) | 1070 | 31 | 32 | 521 |
| CFU/mL | Std. Dev. | 418 | 3 | 1 | 184 |
| Clade I | % CV | 39% | 9% | 4% | 35% |
| | Hit rate (CO: 65, IC: 85) | 6/7 | 0/7 | 0/7 | 6/7 |
| CAU-02 23.9 | Avg T2 Signal (msec) | 1087 | 30 | 30 | 484 |
| CFU/mL in | Std. Dev. | 96 | 1 | 2 | 191 |
| PBST Clade I | % CV | 9% | 5% | 5% | 40% |
| | Hit Rate (CO: 65, IC: 85) | 7/7 | 0/7 | 0/7 | 6/7 |
| CAU-04 6.4 | Avg T2 Signal (msec) | 1029 | 34 | 3 | 1333 |
| CFU/mL | Std. Dev. | 38 | 1 | 2 | 71 |
| Clade III | % CV | 4% | 4% | 5% | 5% |
| | Hit rate | 7/7 | 0/7 | 0/7 | 7/7 |
| CAU-05 0.2 | Avg T2 Signal (msec) | 1101 | 30 | 32 | 480 |
| CFU/mL | Std. Dev. | 429 | 1 | | 216 |
| Clade IV | % CV | 39% | 4% | 3% | 45% |
| | Hit rate (CO: 65, IC: 85) | 3/7 | 0/7 | 0/7 | 7/7 |
| CAU-05 3.7 | Avg T2 Signal (msec) | 1099 | 30 | 31 | 498 |
| CFU/mL in | Std. Dev. | 480 | 1 | 1 | 240 |
| PBST Clade | % CV | 44% | 2% | 4% | 48% |
| IV | Hit Rate (CO: 65, IC: 85) | 6/7 | 0/7 | 0/7 | 6/7 |

| Amies | | C. auris | C. duobushaemulonii | C. haemulonii | OIC |
|---|---|---|---|---|---|
| CAU-04 2.8 | Avg T2 Signal (msec) | 1112 | 32 | 39 | 474 |
| CFU/mL in | Std. Dev. | 285 | 1 | 4 | 186 |
| BD Amies | %CV | 26% | 4% | 11% | 39% |
| Clade III | Hit Rate (CO: 65, IC: 85) | 5/5 | 0/5 | 0/5 | 5/5 |
| CAU-04 2.8 | Avg T2 Signal (msec) | 940 | 32 | 33 | 559 |
| CFU/mL in | Std. Dev. | 619 | 1 | 3 | 112 |
| T2Amies | % CV | 66% | 5% | 9% | 20% |
| Clade III | Hit Rate (CO: 65, IC: 85) | 5/7 | 0/7 | 0/7 | 7/7 |

The analytical sensitivity for detection of C. auris in PBST or Amies buffer with 10% PBST was evaluated on the T2DX® instrument. As shown in Table 18, 100% detection was observed with samples spiked in Amies containing 10% of 10×PBST and samples in 1×PBST (Table 17). In conclusion, these data demonstrate that PBST and Amies+10% of 10×PBST can be used as diluents to detect C. auris at low titers (<10 CFU/mL) on the T2Dx. The primers (i.e., SEQ ID NO: 1 and 3) can detect all four clades of the C. auris. These buffer diluents can be used to detect Candida species such as C. auris in samples from swabs (e.g., axilla/groin swabs).

TABLE 17
Detection of C. auris Spikes at <10 CFU/mL in PBST and
Amies Buffers on the T2DX ® Instrument

| Sample Details | | C. auris | C. duobushaemulonii | C. haemulonii | OIC |
|---|---|---|---|---|---|
| C. auris | Average T2 Signal | 1122 | 36 | 38 | 461 |
| 6.9 CFU/mL in | (msec) | | | | |
| 1x PBST in | Hit Rate | 9/9 | 0/9 | 0/9 | 9/9 |
| EDTA | (Positive/Total # | | | | |
| Vacutainers | Samples) | | | | |
| C. auris | Average T2 Signal | 1,205 | 38 | 36 | 536 |
| 9.2 CFU/mL in | (msec) | | | | |
| Amies with | Hit Rate | 10/10 | 0/9 | 0/9 | 10/10 |
| 10% 10x PBST | (Positive/Total # | | | | |
| in EDTA | Samples) | | | | |
| Vacutainers | | | | | |

### Example 7: Analytical Sensitivity of T2Cauris Assay for Candida Duobushaemulonii

A panel containing C. duobushaemulonii (C. db), C. auris (C. au), and C. haemulonii (C. h) was developed. The T2DX® instrument was evaluated with C. duobushaemulonii particles for detection of C. duobushaemulonii low titer spikes (3 CFU/mL) in blood samples. Table 18 shows the full length (FL) sequences of the panel probe sequences, which include 5′ or 3′ 9 bp poly-T linker sequences that allow conjugation to magnetic particles at either the 5′ or 3′ end. Table 18 also shows the specific portions of the probes which do not include the linker sequences. The result was 100% detection (7/7) for C. duobushaemulonii at 3 CFU/mL with no cross reactivity, confirming that the particles are species specific (Table 19). These results demonstrate that the assay detects C. duobushaemulonii in blood samples at titer levels below 5 CFU/mL.

TABLE 18
T2Cauris Panel Probe Sequences

| Probes | Sequence |
|---|---|
| C. au 5′ FL | TTT TTT TTT CTA CCT GAT TTG AGG CGA CAA CAA AAC |
| | (SEQ ID NO: 49) |
| C. au 3′ FL | CCG CGA AGA TTG GTG AGA AGA CAT TTT TTT TTT |
| | (SEQ ID NO: 50) |
| C. db 5′ FL | TTT TTT TTT CGT AGA CTT CGC TGC GGA T (SEQ ID NO: 51) |
| C. db 3′ FL | CTG GGC GGT GAG AAG AAA TCT TTT TTT TT (SEQ ID NO: 52) |
| C. h 5′ FL | TTT TTT TTT GTC CTA CCT GAT TTG AGG GGA AAA AGC |
| | (SEQ ID NO: 53) |
| C. h 3′ FL | AAC AAA TCC ACC AAC GGT GAG AAG ATA TTT TTT TTT T |
| | (SEQ ID NO: 54) |
| OIC 5′ FL | TTT TTT TTT CGA TGG TTC ACG GGA TTC TGC AAT TC |
| | (SEQ ID NO: 55) |
| OIC 3′ FL | GAG ACG TTT TGG ATA CAT GTG AAA GAA GGC TTT TTT TTT |
| | (SEQ ID NO: 56) |
| C. au 5′ | CTA CCT GAT TTG AGG CGA CAA CAA AAC (SEQ ID NO: 4) |
| C. au 3′ | CCG CGA AGA TTG GTG AGA AGA CAT (SEQ ID NO: 5) |
| C. db 5′ | CGT AGA CTT CGC TGC GGA T (SEQ ID NO: 48) |
| C. db 3′ | CTG GGC GGT GAG AAG AAA TC (SEQ ID NO: 29) |
| C. h 5′ | GTC CTA CCT GAT TTG AGG GGA AAA AGC (SEQ ID NO: 8) |
| C. h 3′ | AAC AAA TCC ACC AAC GGT GAG AAG ATA T (SEQ ID NO: 9) |
| OIC 5′ | CGA TGG TTC ACG GGA TTC TGC AAT TC (SEQ ID NO: 40) |
| OIC 3′ | GAG ACG TTT TGG ATA CAT GTG AAA GAA GGC (SEQ ID NO: 39) |

TABLE 19
Testing of C. duobushaemulonii particles

| C. duobushaemulonii @ 3 | Detection Channel | | | |
|---|---|---|---|---|
| CFU/mL in 1X PBST | C. auris | C. duobushaemulonii | C. haemulonii | OIC |
| Average T2 Signal (msec) | 45 | 929 | 45 | 432 |
| Std. Dev. | 3 | 243 | 7 | 148 |
| % CV | 7% | 26% | 14% | 34% |
| Positive Hit Rate (Cut off 65 ms, | 0/7 | 7/7 | 0/7 | 7/7 |
| IC 85 ms) | | | | |

### Example 8: PCR Parameter Optimization

Conditions for amplifying C. auris, C. duobushaemulonii, C. haemulonii, and the OIC were evaluated. Genomic DNA (gDNA) was isolated by using the QUICK-DNA™ Fungal/Bacterial Miniprep Kit (Zymo, D6005) following the manufacturer's instructions. The gDNA was quantified by spectroscopy. 10 and 50 copies of gDNA were amplified on the Eppendorf MASTERCYCLER® with annealing temperatures of 58° C. or 61° C. for 40, 42, 44 and 46 cycles, with N=8 per condition. All conditions yielded high T2 values and low variation (FIGS. 3A-3D and 3E-3H, respectively). These data demonstrate the robustness of the assay to variations in annealing temperature. For subsequent experiments, amplification conditions of 61° C. for 42 cycles were selected for manual assay testing and optimization.

### Example 9: Evaluation of Effect of Amplification Inhibitors on Assay

The experiments in this Example evaluated the effect of the presence of amplification inhibitors in buffer and blood lysate on amplification of OIC and Candida target nucleic acids. If the internal control does not amplify, and all channels are negative, the sample is flagged as an IC invalid. The present experiments were performed to tune the IC to ensure that in the presence of common inhibitors, amplification of the IC is inhibited before the target nucleic acids. If the reverse were to occur, the assay could result in a false negative. The target IC concentration in these experiments was 250-400 copies of IC per reaction, with a target of 300 copies/reaction.

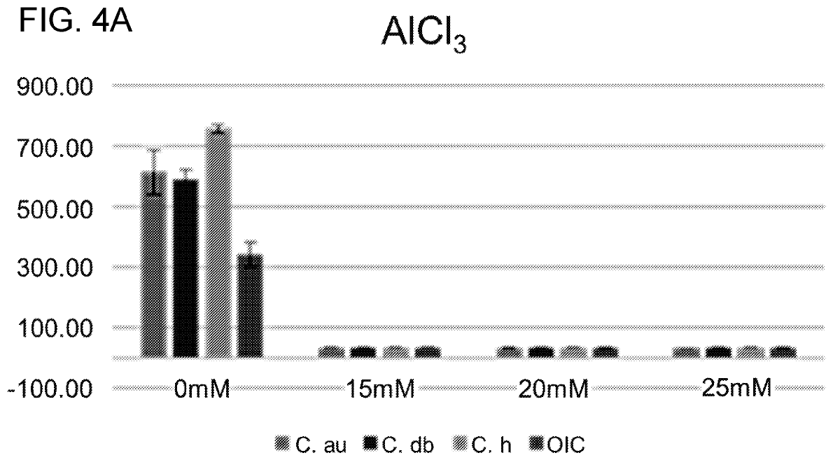

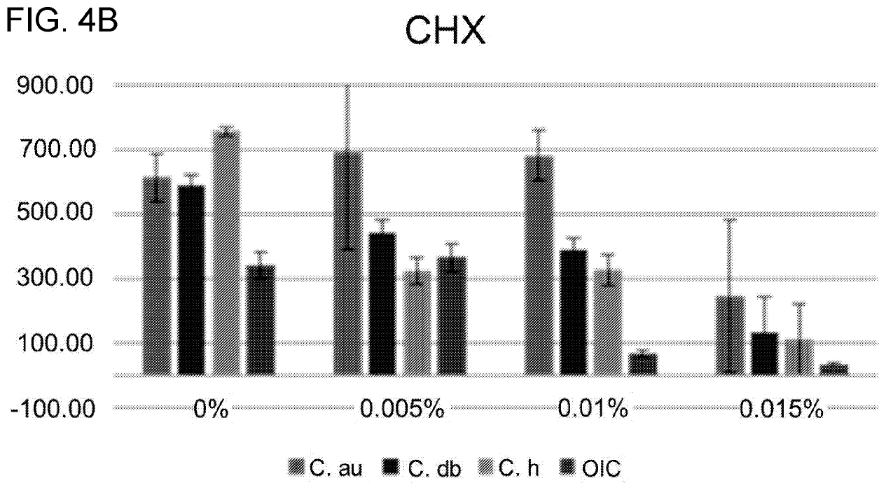

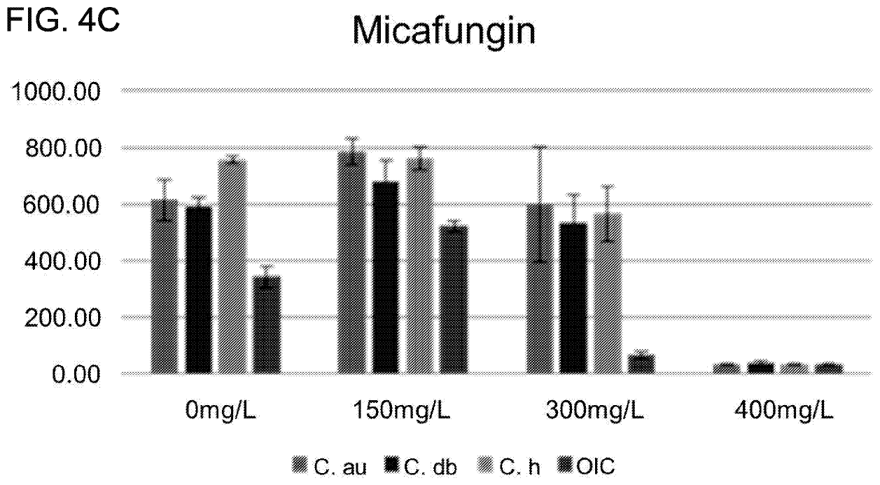

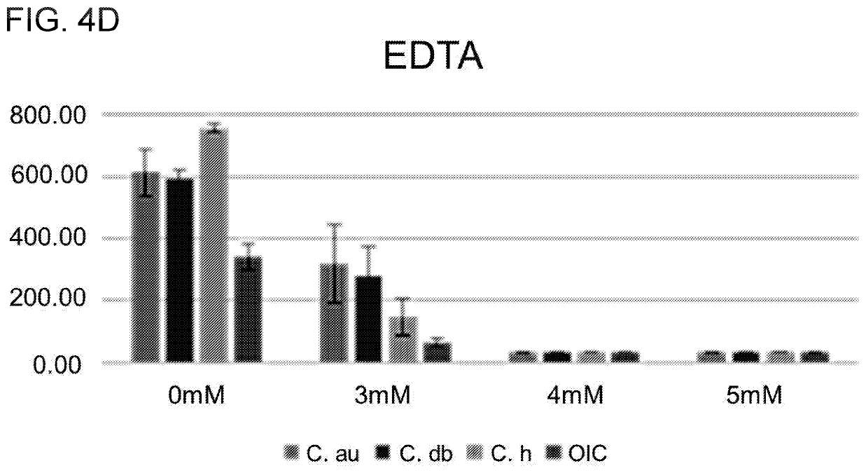

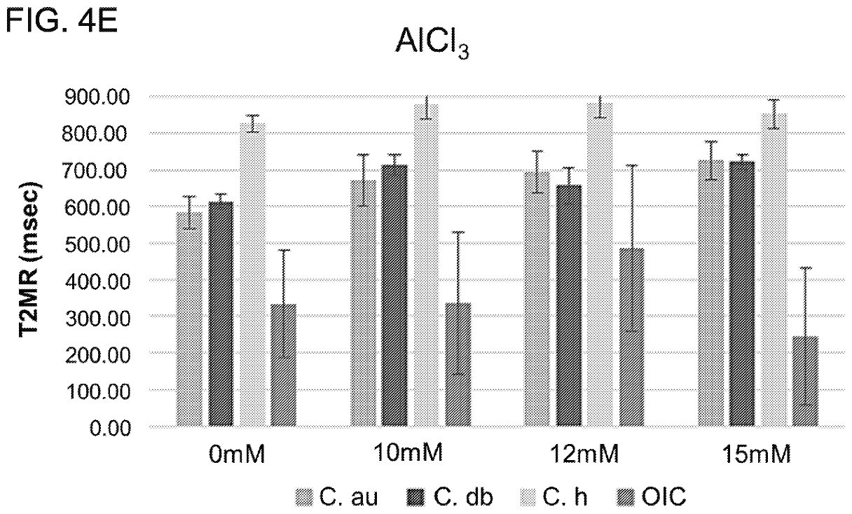

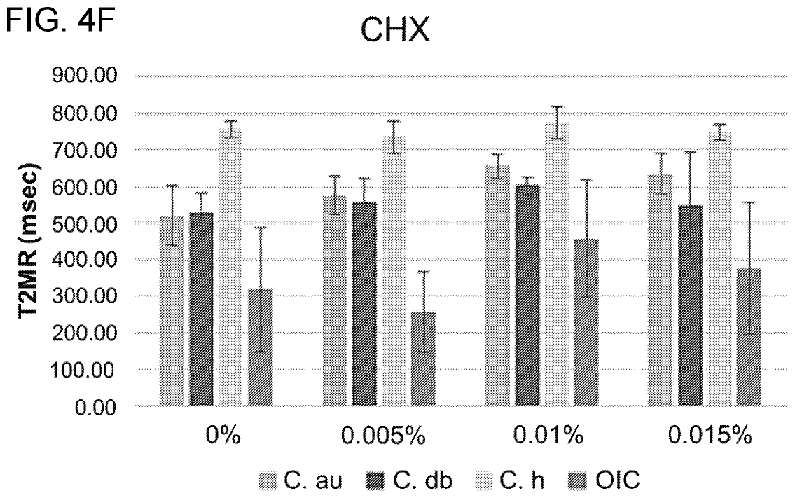

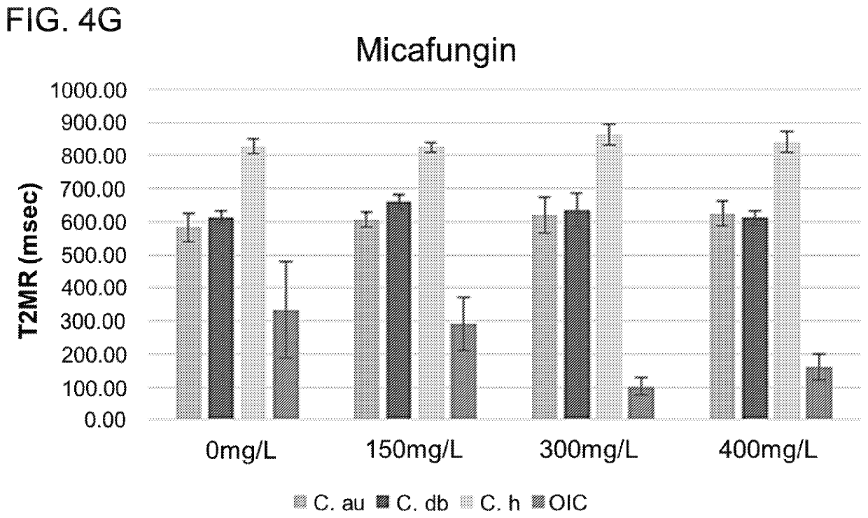

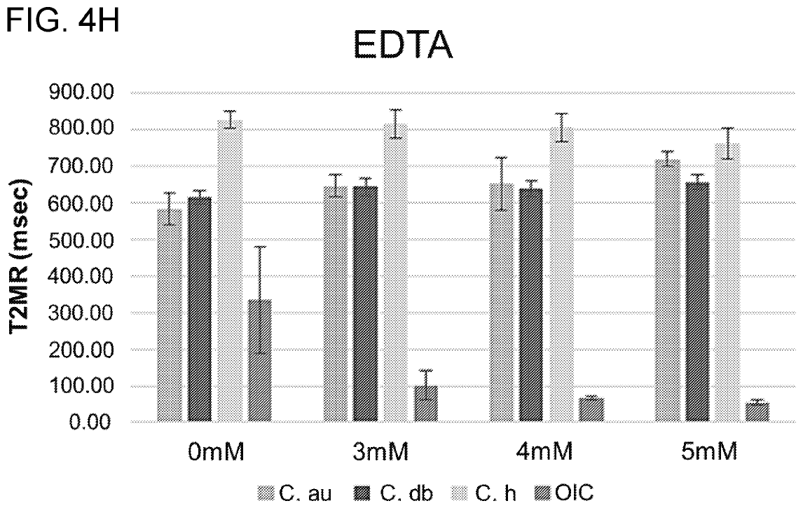

For buffer, 10 copies of gDNA per reaction were triple spiked in Tris EDTA (TE) buffer with or without inhibitors. The inhibitors that were tested were (1) aluminum chloride (AlCl₃), a major component of deodorant; (2) chlorhexidine (CHX), commonly used topical antiseptic and disinfectant; (3) micafungin, an antifungal compound; and (4) EDTA, a known amplification inhibitor. The concentrations that were tested for each inhibitor (N=6 per condition) are outlined in Table 20. The amplification was carried out in the Eppendorf MASTERCYCLER®. OIC amplification was inhibited before the targets in the presence of increasing concentrations of chlorohexidine, micafungin, or EDTA (FIGS. 4B-4D). OIC amplification was inhibited concurrently with the targets in presence of aluminum chloride (FIG. 4A).

TABLE 20
Concentrations of Inhibitors used

| | |
|---|---|
| AlCl₃ (mM) | 0, 15, 20, 25 |
| CHX (% (w/v)) | 0, 0.005, 0.01,0.015 |
| MF (mg/L) | 0, 150, 300, 400 |
| EDTA (mM) | 0, 3, 4, 5 |

For blood lysate, 10 copies of gDNA per reaction were triple spiked in blood lysate with or without inhibitors. The inhibitors that were tested were (1) aluminum chloride (AlCl₃), a major component of deodorant; (2) chlorhexidine (CHX), commonly used topical antiseptic and disinfectant; (3) micafungin, an antifungal compound; and (4) EDTA, a known amplification inhibitor. The concentrations that were tested for each inhibitor (N=6 per condition) are outlined in Table 21. The amplification was carried out in the Eppendorf MASTERCYCLER®. Since the OIC failed before the targets in three of the four compounds tested for both buffer and blood lysate, the levels of OIC were not changed (FIGS. 4E-4H).

TABLE 21
Concentrations of Inhibitors used

| | |
|---|---|
| AlCl₃ (mM) | 0, 10, 12, 15 |
| CHX (% (w/v)) | 0, 0.005, 0.01, 0.015 |
| MF (mg/L) | 0, 150, 300, 400 |
| EDTA (mM) | 0, 3, 4, 5 |

### Example 10: Reagent Guard Banding

The experiments in this Example evaluated the tolerance of the T2Cauris amplification reaction to the variation of key reagents in buffer or blood lysate.

Methods

A 1/16^{th} fraction, Resolution IV factorial was designed to study the key reagents in the T2Cauris Panel as listed below in Table 22. The reagents that were tested were C. auris Forward (Candida Short from Example 2) and Reverse primers, OIC Forward and Reverse primers, MgCl₂, 5× buffer dNTPs, DNA polymerase enzyme at ±10% variation in concentration, and ±0.25 pH units. These were tested by triple spiking 10 copies each of C. auris, C. duobushaemulonii, and C. haemulonii DNA in OIC final bulk. The same test was also carried out in blood lysate. N=8 per mix. The amplification was carried out in the Eppendorf MASTERCYCLER®.

TABLE 22
Factorial Design to Study Variation in
the Reagents for the T2Cauris Panel

| | | | | | | | | En- |
|---|---|---|---|---|---|---|---|---|
| Mix | C. au F | C. au R | OIC F | OIC R | MgCl₂ | pH | dNTP | zyme |
| 1 | −1 | −1 | −1 | 1 | 1 | 1 | −1 | 1 |
| 2 | −1 | −1 | −1 | −1 | −1 | −1 | −1 | −1 |
| 3 | 1 | −1 | −1 | −1 | −1 | 1 | 1 | 1 |
| 4 | −1 | −1 | 1 | −1 | 1 | 1 | 1 | −1 |
| 5 | −1 | 1 | 1 | 1 | 1 | −1 | −1 | −1 |
| 6 | 1 | 1 | −1 | −1 | 1 | 1 | −1 | −1 |
| 7 | 1 | −1 | 1 | −1 | 1 | −1 | −1 | 1 |
| 8 | 1 | −1 | −1 | 1 | 1 | −1 | 1 | −1 |
| 9 | 0 | 0 | 0 | 0 | 0 | 0 | 0 | 0 |
| 10 | 1 | 1 | 1 | 1 | 1 | 1 | 1 | 1 |
| 11 | −1 | 1 | 1 | −1 | −1 | 1 | −1 | 1 |
| 12 | −1 | 1 | −1 | 1 | −1 | 1 | 1 | −1 |
| 13 | 1 | 1 | 1 | −1 | −1 | −1 | 1 | −1 |
| 14 | −1 | −1 | 1 | 1 | −1 | −1 | 1 | 1 |
| 15 | 1 | 1 | −1 | 1 | −1 | −1 | −1 | 1 |
| 16 | 1 | −1 | 1 | 1 | −1 | 1 | −1 | −1 |
| 17 | −1 | 1 | −1 | −1 | 1 | −1 | 1 | 1 |

Buffer

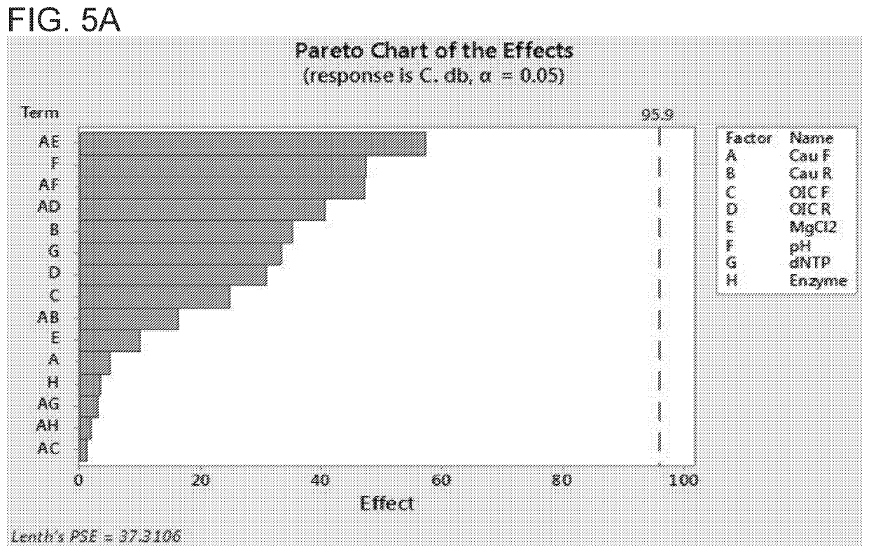

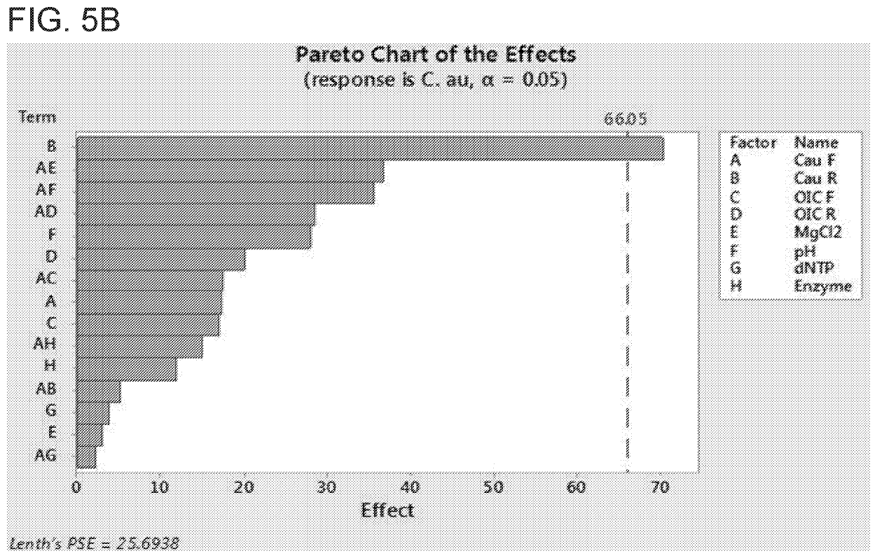

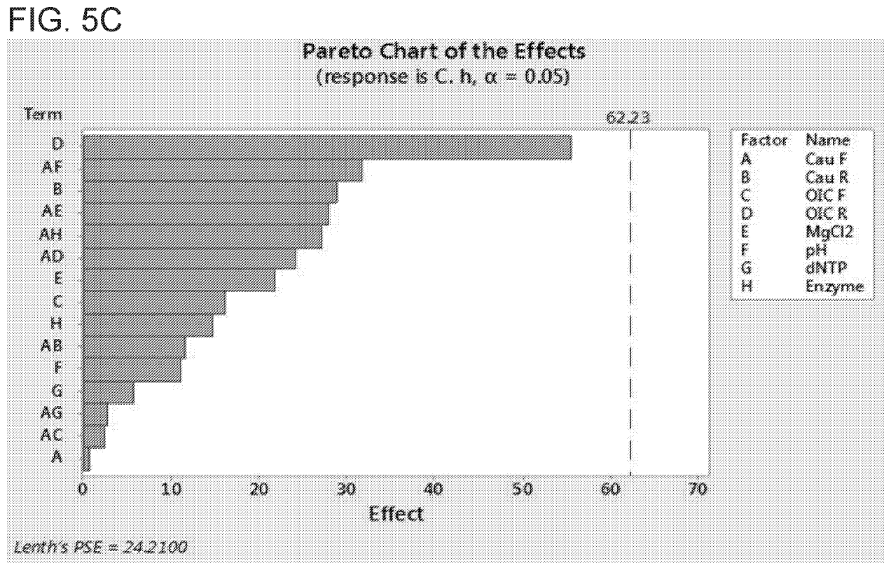

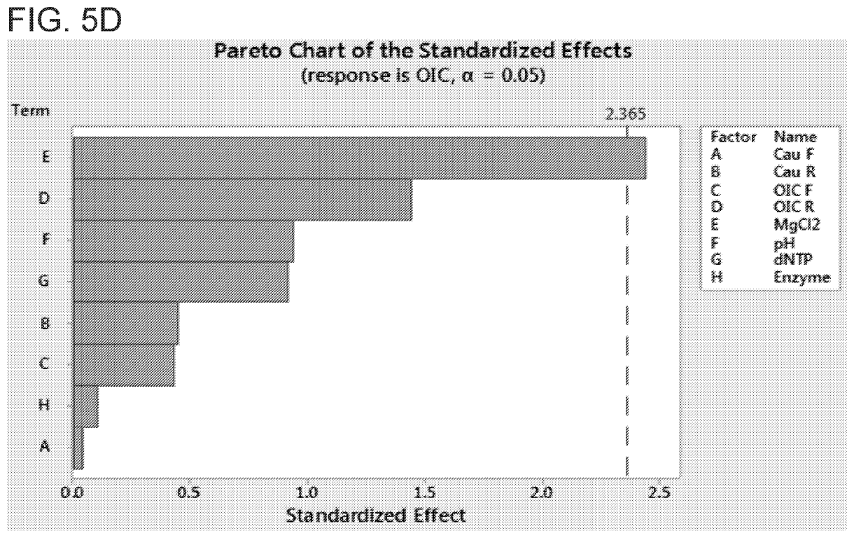

Analysis of the design of experiments (DOE) data identified the C. auris Reverse primer as a significant factor on the average T2MR signal of C. auris (FIGS. 5A-5D). While the C. auris Reverse primer was identified as a significant factor, no dropouts or large decreases in T2MR signal were observed. Therefore, the observed change in T2MR signal caused by the reverse primer was determined not to be a risk. MgCl₂ was identified as a significant factor on the average T2MR signal of OIC, with an increase of MgCl₂ causing a statistically significant decrease in the OIC T2MR signal. Following the observation that a 10% increase in the concentration of MgCl₂ leads to a decrease in T2MR signal, a follow up experiment was designed titrating at ±5% [MgCl₂] (FIGS. 6A-6D). In this experiment, there was no statistical difference between the average T2MR signals of the nominal compared to the tested reaction buffers.

Blood Lysate

Analysis of the DOE data identified pH as a significant factor on the average T2MR signal of both OIC and C. duobushaemulonii (FIGS. 7A-7D). For C. duobushaemulonii, the average T2MR signal was >500 msec for all mixes with no dropouts observed, indicating that a variation of ±0.25 pH units is tolerated. For OIC, in mixes that contained high pH and high dNTPs, dropouts and significantly lower signals were observed, therefore, these components were directly tested with smaller variations in the tested factor. Testing was carried out as the original DOE but with +0.125 pH units and +5% dNTPs (FIG. 8). No dropouts were observed in any conditions. OIC signal was observed to be more sensitive to pH than target species.

Conclusions

These data indicate that the T2Cauris assay is robust, with tolerances of at least ±10% of the nominal concentration for the C. auris and OIC primers and DNA polymerase enzyme; at least ±5% for dNTP and MgCl₂; and at least ±0.125 units for pH in both blood and buffer.

### Example 11: Equivalence of Single Versus Multiple Spikes

The equivalence in T2MR signals of single species spike versus triple species spike in PBST and Amies buffer with 10% (v/v) 10×PBST was evaluated. Single and triple spikes in PBST and Amies+10% (v/v) 10×PBST at 10 CFU/mL for each condition were processed by manual assay method essentially as described in Example 1 with N=6 per condition. One C. duobushaemulonii false negative was observed when single spiked in Amies buffer, and there was relatively high variability in the T2 signals in Amies buffer (FIG. 9).

Overall, mean T2 signals for single versus triple spikes were equivalent for all channels irrespective of buffer (PBST and Amies+10% 10×PBST) (P>0.05 for single spike>multi spike equivalence test for all channels). Candida cells spiked in PBST and Amies were not equivalent, but further optimization in the growth method and spiking as described in Example 12 led to equivalence being demonstrated between the buffers.

### Example 12: Spiking from Candida auris Cell Bullets

Spiking samples from Candida auris cell bullets rather than fresh cell culture was evaluated. This approach saves 2-3 days of culture time and gives higher counting accuracy. Triple species spikes were tested in PBST and in Amies+10% (v/v) 10×PBST at 10 CFU/mL for each species by manual assay method essentially as described in Example 1 with N=8 per condition. No false negatives were observed. High T2 signals and low variation were obtained for all species and OIC in PBST and Amies buffers (FIG. 10). The 95% Confidence Intervals for the mean T2 signals overlapped between the species irrespective of the spike buffer. The spiking from the bullets eliminated the differences in PBST and Amies buffer spikes that was seen with the previous growth conditions from Example 11. This growth method was adopted for subsequent studies.

### Example 13: Evaluation of Potential Swab Inhibitors and a New Swab Matrix

The effect of potential interfering substances on the T2Cauris assay was evaluated. Since patient swabs are typically axial, deodorant could be present in samples. Cary Blair is a transport medium used to transport samples and activated charcoal is typically present in swab kits. For these experiments, the swab included with the Cary Blair Gel was placed into the gel. The swab and gel were mixed, and the swab was removed with a small amount of semi-solid gel and placed into the vacutainer. The swab was swished in the vacutainer 10 times. The gel and swab were then discarded. Deodorant or activated charcoal swabs were introduced to 50 CFU/mL C. auris spikes and the samples were run on the T2DX® instrument. The charcoal and deodorant concentrations are estimated to be about 0.1 to about 0.5 g/mL of sample. As shown in Table 23, the assay resulted in 100% species specific detection in presence of potential interfering substances and in the Cary Blair media. Therefore, there is no evidence that the assay is inhibited in the presence of potential interfering substances at the quantities tested.

TABLE 23
Testing the impact of common inhibitors in the swab media on C. auris detection

| | | | Detection Channel | | |
|---|---|---|---|---|---|
| | Concentration | | (Positive/Total # samples) | | |
| Species | (CFU/mL) | Matrix | C. auris | C. duobushaemulonii | C. haemulonii |
| Candida auris | 50 | Activated | 4/4 | 0/4 | 0/4 |
| | | Charcoal + | | | |
| | | PBST | | | |
| Candida auris | 50 | Deodorant | 4/4 | 0/4 | 0/4 |
| | | Swab + PBST | | | |
| Candida auris | 50 | Cary Blair | 4/4 | 0/4 | 0/4 |
| | | Media | | | |

### Example 14: C. auris Spiked Sample Stability

The stability of sample spikes prepared with C. auris, C. duobushaemulonii, and C. haemulonii bullets and stored at 2-8° C. was evaluated. Triple spiked samples prepared in either 1×PBST or Amies+10% 10×PBST and stored in K₂EDTA vacutainers at 2-8° C. were tested on Day 0, 1, 3, 7 and 8, and N=6 samples were processed per time point per buffer by the manual assay method essentially as described in Example 1. An analysis of T2Cauris data from PBST and Amies triple spiked with each of the target species (C. auris, C. duobushaemulonii, or C. haemulonii) at 10 CFU/mL tested by the manual assay was performed to determine the number of replicates (N) required for the execution of sample stability study (Table 24). According to these data, the mean T2 value minus 2 standard deviations falls within the an appropriate maximum allowable drift, T=0 2SD, range. Appropriate N with sufficient power (80%) to detect significant changes (dmax) with sufficient confidence (95%) was determined using the one-sided test equation in calculating the testing requirements of measurand drift based on linear regression. The minimum number of replicates necessary to demonstrate 80% power with a significance of 0.05 was five (N=5) for C. auris, three (N=3) for C. duobushaemulonii and four (n=4) for C. haemulonii. We ran six (N=6) based on the largest number of required replicates calculated among all channels. The sample size justification, as displayed in Table 24 below, determined that the number of repeats required per time point was one (1), when testing N=6 replicates over a total of five (5) time points for a linear regression-based stability study.

TABLE 24
Sample size calculation
C. auris Samples

| Metric | C. au | C. db | C. h |
|---|---|---|---|
| N | 28 | 28 | 28 |
| Concentration (CFU/mL) | 10 | 10 | 10 |
| Mean T2 Signal (msec) | 709 | 687 | 782 |
| Test method repeatability (SD) | 148 | 108 | 138 |
| % CV | 21 | 16 | 18 |
| 2SD | 295 | 217 | 277 |
| Mean - 2SD | 414 | 471 | 505 |
| N (Number of replicates/time point/Matrix) | 6 | 6 | 6 |
| P (Number of time points) | 5 | 5 | 5 |
| R (Number of repeats/time point) | 1 | 1 | 1 |
| α | 0.05 | 0.05 | 0.05 |
| Power | 0.80 | 0.80 | 0.80 |
| Total Number of tests needed | 30 | 30 | 30 |

| | |
|---|---|
| Time points | 0, 1, 3, 7, 8 days |

The mean T2 signals for all species for all time points were above the lower cutoff limit for the respective species. These data demonstrate that C. auris samples spiked from bullets into PBST or Amies buffer as low as 10 CFU/mL are stable at 2-8° C. for up to 7 days.

### Example 15: Four Member T2Cauris Panel

A T2 Magnetic Resonance (T2MR®) platform for highly sensitive, rapid species level identification of C. auris, C. haemulonii, C. duobushaemulonii or C. lusitaniae in whole blood samples or from common patient and environmental swab matrices was developed.

Methods

Cell Preparation for Laboratory Testing

All isolates used were from the CDC-FDA AR Bank. C. auris, C. lusitaniae, C. duobushaemulonii and C. haemulonii cultured cells were spiked in K₂EDTA anticoagulated blood from healthy human donors or into Amies medium or a PBS Tween® buffer (PBST). Candida cells were grown overnight in Yeast-Peptone-Dextrose media at 30° C. and cell concentration determined using an automated cell counter. From this stock, the culture was diluted to a target concentration to allow for a 1:100 addition to either healthy K₂EDTA-treated human whole blood, PBST, or Amies medium to achieve a final spike concentration. All spike concentrations were confirmed by plating of the cell solution used for spiking on YPD agar medium.

T2MR Candida auris Panel

Spiked blood or swab eluent buffer samples were processed on a T2DX® Instrument, which automates the following steps: chemical lysis of blood cells (if required), concentration of target cells through centrifugation, release of target cell DNA through mechanical lysis, and amplification of target DNA (FIG. 4). The T2Dx detects and identifies the presence of each individual species by hybridizing the amplicon with DNA probe conjugated superparamagnetic particles. The particles cluster only in the presence of the species they are directed against, and the resulting clustering is identified by the T2 relaxation signal. Target-specific DNA induced clustering results in 30× higher T2MR signal versus dispersed particles allowing for sensitive

detection and identification of Candida species direct from whole blood.

Results

Highly Sensitive DNA Detection by T2MR

T2MR detection is highly sensitive to small amounts of target DNA that have been amplified in the presence of background human DNA. Titrations of oligomers representing the Candida target sequences indicate that concentrations as low as 1 E+10 copies per reaction can be reliably detected with T2MR (FIG. 11).

C. auris cells were spiked into K₂EDTA anticoagulated whole blood at decreasing concentrations. 100% detection on the T2Dx Instrument was observed for samples spiked at 5 CFU/mL direct from whole blood without blood culture (Table 12). T2MR signals of samples spiked with target were approximately 30 times higher than samples with no target present, no cross reactivity was observed between C. auris, C. haemulonii, and C. lusitaniae.

Differentiation of Individual Species

The panel is designed in a manner such that the detection of the targets are species specific. The Candida Forward Short (SEQ ID NO: 1) and Candida Reverse (SEQ ID NO: 3) primers are used for amplification. Probes as described in Table 18 were used for detection of C. auris, C. duobushaemulonii and C. haemulonii. Probes as described in Table 1 were used for detection of C. lusitaniae. Particles can be functionalized with probes to detect a single target or dual targets in a detection channel. No cross-reactivity was observed when particles directed toward one or two species are tested against other Candida species, even in high concentration spikes (Table 10). Clades I, II and III of Candida auris were tested and shown not to cross react. The panel is designed in a manner such that amplification and detection of the targets are species specific. No cross-reactivity is observed when particles directed toward one species are tested against the other two species (Table 10).

High Sensitivity Detection in Common Swab Eluents

The flexible T2DX® platform allows for high sensitivity detection of Candida species in common swab eluents such as Amies medium and PBS Tween buffer. Rapid environmental and patient sampling will enable a timelier implementation of prevention and infection control measures and potentially help prevent the spread of infection within affected healthcare facilities. Sensitivities of below <10 CFU/mL will allow for the pooling of multiple samples and screening of patients and surfaces (Table 25).

TABLE 25
High Sensitivity Detection in Swab Eluents

| | Concentration | | T2MR Detection Channel (# Positive/#Run) | | |
|---|---|---|---|---|---|
| Target | (CFU/mL) | Matrix | C. auris | C. haemulonii | C. duobushaemulonii |
| C. auris | 5-9 | Amies | 16/16 | 0/16 | 0/16 |
| C. auris | 6-7 | PBST | 17/17 | 0/17 | 0/17 |
| C. duobushaemulonii | 4 | Amies | 0/7 | 0/7 | 7/7 |
| C. duobushaemulonii | 3 | PBST | 0/7 | 0/7 | 7/7 |
| C. haemulonii | 7 | Amies | 0/6 | 6/6 | 0/6 |
| C. haemulonii | 7 | PBST | 0/6 | 6/6 | 0/6 |

Broad Species Level Inclusivity

The panel is designed to provide broad coverage of all known C. auris clades (Table 26).

TABLE 26
Detection of C. auris Clades

| | Concentration | | T2MR Detection Channel (# Positive/#Run) | | |
|---|---|---|---|---|---|
| C. auris clades | (CFU/mL) | Matrix | C. auris | C. haemulonii | C. duobushaemulonii |
| Clade I South Asia | 24 | PBST | 7/7 | 0/7 | 0/7 |
| Clade II Asia | 8 | PBST | 7/7 | 0/7 | 0/7 |
| Clade III Africa | 6 | PBST | 7/7 | 0/7 | 0/7 |
| Clade IV South | 4 | PBST | 6/6 | 0/6 | 0/6 |
| America | | | | | |

Testing of Clinical Samples

The panel has been tested in clinical samples from patients with suspicion of candidemia. Agreement was found between blood culture and T2MR using the panel.

### CONCLUSIONS

An assay for the rapid detection of C. auris based on the T2MR technology has been used to detect C. auris and other Candida species directly from whole blood and common swab matrices at concentrations<10 CFU/mL. Testing with clinical samples indicates that this test can be used to identify Candida auris and other species from patient blood samples without requiring blood culture. This rapid and sensitive test enables detection of C. auris and other Candida species in candidemic patients and assist in screening, isolating, and monitoring the spread of this emerging multidrug resistant pathogen.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method for detecting the presence of a Candida species in a biological or environmental sample, wherein the Candida species is Candida auris, the method comprising:
(a) providing a biological or environmental sample;
(b) amplifying a Candida species target nucleic acid in the biological or environmental sample; and
(c) detecting the amplified nucleic acid to determine whether Candida auris is present in the biological or environmental sample,
wherein (i) the presence of Candida auris in the biological or environmental sample is determined within about 5 hours from obtaining the sample or less; (ii) the presence of Candida auris is determined directly from the biological or environmental sample without a prior culturing step; and/or (iii) the Candida auris is present in the biological or environmental sample at a concentration of about 10 cells/mL of biological or environmental sample or less.

2. The method of claim 1, wherein step (c) further comprises detecting the amplified nucleic acid to determine whether Candida lusitaniae is present.

3. The method of claim 1 or 2, wherein step (c) further comprises detecting the amplified nucleic acid to determine whether Candida haemulonii is present.

4. The method of any one of claims 1-3, wherein step (c) further comprises detecting the amplified nucleic acid to determine whether Candida duobushaemulonii is present.

5. The method of any one of claims 1-3, wherein step (c) further comprises detecting the amplified nucleic acid to determine whether Candida pseudohaemulonii is present.

6. The method of any one of claims 1-5, wherein the method detects a concentration of Candida auris of 10 cells/mL of biological or environmental sample or less.

7. The method of any one of claims 1-6, wherein step (a) further comprises lysing Candida cells present in the biological or environmental sample.

8. The method of any one of claims 1-7, wherein the amplified Candida species target nucleic acid is detected by sequencing, optical, fluorescent, mass, density, magnetic, chromatographic, and/or electrochemical measurement.

9. The method of claim 8, wherein the amplified Candida species target nucleic acid is detected by measuring the T₂ relaxation response of the biological or environmental sample or a portion thereof following contacting the biological or environmental sample or the portion thereof with magnetic particles, wherein the magnetic particles have binding moieties on their surfaces, the binding moieties operative to alter the specific aggregation of the magnetic particles in the presence of the amplified Candida species target nucleic acid.

10. A method for detecting the presence of Candida species in a biological or environmental sample, wherein the Candida species is Candida auris, the method comprising:
(a) providing a biological or environmental sample;
(b) preparing an assay sample by contacting a portion of the biological or environmental sample with magnetic particles, wherein the magnetic particles have binding moieties on their surfaces, the binding moieties operative to alter the specific aggregation of the magnetic particles in the presence of an analyte associated with Candida auris;
(c) placing the assay sample in a device, the device comprising a support defining a well for holding the assay sample, and having an RF coil configured to detect a signal produced by exposing the assay sample to a bias magnetic field created using one or more magnets and an RF pulse sequence;
(d) exposing the assay sample to the bias magnetic field and the RF pulse sequence;
(e) following step (d), measuring the signal produced by the assay sample; and
(f) using the results of step (e) to determine Candida auris is present in the biological or environmental sample.

11. The method of claim 10, wherein the method further comprises preparing a Candida lusitaniae assay sample by contacting a portion of the biological or environmental sample with magnetic particles, wherein the magnetic particles have binding moieties on their surfaces, the binding moieties operative to alter the specific aggregation of the magnetic particles in the presence of an analyte associated with Candida lusitaniae and determining whether Candida lusitaniae is present in the sample in accordance with steps (c)-(f) of the method.

12. The method of claim 10 or 11, wherein the method further comprises preparing a Candida haemulonii assay sample by contacting a portion of the biological or environmental sample with magnetic particles, wherein the magnetic particles have binding moieties on their surfaces, the binding moieties operative to alter the specific aggregation of the magnetic particles in the presence of an analyte associated with Candida haemulonii and determining whether Candida haemulonii is present in the sample in accordance with steps (c)-(f) of the method

13. The method of any one of claims 10-12, wherein the method further comprises preparing a Candida duobushaemulonii assay sample by contacting a portion of the biological or environmental sample with magnetic particles, wherein the magnetic particles have binding moieties on their surfaces, the binding moieties operative to alter the specific aggregation of the magnetic particles in the presence of an analyte associated with Candida duobushaemulonii and determining whether Candida duobushaemulonii is present in the sample in accordance with steps (c)-(f) of the method.

14. The method of any one of claims 10-13, wherein the method further comprises preparing a Candida pseudohaemulonii assay sample by contacting a portion of the biological or environmental sample with magnetic particles, wherein the magnetic particles have binding moieties on their surfaces, the binding moieties operative to alter the specific aggregation of the magnetic particles in the presence of an analyte associated with Candida pseudohaemulonii and determining whether Candida pseudohaemulonii is present in the sample in accordance with steps (c)-(f) of the method.

15. A method for detecting the presence of a Candida species cell in a biological or environmental sample, the method comprising:
(a) lysing the Candida species cells in a biological or environmental sample to form a lysate;
(b) amplifying a Candida species target nucleic acid in the lysate in the presence of a primer pair to form an amplified lysate comprising a Candida amplicon, wherein the primer pair comprises a forward primer comprising the oligonucleotide sequence: 5′-GGC ATG CCT GTT TGA GCG T-3′ (SEQ ID NO: 1) or 5′-GGG CAT GCC TGT TTG AGC GT-3′ (SEQ ID NO: 2) and a reverse primer comprising the oligonucleotide sequence: 5′-GCT TAT TGA TAT GCT TAA GTT CAG CGG GT-3′ (SEQ ID NO: 3);
(c) following step (b), contacting the amplified lysate with magnetic particles to form an assay sample, wherein the magnetic particles comprise binding moieties on their surface, the binding moieties operative to alter aggregation of the magnetic particles in the presence of the Candida species amplicon;
(d) providing the assay sample in a detection tube within a device, the device comprising a support defining a well for holding the detection tube comprising the assay sample, and having an RF coil configured to detect a signal produced by exposing the assay sample to a bias magnetic field created using one or more magnets and an RF pulse sequence;
(e) exposing the assay sample to a bias magnetic field and an RF pulse sequence;
(f) following step (e), measuring the signal from the assay sample; and
(g) on the basis of the result of step (f), determining whether a Candida species cell was present in the biological or environmental sample.

16. The method of claim 15, wherein the magnetic particles comprise a first population of magnetic particles conjugated to a first probe, and a second population of magnetic particles conjugated to a second probe, the first probe operative to bind to a first segment of the Candida species amplicon and the second probe operative to bind to a second segment of the Candida species amplicon, wherein the magnetic particles form aggregates in the presence of the Candida species amplicon.

17. The method of claim 16, wherein the Candida species is Candida auris, and the first probe comprises the oligonucleotide sequence: 5′-CTA CCT GAT TTG AGG CGA CAA CAA AAC-3′ (SEQ ID NO: 4), and the second probe comprises the oligonucleotide sequence: 5′-CCG CGA AGA TTG GTG AGA AGA CAT-3′ (SEQ ID NO: 5).

18. The method of claim 16, wherein the Candida species is Candida lusitaniae, and the first probe comprises the oligonucleotide sequence: 5′-CCT ACC TGA TTT GAG GGC GAA ATG TC-3′ (SEQ ID NO: 6), and the second probe comprises the oligonucleotide sequence: 5′-GGA GCA ACG CCT AAC CGG G-3′ (SEQ ID NO: 7).

19. The method of claim 16, wherein the Candida species is Candida haemulonii, and the first probe comprises the oligonucleotide sequence: 5′-GTC CTA CCT GAT TTG AGG GGA AAA AGC-3′ (SEQ ID NO: 8), and the second probe comprises the oligonucleotide sequence: 5′-AAC AAA TCC ACC AAC GGT GAG AAG ATA T-3′ (SEQ ID NO: 9).

20. The method of claim 16, wherein the Candida species is Candida duobushaemulonii, and the first probe comprises the oligonucleotide sequence: 5′-CGT AGA CTT CGC TGC GGA T-3′ (SEQ ID NO: 48) or 5′-GCG TAG ACT TCG CTG CGG AT-3′ (SEQ ID NO: 28), and the second probe comprises the oligonucleotide sequence: 5′-CTG GGC GGT GAG AAG AAA TC-3′ (SEQ ID NO: 29).

21. The method of claim 16, wherein the Candida species is Candida pseudohaemulonii, and the first probe comprises the oligonucleotide sequence: 5′-GCG TAG ACT TCG CTG CTG GAA-3′ (SEQ ID NO: 30), and the second probe comprises the oligonucleotide sequence: 5′-CCG TGC GGT GAG AAG AAA TC-3′ (SEQ ID NO: 31).

22. The method of claim 16, wherein the Candida species is Candida duobushaemulonii or Candida pseudohaemulonii, and the first probe comprises the oligonucleotide sequence: 5′-TCC TAC CTG ATT TGA GGA AAT AGC ATG G-3′ (SEQ ID NO: 32), and the second probe comprises the oligonucleotide sequence: 5′-ATT TAG CGG ATG CAA AAC CAC C-3′ (SEQ ID NO: 33).

23. The method of any one of claims 1-22, wherein the biological or environmental sample or portion thereof is between 0.1 and 4 mL.

24. The method of claim 23, wherein the biological or environmental sample is between 1.25 and 2 mL.

25. The method of any one of claims 1-24, wherein the biological or environmental sample is blood, a swab, cerebrospinal fluid (CSF), urine, or synovial fluid.

26. The method of claim 25, wherein the biological sample is blood.

27. The method of claim 26, wherein the blood is whole blood.

28. The method of claim 26 or 27, wherein amplifying is in the presence of whole blood proteins and non-target nucleic acids.

29. The method of any one of claims 15-28, wherein lysing comprises mechanical lysis or heat lysis.

30. The method of claim 29, wherein the mechanical lysis is beadbeating or sonicating.

31. The method of any one of claims 1-30, wherein the steps of the method are completed within 5 hours.

32. The method of claim 31, wherein the steps of the method are completed within 4 hours.

33. The method of claim 32, wherein the steps of the method are completed within 3 hours.

34. The method of any one of claims 10-33, wherein the assay sample is contacted with 1×10⁶ to 1×10¹³ magnetic particles per milliliter of the biological or environmental sample.

35. The method of any one of claims 10-34, wherein measuring the signal of the assay sample comprises measuring the T₂ relaxation response of the assay sample, and wherein increasing agglomeration in the assay sample produces an increase in the observed T₂ relaxation time of the assay sample.

36. The method of any one of claims 9-35, wherein the magnetic particles have a mean diameter of from 150 nm to 699 nm or from 700 nm to 1200 nm.

37. The method of claim 36, wherein the magnetic particles have a mean diameter of from 700 nm to 950 nm.

38. The method of claim 37, wherein the magnetic particles have a mean diameter of from 700 nm to 850 nm.

39. The method of any one of claims 9-38, wherein the magnetic particles have a T₂ relaxivity per particle of from 1×10⁹ to 1×10¹² mM⁻¹s⁻¹.

40. The method of any one of claims 9-39, wherein the magnetic particles are substantially monodisperse.

41. A method for detecting the presence of a Candida species in a whole blood sample, wherein the Candida species is Candida auris, the method comprising:
(a) providing an extract produced by lysing the red blood cells in a whole blood sample from a subject, centrifuging the sample to form a supernatant and a pellet, discarding some or all of the supernatant, and resuspending the pellet to form an extract, optionally washing the pellet prior to resuspending the pellet and optionally repeating the centrifuging, discarding, and resuspending steps;
(b) lysing cells in the extract to form a lysate;
(c) amplifying a Candida species target nucleic acid in the lysate to form an amplified lysate solution;
(d) following step (c), adding to the amplified lysate solution from 1×10⁶ to 1×10¹³ magnetic particles per milliliter of the amplified lysate solution to form a mixture, wherein the magnetic particles have a mean diameter of from 700 nm to 950 nm and binding moieties on their surface, the binding moieties operative to alter aggregation of the magnetic particles in the presence of a target nucleic acid, wherein said magnetic particles have a T₂ relaxivity per particle of from 1×10⁹ to 1×10¹² mM⁻¹s⁻¹;
(e) providing the mixture in a detection tube within a device, the device comprising a support defining a well for holding the detection tube comprising the magnetic particles and the target nucleic acid, and having an RF coil disposed about the well, the RF coil configured to detect a signal produced by exposing the mixture to a bias magnetic field created using one or more magnets and an RF pulse sequence;
(f) exposing the mixture to a bias magnetic field and an RF pulse sequence;
(g) following step (f), measuring the signal from the detection tube;
(h) on the basis of the result of step (g), detecting the target nucleic acid, wherein step (g) is carried out without any prior purification of the amplified lysate solution; and
(i) on the basis of the result of step (h), determining whether the Candida species was present in the sample.

42. The method of claim 41, wherein said whole blood sample is from 0.05 to 4.0 mL.

43. The method of claim 41 or 42, wherein step (g) comprises measuring the T₂ relaxation response of the mixture, and wherein increasing agglomeration in the mixture produces an increase in the observed T₂ relaxation time of the mixture.

44. The method of any one of claims 41-43, wherein the amplifying of step (c) comprises amplifying a nucleic acid to be detected in the presence of a forward primer and a reverse primer, each of which is universal to multiple Candida species to form a solution comprising a Candida amplicon; and said magnetic particles of step (d) have a first probe and a second probe conjugated to their surface, the first probe operative to bind to a first segment of the target nucleic acid and the second probe operative to bind to a second segment of the target nucleic acid, wherein the magnetic particles form aggregates in the presence of the target nucleic acid.

45. The method of claim 44, wherein the forward primer comprises the oligonucleotide sequence: 5′-GGC ATG CCT GTT TGA GCG T-3′ (SEQ ID NO: 1) or 5′-GGG CAT GCC TGT TTG AGC GT-3′ (SEQ ID NO: 2).

46. The method of claim 45, wherein the forward primer comprises the oligonucleotide sequence: 5′-GGC ATG CCT GTT TGA GCG T-3′ (SEQ ID NO: 1).

47. The method of any one of claims 44-46, wherein the reverse primer comprises the oligonucleotide sequence: 5′-GCT TAT TGA TAT GCT TAA GTT CAG CGG GT-3′ (SEQ ID NO: 3).

48. The method of any one of claims 44-47, wherein the first probe comprises the oligonucleotide sequence: 5′-CTA CCT GAT TTG AGG CGA CAA CAA AAC-3′ (SEQ ID NO: 4), and the second probe comprises the oligonucleotide sequence: 5′-CCG CGA AGA TTG GTG AGA AGA CAT-3′ (SEQ ID NO: 5).

49. The method of any one of claims 44-48, wherein the magnetic particles comprise two populations, a first population bearing the first probe on its surface, and the second population bearing the second probe on its surface.

50. The method of any one of claims 44-49, wherein the method further comprises determining whether Candida lusitaniae is present in the sample.

51. The method of any one of claims 44-50, wherein the method further comprises determining whether Candida haemulonii is present in the sample.

52. The method of any one of claims 44-51, wherein the method further comprises determining whether Candida duobushaemulonii is present in the sample.

53. The method of any one of claims 44-52, wherein the method further comprises determining whether Candida pseudohaemulonii is present in the sample.

54. The method of any one of claims 10-53, wherein the method detects a concentration of Candida species of 10 cells/mL of biological or environmental sample or less.

55. A composition comprising:
(a) a liquid sample, wherein the liquid sample
(i) is suspected of containing at least one Candida auris target nucleic acid, or
(ii) contains at least one Candida auris target nucleic acid amplicon generated from an amplification reaction; and
(b) within the liquid sample, from 1×10⁶ to 1×10¹³ magnetic particles per milliliter of the liquid sample, the magnetic particles having a mean diameter of from 700 nm to 950 nm, a T₂ relaxivity per particle of from 1×10⁴ to 1×10¹² mM⁻¹s⁻¹, the magnetic particles comprising a first population and a second population, the first population having a first nucleic acid probe conjugated to their surface and the second population having a second nucleic acid probe conjugated to their surface, wherein the first probe comprises the oligonucleotide sequence: 5′-CTA CCT GAT TTG AGG CGA CAA CAA AAC-3′ (SEQ ID NO: 4), and the second probe comprises the oligonucleotide sequence: 5′-CCG CGA AGA TTG GTG AGA AGA CAT-3′ (SEQ ID NO: 5).

56. A composition comprising:
(a) a liquid sample, wherein the liquid sample
(i) is suspected of containing at least one Candida lusitaniae target nucleic acid, or
(ii) contains at least one Candida lusitaniae target nucleic acid amplicon generated from an amplification reaction; and
(b) within the liquid sample, from 1×10⁶ to 1×10¹³ magnetic particles per milliliter of the liquid sample, the magnetic particles having a mean diameter of from 700 nm to 950 nm, a T₂ relaxivity per particle of from 1×10⁴ to 1×10¹² mM⁻¹s⁻¹, the magnetic particles comprising a first population and a second population, the first population having a first nucleic acid probe conjugated to their surface and the second population having a second nucleic acid probe conjugated to their surface, wherein the first probe comprises the oligonucleotide sequence: 5′-CCT ACC TGA TTT GAG GGC GAA ATG TC-3′ (SEQ ID NO: 6), and the second probe comprises the oligonucleotide sequence: 5′-GGA GCA ACG CCT AAC CGG G-3′ (SEQ ID NO: 7).

57. A composition comprising:
(a) a liquid sample, wherein the liquid sample
(i) is suspected of containing at least one Candida haemulonii target nucleic acid, or
(ii) contains at least one Candida haemulonii target nucleic acid amplicon generated from an amplification reaction; and
(b) within the liquid sample, from 1×10⁶ to 1×10¹³ magnetic particles per milliliter of the liquid sample, the magnetic particles having a mean diameter of from 700 nm to 950 nm, a T₂ relaxivity per particle of from 1×10⁴ to 1×10¹² mM⁻¹s⁻¹, the magnetic particles comprising a first population and a second population, the first population having a first nucleic acid probe conjugated to their surface and the second population having a second nucleic acid probe conjugated to their surface, wherein the first probe comprises the oligonucleotide sequence: 5′-GTC CTA CCT GAT TTG AGG GGA AAA AGC-3′ (SEQ ID NO: 8), and the second probe comprises the oligonucleotide sequence: 5′-AAC AAA TCC ACC AAC GGT GAG AAG ATA T-3′ (SEQ ID NO: 9).

58. A composition comprising:
(a) a liquid sample, wherein the liquid sample
(i) is suspected of containing at least one Candida duobushaemulonii target nucleic acid, or
(ii) contains at least one Candida duobushaemulonii target nucleic acid amplicon generated from an amplification reaction; and
(b) within the liquid sample, from 1×10⁶ to 1×10¹³ magnetic particles per milliliter of the liquid sample, the magnetic particles having a mean diameter of from 700 nm to 950 nm, a T₂ relaxivity per particle of from 1×10⁴ to 1×10¹² mM⁻¹s⁻¹, the magnetic particles comprising a first population and a second population, the first population having a first nucleic acid probe conjugated to their surface and the second population having a second nucleic acid probe conjugated to their surface, wherein the first probe comprises the oligonucleotide sequence: 5′-CGT AGA CTT CGC TGC GGA T-3′ (SEQ ID NO: 48) or 5′-GCG TAG ACT TCG CTG CGG AT-3′ (SEQ ID NO: 28), and the second probe comprises the oligonucleotide sequence: 5′-CTG GGC GGT GAG AAG AAA TC-3′ (SEQ ID NO: 29).

59. A composition comprising:
(a) a liquid sample, wherein the liquid sample
(i) is suspected of containing at least one Candida pseudohaemulonii target nucleic acid, or
(ii) contains at least one Candida pseudohaemulonii target nucleic acid amplicon generated from an amplification reaction; and
(b) within the liquid sample, from 1×10⁶ to 1×10¹³ magnetic particles per milliliter of the liquid sample, the magnetic particles having a mean diameter of from 700 nm to 950 nm, a T₂ relaxivity per particle of from 1×10⁴ to 1×10¹² mM⁻¹s⁻¹, the magnetic particles comprising a first population and a second population, the first population having a first nucleic acid probe conjugated to their surface and the second population having a second nucleic acid probe conjugated to their surface, wherein the first probe comprises the oligonucleotide sequence: 5′-GCG TAG ACT TCG CTG CTG GAA-3′ (SEQ ID NO: 30), and the second probe comprises the oligonucleotide sequence: 5′-CCG TGC GGT GAG AAG AAA TC-3′ (SEQ ID NO: 31).

60. A composition comprising:
(a) a liquid sample, wherein the liquid sample
(i) is suspected of containing at least one Candida duobushaemulonii or Candida pseudohaemulonii target nucleic acid, or
(ii) contains at least one Candida duobushaemulonii or Candida pseudohaemulonii target nucleic acid amplicon generated from an amplification reaction; and
(b) within the liquid sample, from 1×10⁶ to 1×10¹³ magnetic particles per milliliter of the liquid sample, the magnetic particles having a mean diameter of from 700 nm to 950 nm, a T₂ relaxivity per particle of from 1×10⁴ to 1×10¹² mM⁻¹s⁻¹, the magnetic particles comprising a first population and a second population, the first population having a first nucleic acid probe conjugated to their surface and the second population having a second nucleic acid probe conjugated to their surface, wherein the first probe comprises the oligonucleotide sequence: 5′-TCC TAC CTG ATT TGA GGA AAT AGC ATG G-3′ (SEQ ID NO: 32), and the second probe comprises the oligonucleotide sequence: 5′-ATT TAG CGG ATG CAA AAC CAC C-3′ (SEQ ID NO: 33).

61. A removable cartridge comprising a plurality of wells, wherein the removable cartridge comprises one or more of the following:
(a) a first well comprising the composition of claim 55;
(b) a second well comprising the composition of claim 56; and
(c) a third well comprising the composition of claim 57.

62. The removable cartridge of claim 61, wherein the removable cartridge comprises (a) through (c).

63. The removable cartridge of claim 61 or 62, further comprising
(d) a fourth well comprising the composition of claim 58;
(e) a fifth well comprising the composition of claim 59; and/or
(f) a sixth well comprising the composition of claim 60.

64. The removable cartridge of any one of claims 61-63, further comprising one or more chambers for holding a plurality of reagent modules for holding one or more assay reagents.

65. The removable cartridge of any one of claims 61-64, further comprising a chamber comprising beads for lysing cells.

66. The removable cartridge of any one of claims 61-65, further comprising a chamber comprising a polymerase.

67. The removable cartridge of any one of claims 61-66, further comprising a chamber comprising one or more primers.

68. The removable cartridge of claim 67, wherein the one or more primers comprise oligonucleotide sequences selected from SEQ ID NOs: 1, 2, and 3.

69. A method for diagnosing a disease in a subject, the method comprising:
(a) providing a biological sample obtained from the subject; and
(b) detecting the presence of a Candida species in the biological sample according to the method of any one of claims 1-54,
wherein the presence of a Candida species in the biological sample obtained from the subject identifies the subject as one who may have the disease.

70. A method for infection control, decolonization, or epidemiological monitoring, the method comprising:
(a) providing an environmental sample; and
(b) detecting the presence of a Candida species in the environmental sample according to the method of any one of claims 1-40,
wherein the presence of a Candida species in the environmental sample is used for infection control, decolonization, or epidemiological monitoring.

71. The method of claim 69 or 70, wherein the Candida species is Candida auris.

72. The method of claim 69 or 70, wherein the Candida species is Candida lusitaniae.

73. The method of claim 69 or 70, wherein the Candida species is Candida haemulonii.

74. The method of claim 69 or 70, wherein the Candida species is Candida duobushaemulonii.

75. The method of claim 69 or 70, wherein the Candida species is Candida pseudohaemulonii.

76. A method for treating a disease in a subject, the method comprising administering an effective amount of a therapeutic agent to the subject, wherein the subject has been diagnosed as having the disease based on detecting the presence of a Candida species according to the method of any one of claims 69 or 71-75.

77. The method of claim 76, wherein the Candida species is Candida auris, Candida lusitaniae, Candida haemulonii, Candida duobushaemulonii, or Candida pseudohaemulonii.

78. The method of any one of claims 69 or 71-77, wherein the disease is Candidiasis, Candidemia, or sepsis.

79. The method of any one of claims 76-78, wherein the therapeutic agent is an antifungal agent.

80. The method of claim 79, wherein the subject has a Candida auris infection and the antifungal agent is a 1,3-β-D-glucan synthesis inhibitor.

81. The method of claim 80, wherein the 1,3-β-D-glucan synthesis inhibitor is caspofungin, anidulafungin, micafungin, enfumafungin, or SCY-078.


SEQUENCE LISTING (56 sequences)

SEQ ID NO: 1 - DNA, 19 bp, Artificial Sequence
  Synthetic Construct
ggcatgcctgtttgagcgt

SEQ ID NO: 2 - DNA, 20 bp, Artificial Sequence
  Synthetic Construct
gggcatgcctgtttgagcgt

SEQ ID NO: 3 - DNA, 29 bp, Artificial Sequence
  Synthetic Construct
gcttattgatatgcttaagttcagcgggt

SEQ ID NO: 4 - DNA, 27 bp, Artificial Sequence
  Synthetic Construct
ctacctgatttgaggcgacaacaaaac

SEQ ID NO: 5 - DNA, 24 bp, Artificial Sequence
  Synthetic Construct
ccgcgaagattggtgagaagacat

SEQ ID NO: 6 - DNA, 26 bp, Artificial Sequence
  Synthetic Construct
cctacctgatttgagggcgaaatgtc

SEQ ID NO: 7 - DNA, 19 bp, Artificial Sequence
  Synthetic Construct
ggagcaacgcctaaccggg

SEQ ID NO: 8 - DNA, 27 bp, Artificial Sequence
  Synthetic Construct
gtcctacctgatttgaggggaaaaagc

SEQ ID NO: 9 - DNA, 28 bp, Artificial Sequence
  Synthetic Construct
aacaaatccaccaacggtgagaagatat

SEQ ID NO: 10 - DNA, 20 bp, Artificial Sequence
  Synthetic Construct
ggcatgcctgtttgagcgtc

SEQ ID NO: 11 - DNA, 23 bp, Artificial Sequence
  Synthetic Construct
acccagcggtttgagggagaaac

SEQ ID NO: 12 - DNA, 30 bp, Artificial Sequence
  Synthetic Construct
aaagtttgaagatatacgtggtggacgtta

SEQ ID NO: 13 - DNA, 21 bp, Artificial Sequence
  Synthetic Construct
cgcacgcgcaagatggaaacg

SEQ ID NO: 14 - DNA, 23 bp, Artificial Sequence
  Synthetic Construct
aagttcagcgggtattcctacct

SEQ ID NO: 15 - DNA, 27 bp, Artificial Sequence
  Synthetic Construct
agctttttgttgtctcgcaacactcgc

SEQ ID NO: 16 - DNA, 26 bp, Artificial Sequence
  Synthetic Construct
ctaccaaacacaatgtgtttgagaag

SEQ ID NO: 17 - DNA, 31 bp, Artificial Sequence
  Synthetic Construct
cctgatttgaggtcaaacttaaagacgtctg

SEQ ID NO: 18 - DNA, 24 bp, Artificial Sequence
  Synthetic Construct
  misc_feature (22)..(22): n is 5′ 5-nitroindole
agtcctacctgatttgaggtcnaa

SEQ ID NO: 19 - DNA, 21 bp, Artificial Sequence
  Synthetic Construct
  misc_feature (4)..(4): n is 5′ 5-nitroindole
ccgngggtttgagggagaaat

SEQ ID NO: 20 - DNA, 33 bp, Artificial Sequence
  Synthetic Construct
aaagttatgaaataaattgtggtggccactagc

SEQ ID NO: 21 - DNA, 22 bp, Artificial Sequence
  Synthetic Construct
acccgggggtttgagggagaaa

SEQ ID NO: 22 - DNA, 24 bp, Artificial Sequence
  Synthetic Construct
agtcctacctgatttgaggtcgaa

SEQ ID NO: 23 - DNA, 21 bp, Artificial Sequence
  Synthetic Construct
ccgagggtttgagggagaaat

SEQ ID NO: 24 - DNA, 200 bp, Artificial Sequence
  Synthetic Construct
ggcatgcctgtttgagcgtcctgcatcatactgaaatagatccttcgacaacctcggtacactgggaacaaggcctcaaacattgatgctcgactacacgtagggcaatgcgtcttgctagaagcgaaatctgtggcttgctagtgcaagctggtcggcgtattattccaacccgctgaacttaagcatatcaataagca

SEQ ID NO: 25 - DNA, 200 bp, Artificial Sequence
  Synthetic Construct
gcttattgatatgcttaagttcagcgggttggaataatacgccgaccagcttgcactagcaagccacagatttcgcttctagcaagacgcattgccctacgtgtagtcgagcatcaatgtttgaggccttgttcccagtgtaccgaggttgtcgaaggatctatttcagtatgatgcaggacgctcaaacaggcatgcca

SEQ ID NO: 26 - DNA, 33 bp, Artificial Sequence
  Synthetic Construct
ggttgtcgaaggatctatttcagtatgatgcag

SEQ ID NO: 27 - DNA, 29 bp, Artificial Sequence
  Synthetic Construct
tggaataatacgccgaccagcttgcacta

SEQ ID NO: 28 - DNA, 20 bp, Artificial Sequence
  Synthetic Construct
gcgtagacttcgctgcggat

SEQ ID NO: 29 - DNA, 20 bp, Artificial Sequence
  Synthetic Construct
ctgggcggtgagaagaaatc

SEQ ID NO: 30 - DNA, 21 bp, Artificial Sequence
  Synthetic Construct
gcgtagacttcgctgctggaa

SEQ ID NO: 31 - DNA, 20 bp, Artificial Sequence
  Synthetic Construct
ccgtgcggtgagaagaaatc

SEQ ID NO: 32 - DNA, 28 bp, Artificial Sequence
  Synthetic Construct
tcctacctgatttgaggaaatagcatgg

SEQ ID NO: 33 - DNA, 22 bp, Artificial Sequence
  Synthetic Construct
atttagcggatgcaaaaccacc

SEQ ID NO: 34 - DNA, 249 bp, Artificial Sequence
  Synthetic Construct
ggaaatctaacgagagagcatgctcctgcggccccggagacggtgcgccgcggggtgcggcgccttctttcacatgtatccaaaacgtctctcggcaacggatatctcggctctcgcatcgatgaagaacgtagcgaaatgcgatacttggtgtgaattgcagaatcccgtgaaccatcgagtctttgaacgcaagttgcgccccaagccattaggccgagggcacgtctgcctgggtgtcacgcatcg

SEQ ID NO: 35 - DNA, 24 bp, Artificial Sequence
  Synthetic Construct
ggaaatctaacgagagagcatgct

SEQ ID NO: 36 - DNA, 23 bp, Artificial Sequence
  Synthetic Construct
ggaaatctaacgagagagcatgc

SEQ ID NO: 37 - DNA, 19 bp, Artificial Sequence
  Synthetic Construct
cgatgcgtgacacccaggc

SEQ ID NO: 38 - DNA, 18 bp, Artificial Sequence
  Synthetic Construct
gatgcgtgacacccaggc

SEQ ID NO: 39 - DNA, 30 bp, Artificial Sequence
  Synthetic Construct
gagacgttttggatacatgtgaaagaaggc

SEQ ID NO: 40 - DNA, 26 bp, Artificial Sequence
  Synthetic Construct
cgatggttcacgggattctgcaattc

SEQ ID NO: 41 - DNA, 191 bp, Artificial Sequence
  Synthetic Construct
gatgcagcaacaacagattccttgcttctcatacaataacatgacaaaccccattaataaaaacgcggtccacttatcatacagaatatcagatagtggcaattaattgtgacaaaaattcgaaagttgtgtacagttcttcattgttcgaaaaattgttatgacaagatacaccaggacataacggctac

SEQ ID NO: 42 - DNA, 18 bp, Artificial Sequence
  Synthetic Construct
gcagcaacaacagattcc

SEQ ID NO: 43 - DNA, 20 bp, Artificial Sequence
  Synthetic Construct
gtagccgttatgtcctggtg

SEQ ID NO: 44 - DNA, 31 bp, Artificial Sequence
  Synthetic Construct
tcgaacaatgaagaactgtacacaactttcg

SEQ ID NO: 45 - DNA, 30 bp, Artificial Sequence
  Synthetic Construct
ggtttgtcatgttattgtatgagaagcaag

SEQ ID NO: 46 - DNA, 20 bp, Artificial Sequence
  Synthetic Construct
  misc_feature (20)..(20): n is 5′ 5-nitroindole
ggcatgcctgtttgagcgtn

SEQ ID NO: 47 - DNA, 20 bp, Artificial Sequence
  Synthetic Construct
  misc_feature (20)..(20): n is deoxyinosine
ggcatgcctgtttgagcgtn

SEQ ID NO: 48 - DNA, 19 bp, Artificial Sequence
  Synthetic Construct
cgtagacttcgctgcggat

SEQ ID NO: 49 - DNA, 36 bp, Artificial Sequence
  Synthetic Construct
tttttttttctacctgatttgaggcgacaacaaaac

SEQ ID NO: 50 - DNA, 33 bp, Artificial Sequence
  Synthetic Construct
ccgcgaagattggtgagaagacatttttttttt

SEQ ID NO: 51 - DNA, 28 bp, Artificial Sequence
  Synthetic Construct
tttttttttcgtagacttcgctgcggat

SEQ ID NO: 52 - DNA, 29 bp, Artificial Sequence
  Synthetic Construct
ctgggcggtgagaagaaatcttttttttt

SEQ ID NO: 53 - DNA, 36 bp, Artificial Sequence
  Synthetic Construct
tttttttttgtcctacctgatttgaggggaaaaagc

SEQ ID NO: 54 - DNA, 37 bp, Artificial Sequence
  Synthetic Construct
aacaaatccaccaacggtgagaagatatttttttttt

SEQ ID NO: 55 - DNA, 35 bp, Artificial Sequence
  Synthetic Construct
tttttttttcgatggttcacgggattctgcaattc

SEQ ID NO: 56 - DNA, 39 bp, Artificial Sequence
  Synthetic Construct
gagacgttttggatacatgtgaaagaaggcttttttttt